(19) United States
(12) Patent Application Publication
(10) Pub. No.: US 2017/0209579 A1
(43) Pub. Date: Jul. 27, 2017

(54) NON-INVASIVE RADIOFREQUENCY FIELD TREATMENT FOR CANCER THERAPY

(71) Applicants: Baylor College of Medicine, Houston, TX (US); Board of Regents, The University of Texas System, Austin, TX (US); The Methodist Hospital, Houston, TX (US)

(72) Inventors: Steven Curley, Missouri City, TX (US); Stuart Corr, Houston, TX (US); Rita Serda, Pearland, TX (US); Mustafa Raoof, Monrovia, CA (US)

(21) Appl. No.: 15/328,418

(22) PCT Filed: Jul. 24, 2015
(86) PCT No.: PCT/US15/42118

§ 371 Date: Jan. 23, 2017

Related U.S. Application Data
(60) Provisional application No. 62/028,604, filed on Jul. 24, 2014.

(51) Int. Cl. A61K 41/00; A61K 31/337; A61B 18/18; A61B 5/00; A61N 1/40; A61B 5/01; A61B 5/055; A61K 31/7068; A61M 37/00

(52) U.S. Cl. CPC A61K 41/0052; A61K 31/7068; A61K 31/337; A61K 47/48284; A61B 18/18; A61M 37/00; A61N 1/406; A61B 5/015; A61B 5/055; A61B 5/4839; A61B 5/4848; A61B 2018/1807; A61M 2037/0007

(57) ABSTRACT

Embodiments of the disclosure concern methods and compositions for treating a subject with cancer, including overcoming resistance to a cancer drug using non-invasive radiotherapy frequency in combination with cancer drugs. In particular embodiments, radiofrequency therapy and cancer drugs in combination provide a synergistic benefit for cancer therapy. Use of radiofrequency therapy is an effective means to facilitate transport and perfusion of the cancer drugs in the subject.

This application claims priority to U.S. Provisional Patent Application Ser. No. 62/028,604, filed Jul. 24, 2014, which is incorporated by reference herein in its entirety.

### STATEMENT REGARDING FEDERALLY SPONSORED RESEARCH OR DEVELOPMENT

This invention was made with government support under Grant Number U54CA143837 awarded by the National Cancer Institute. The government has certain rights in the invention.

### TECHNICAL FIELD

Embodiments of the disclosure include at least the fields of cell biology, molecular biology, radiofrequency therapies, hyperthermia, and medicine, including cancer medicine.

### BACKGROUND

The interactions of high-frequency radiowaves (13.56 MHz) with biological tissues and nanoparticles are currently being investigated as a new platform for non-invasive cancer therapy. The differential dielectric properties of cancerous and normal tissues cause radiofrequency (RF) energy to be selectively absorbed and converted to heat to a greater extent in tumors. This heating is due to larger dielectric losses within tumor tissue compared to normal tissues (Raoof, et al., 2013). Also, due to long RF wavelengths (22 m), tissue penetration depths up to 30 cm are achievable.

In a bid to increase differential-heating rates even further, several studies have shown the heating characteristics (Corr, et al., 2012; Li, et al., 2011; Kruse, et al., 2011); induced biological toxicity (Koshkina, et al., 2014; Con, et al., 2013; Raoof, et al., 2012a; Raoof, et al., 2012b; Glazer, et al., 2010a; Glazer, et al., 2010b; Gannon, et al., 2008; Gannon, et al., 2007); electrical interactions (Dongxiao, et al., 2012; Hanson, et al., 2011; Hanson, et al., 2009); and feasibility (Kim, et al., 2013) of nanomaterial interactions with radiofrequency energy and their use as a potential medical hyperthermia adjuvant. Gold nanoparticles; carbon nanotubes; quantum dots; and C60 fullerenes have all received great attention. Gold is of special significance as it is already medically approved and has low-level toxicity in mammals. Previous work has shown that gold nanoparticles conjugated to cancer-targeting antibodies, upon radiofrequency exposure to cells or tissue enhances hyperthermia. The deposited thermal dose into the tumor is enhanced from the small, yet-significant, radiofrequency absorption cross-section. The non-invasive ‘targeted-radiofrequency-hyperthermia’ approach has not only been employed for localized hyperthermia but has also been used to ‘trigger-and-release’ therapeutic payloads encapsulated within carbon nanomaterials (Raoof, et al., 2013). A recent study looked at the efficacy of cisplatin-loaded ultrashort (20-40 nm) carbon nanotubes, wrapped in a pluronic surfactant to minimize passive cisplatin release. Under radiofrequency exposure the pluronic underwent a gradual phase transition, unwrapping itself from the nanotube, thereby releasing the therapeutic cargo.

A means of visualizing the dynamics; interplay; and distribution of chemotherapy agents in vivo under non-invasive radiofrequency conditions would give more insight into the fundamental processes and basic science behind this therapy. However, as of yet there has been no design for capturing these dynamic events. The present disclosure provides embodiments of such means and other therapeutic aspects, satisfying a long-felt need in the art.

### BRIEF SUMMARY

Embodiments of the disclosure include methods and compositions for the treatment and/or prevention of cancer in a mammal, including at least cancer that is resistant to one or more therapies. In specific embodiments, the therapy is one or more cancer drugs. The one or more cancer drugs may be of any kind. In one embodiment, the therapy is a chemotherapy, a hormone, an immunomodulatory agent, a cell-based carrier, or a combination thereof. Embodiments contemplate the use of radiofrequency energy as a cancer therapy and, in particular embodiments, non-invasive radiofrequency electric field exposure is used for treatment in an individual with cancer in conjunction with at least one or more drugs to which it is known an individual can develop or has developed resistance or to which it is suspected that an individual can develop resistance.

In particular embodiments, synergism between the non-invasive radiofrequency therapy and the cancer drug(s) provides a benefit to the individual being treated that otherwise would not be provided if the cancer drug(s) was utilized by itself in therapy. In specific embodiments, an amount of the cancer drug(s) being utilized with the non-invasive radiofrequency therapy is less than the amount of the cancer drug(s) that would be used without the non-invasive radiofrequency therapy. In certain embodiments, the synergism between the non-invasive radiofrequency therapy and the cancer drug allows for enhanced transport and perfusion of the cancer drug compared to that which occurs when the cancer drug is utilized alone.

In specific embodiments, an individual is treated with the non-invasive radiofrequency therapy and the cancer drug and is not also treated with one or more other therapies or moieties, such as a radiofrequency absorption enhancer. In particular embodiments, the individual is treated with the non-invasive radiofrequency therapy and the cancer drug but is not treated with a therapy that comprises a metal particle, such as a metal particle alone or a metal particle that is linked to another compound. Thus, in particular aspects, the individual is not provided a composition that increases the tendency of a target area of the radiofrequency therapy to absorb more energy from the radiofrequency signal. In particular embodiments, the methods do not employ a non-invasive radiofrequency absorption enhancer, including a non-invasive radiofrequency absorption enhancer that was specifically selected to act as a non-invasive radiofrequency absorption enhancer in the methods. In particular embodiments, the course of action to refrain from using one or more radiofrequency absorption enhancers is an intentional course of action with the purpose of meaning to refrain from using the enhancer(s).

In certain embodiments, the combination of the non-invasive radiofrequency therapy and the cancer drug(s) that is known or suspected of incurring chemoresistance provides physiological benefits that would not otherwise occur if either of the two therapies were utilized alone and/or that do not occur with other combinations. In particular cases, the methods of the disclosure provide improvements in transport and uptake of certain cancer drugs, including cancer drugs that are systemically circulating in the blood of the individual, for example. In other aspects, however, the transport and uptake of certain cancer drugs are improved when the cancer drug is given to the individual in a manner that does not result in blood circulation, such as by intratumoral injection, for example.

In one embodiment of the disclosure, there is a method of increasing perfusion into particular tissues or cells of an individual of one or more cancer drugs that are delivered to the individual systemically or non-systemically, wherein the cancer drug and non-invasive radiofrequency therapy are provided to the individual at the same time or at different times. When the cancer drug and non-invasive radiofrequency therapy are provided at different times, they may be provided in any suitable order.

In some embodiments, there is a method of increasing intra-tumoral blood vessel permeability for uptake of a cancer drug in an individual, comprising the steps of providing to the individual an effective amount of both non-invasive radiofrequency therapy and the cancer drug. In specific embodiments, the increase may be measured as improvement of at least one symptom of the cancer; reduction in the size of a tumor; reduction in metastasis of the cancer; reduction in tumor load, and/or reduction in the amount of cancer drug and/or duration of its administration that is employed and yet is still effective for cancer treatment; and so forth.

Embodiments of the disclosure include interactions of high-frequency radiowaves (such as at 13.56 MHz) or medically-approved research frequencies (across the bandwidth 20 HZ-1 GHz) or 13.56 MHz radiowaves amplitude modulated at lower frequencies across the range (20 Hz-13.55 MHz) or pulsed radiowaves of pulse duration 1 ns (1×10⁻⁹ seconds)−1 s, with biological tissues for non-invasive cancer therapy for an individual that is being treated with one or more drugs known for resistance development or suspected of incurring resistance, or with drugs or particles known to cause mechanical damage and thereby sensitize the cancer cells to radiofrequency therapy.

In specific embodiments, the radiofrequency therapy system utilized in methods of the disclosure has the capability of imaging of a labeled compound used for the therapy and/or for imaging biological changes that occur as a result of use of the non-invasive radiofrequency therapy. In specific embodiments, confocal microscopy, intravital microscopy, and/or multiphoton microscopy is used as the imaging component of the non-invasive radiofrequency therapy system. In certain embodiments, the imaging method with the non-invasive radiofrequency allows for real-time imaging and quantitation of vascular permeability, tissue integrity, nanoparticle accumulation, tissue penetration, tissue necrosis, tissue apoptosis, tissue DNA replication phase, blood flow dynamics, and cellular migration events, all as a function of non-invasive radiofrequency electric-field exposure. In specific embodiments the non-invasive radiofrequency therapy system is portable, although it may not be portable in some circumstances. In certain aspects, there is an imaging system for the non-invasive radiofrequency therapy that has a portable radiofrequency generator to allow for in vivo fluorescent imaging of cancer under radiofrequency exposure for non-invasive radiofrequency cancer therapy. In specific embodiments, the use of the non-invasive radiofrequency therapy system allows real-time monitoring of non-invasive radiofrequency-induced changes in tumor environment or microenvironment.

In particular embodiments, there is provided non-invasive radiofrequency field treatment to enhance tumor blood flow and delivery of one or more compositions, including at least drugs, biologic agents, and/or nanomaterials, to cancer cells, including at least to localized and/or malignant solid tumors.

In specific embodiments, the methods of the disclosure are utilized for cancers that are not chemoresistant.

In certain embodiments, the methods of the disclosure comprise, consist of, or consist essentially of the step of providing to an individual in need thereof an effective amount of non-invasive radiofrequency therapy and an effective amount of one or more cancer drugs.

In particular embodiments, non-invasive radiofrequency therapy and one or more cancer drugs are provided to an individual, such as for a method comprising the step of providing to the individual an effective amount of non-invasive radiofrequency therapy under sufficient conditions to the individual before, during, and/or after delivery of an effective amount of the cancer drug to the individual. In a specific embodiment, the amount of the cancer drug delivered to the individual is less than the amount given to the individual in the absence of providing the non-invasive radiofrequency therapy to the individual. The cancer drug may be an alkylating agent, antimetabolite, anthracycline, topoisomerase inhibitor, hormone therapy, mitotic inhibitor, proteasome inhibitor, immunotherapy, or a combination thereof.

In particular embodiments, the non-invasive radiofrequency therapy and cancer drug(s) are provided to the individual once a day or more than once a day. In certain embodiments, the non-invasive radiofrequency therapy and cancer drug(s) are provided to the individual once a week or more than once a week. In specific embodiments, the non-invasive radiofrequency therapy and cancer drug(s) are provided to the individual over the course of days, weeks or months, such as over the course of 1-7, 1-6, 1-5, 1-4, 1-3, 1-2, 2-7, 2-6, 2-5, 2-4, 2-3, 3-7, 3-6, 3-5, 3-4, 4-7, 4-6, 4-5, 5-7, 5-6, or 6-7 days or 1-4, 1-3, 1-2, 2-4, 2-3, or 3-4 weeks, or over the course of 1-12, 1-11, 1-10, 1-9, 1-8, 1-7, 1-6, 1-5, 1-4, 1-3, 1-2, 2-12, 2-11, 2-10, 2-9, 2-8, 2-7, 2-6, 2-5, 2-4, 2-3, 3-12, 3-11, 3-10, 3-9, 3-8, 3-7, 3-6, 3-5, 3-4, 4-12, 4-11, 4-10, 4-9, 4-8, 4-7, 4-6, 4-5, 5-12, 5-11, 5-10, 5-9, 5-8, 5-7, 5-6, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 months.

In specific embodiments of methods of the disclosure, for certain radiofrequency system power parameters, the non-invasive radiofrequency therapy may operate at a power between 1-1500 watts (W), such as 100-1200 W, 100-900 W, 100-500 W, 100-300 W, and 100-200 W, for example. The non-invasive radiofrequency therapy may be generated by a portable system or a stationary system. In particular aspects, the duration of exposure of the non-invasive radiofrequency therapy for the individual is between 1-60, 1-50, 1-40, 1-30, 1-20, 1-10, 1-5, 1-4, 5-10, 5-9, 5-8, 5-7, 5-6, 6-10, 6-10, 6-8, 6-7, 7-10, 7-9, 7-8, 8-10, 8-9, or 9-10 minutes. The temperature that is generated at a desired location to which the radiofrequency is directed may be between 37° C. and 45° C., for example, and other ranges include 37° C.-44° C., 37-43° C., 37° C.-42° C., 37° C.-41° C., 37° C.-40° C., 37° C.-39° C., 37° C.-38° C., 38° C.-45° C., 38° C.-44° C., 38° C.-43° C., 38° C.-42° C., 38° C.-41° C., 38° C.-40° C., 38° C.-39° C., 39° C.-45° C., 39° C.-44° C., 39° C.-43° C., 39° C.-42° C., 39° C.-41° C., 39° C.-40° C., 40° C.-45° C., 40° C.-44° C., 40° C.-43° C., 40° C.-42° C., 40° C.-41° C., 41° C.-45° C., 41° C.-44° C., 41° C.-43° C., 41° C.-42° C., 42° C.-45° C., 42° C.-44° C., 42° C.-43° C., 43° C.-45° C., 43° C.-44° C., or 44° C.-45° C. The temperature that is generated at a desired location to which the radiofrequency is directed may be 37° C., 38° C., 39° C., 40° C., 41° C., 42° C., 43° C., 44° C., or 45° C., and the temperature may be changed within a certain treatment and/or from treatment to treatment. The non-invasive radiofrequency therapy system may comprise a means of real-time in vivo imaging.

In specific embodiments, an individual receiving the therapy, that has received the therapy, or that will receive the therapy is provided an additional cancer therapy, such as a cancer drug that is not the cancer drug of the inventive therapy, radiation, and/or surgery. In specific embodiments, the cancer drug is gemcitabine, abraxane, cetuximab, nitrogen mustards, such as mechlorethamine (nitrogen mustard), chlorambucil, cyclophosphamide (Cytoxan®), ifosfamide; melphalan; nitrosoureas, such as streptozocin, carmustine (BCNU), and lomustine; alkyl sulfonates, such as busulfan; triazines, such as dacarbazine (DTIC) and temozolomide (Temodar®); ethylenimines, such as thiotepa and altretamine (hexamethylmelamine); 5-fluorouracil (5-FU); 6-mercaptopurine (6-MP); Capecitabine (Xeloda®); Cladribine; Clofarabine; Cytarabine (Ara-C®); Floxuridine; Fludarabine; Gemcitabine (Gemzar®); Hydroxyurea; Methotrexate; Pemetrexed (Alimta®); Pentostatin; Thioguanine; Daunorubicin; Doxorubicin (Adriamycin®); Epirubicin; Idarubicin; topotecan; irinotecan; any one or more conventionally known cancer drugs, or a combination thereof. In certain aspects, the methods of the disclosure further comprise the step of determining that the individual has cancer. Such determination may occur by biopsy, x-ray, magnetic resonance imaging, histology, blood test, or a combination thereof, as examples.

In one embodiment, there is a method of treating cancer in an individual, comprising the step of providing to the individual a therapy that comprises, consists of, or consists essentially of an effective amount of radiofrequency therapy under sufficient conditions to the individual before, during, and/or after delivery of an effective amount of the cancer drug to the individual.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is an example of an embodiment of a non-invasive RF system for cancer therapy. The patient is placed between the transmitting (TX) and receiving (RX) heads where a 13.56 MHz high-power electric field (as an example) is generated. As the tumor heats due to absorption of RF, waves the blood vessels dilate and become more permeable, increasing intratumoral blood flow and extravasation of macromolecules or chemotherapeutics or nanoparticles from the vessels into the tumor tissue where they are internalized by cancer cells.

FIGS. 2A-2D illustrate an example of an embodiment of a Portable RF system setup and generated electric field. (2A) Portable RF system consists of the transmitting unit (TX) and receiving head (RX) that generates a high-power electric field across the specimen (e.g. mammal). The system is driven by a variable power fixed RF amplifier (0-200 W, 13.56 MHz) that is cooled during operation by a water chiller. Heat production is monitored using an infrared (IR) camera or direct insertion of fiber optical probes. (2B) Circuit representation of the portable RF system. (2C) Setup for extracting electric-field intensities. An electric-field probe (EFP) is placed at specific points along the x- and z-axis in between the TX and RX heads and measures the voltage at each point for 20 W RF-power. (2D) The electric field is derived from the voltage data and is plotted as an intensity contour plot.

FIGS. 3A-3C illustrate an example of modulation of tumor temperature using RF exposure. (3A) Thermal fiber optic probe placement. Probes #1-3 are positioned (i) under the skin but above the tumor; (ii) under the skin in between the tumor and the main body; and (iii) under the skin next to the intraperitoneal cavity. (3B) Extracted thermal probe data. The recorded temperature of the probes was modulated by turning on and off the RF system (+RF and −RF). The system was turned off once the tumor temperature (probe #1) reached 45° C., 43° C., and 41° C., respectively, and was turned on when all probes had values in the range ˜29-31° C. (3C) The IR camera simultaneously measured the surface temperature of the points where the thermal probes were located.

FIGS. 4A-4E illustrate an example of an embodiment of an intravital microscopy (IVM)-RF system. (4A) Portable RF (p-RF) generator retrofitted to IVM system (inset shows p-RF generator with dimensions). (4B) Schematic representation of IVM-RF system. (4C) Animal manipulations for IVM-RF exposure. The mouse is placed on a Teflon stage with tumor exposed. (4D) Mouse is manipulating into place so the tumor can be imaged whilst being located in close proximity to the TX head as possible. (4E) Mouse being imaged during RF exposure (RX head has been placed on opposite side of mouse to allow for RF circuit completion).

FIGS. 5A-5E show examples of system use and outcome for an embodiment of the IVM-RF system. (5A-5D) IR camera and digital photos of 4T1 breast tumor temperature during and after RF exposure (T=0.5 mins and 10 mins, respectively). (5E) Temperature versus RF exposure for two different mice. Temperature variations between two mice are due to tumor size; proximity in relation to TX head; and blood flow dynamics.

FIGS. 6A-6E illustrate an example of an embodiment of the use of RF radiowaves to disrupt and degrade tumor vessels. (6A-6D) Impact of RF exposure on vessel architecture at four different time-points: 0:22, 6:53, 16:18, and 20:31. The tumor temperatures and RF power at those time points are shown in the upper-middle and upper-right hand side sections, respectively. (6E) illustrates the change in temperature and power with respect to time. Vessel degradation can be seen for temperatures>41° C. A complete breakdown of the vessel architecture can be seen for temperatures>47° C.

FIGS. 7A-7I illustrate an example of an embodiment of the use of RF Multi-Channel IVM-RF imaging. (7A) Overlay of the independent IVM channels (FITC, Texas Red, and Cy5). (7B) Tumor vessels are highlighted using FITC-dextran fluorescent tracers, (7C) Fluorescent emission from the transfected 4T1 tumor cell line, (D) Cy5 emission from the DiD-stained Red Blood Cells. (7A-7D) were taken at time=78 s. (7E-7H) depict the FITC channel (vessels) at different time points: 762, 1650, 2382, and 2742 s, respectively. (7I) illustrates the tumor temperature with respect to time and applied RF power. The numbers 1-5 shown in the bottom left hand side of each figure correspond to the 5 different time-points highlighted in (7I).

FIGS. 8A-8I demonstrate that RF exposure enhances transport of fluorescent-bound albumin (MW˜66 kDa) across transport barrier into tumor region. (8A and 8B) depict the blue image channel only (albumin) before and after (4.5 min) RF exposure. This data is shown superimposed with the tumor channel in (8C and 8D). (8E) Control mouse (no RF) was imaged for 30 minutes on both channels. As can be seen, there is no transport or perfusion of albumin into the tumor across the transport barrier. (8F) Time lapsed images of the data shown in (8A and 8B). (8G) 4T1 tumor slices immunohistologically stained to the antibodies CD31 (green, vasculature endothelial cells), and albumin (red) for both RF (G1) and non-RF (G2) groups. (8H) depicts positive area fraction of albumin accumulation in tumor slices. Finally, (8I) is a qualitative video analysis of increase in albumin fluorescence in multiple 4T1 tumor surfaces exposed to RF under IVM (n=3).

FIGS. 9A-9D shows post-RF analysis of as-captured IVM images using template and masking algorithms. FITCDextran was injected with 30 mins RF exposure. Albumin was then injected and imaged for 30 minutes without RF exposure. (9A) The tumor area (i) is demarcated using a green line and allows inter-tumoral FITC-dextran (ii) and albumin (iii) perfusion to be monitored. (9B) These masks are applied to the full timelapsed video for all channels (RBG). (9C) Areas where both albumin and FITC-dextran overlap are processed to quantify the relative average intensity of enhanced albumin perfusion after 30 mins of RF exposure. (9D) Enhanced perfusion of FITC-dextran is seen for the intital 30 mins of RF exposure. The RF is then turned off and albumin injected into mouse. Enhanced albumin perfusion is evident after RF exposure suggesting RF-mediated effects are long-lived.

FIGS. 10A-10D provide PANC-1 orthotopic tumor slices immunohistologically stained to the antibodies CD31 (red, vasculature endothelial cells), and albumin (green) for both RF (10A) and non-RF (10B). (10C) depicts positive area fraction of dextran accumulation in tumor slices. (10D) depicts thermal optic probe heating data of tumor region and skin.

FIGS. 11A-11E illustrate an example of an embodiment of the use RF energy to enhance the uptake of chemotherapy drugs into tumors compared to using chemotherapy drugs alone. (11A) Female Nude mice (4-6 weeks old) were given direct injections of KPC pancreatic adenocarcinoma cells (1×10⁶) into the pancreas. After 1-2 weeks (enough time for a orthotopic pancreatic tumor to form) the mice were subjected to RF exposure under a ramped power treatment protocol as shown in the table. (11B) Graph of a typical RF power and resulting temperature versus time plot for a single mouse being exposed to RF energy. The surface temperature of the mouse in close proximity to the pancreas was recorded using an IR camera. The RF exposure was turned off once the temperature reached 41° C. (11C) IR camera image of the mouse at the end of the treatment. (11D) Example of enhanced uptake of Gemcitabine. Six mice with orthotopic pancreatic tumors were given systemic injections of Gemcitabine at a dosage of 70 mg/Kg. Three of the mice were immediately exposed to RF conditions shown in (11B) and were then sacrificed 5 hours later and compared to the non-RF treated mice, also sacrificed 5 hours after administration of Gemcitabine. There is approximately 4× more Gemcitabine present in the tumors of the RF-treated mice than the control group indicating RF exposure enhances uptake of chemotherapeutics into tumors. Quantification of Gemcitabine was analyzed using Liquid Chromatography Mass Spectroscopy (LC-MS). (11E) Quantification of the levels of Gemcitabine in the orthotopic pancreatic tumors of mice with and without RF exposure. Total tumor weights are also shown and are approximately equal in size/mass.

FIG. 12 illustrates an example of an embodiment of the use RF energy to enhance the uptake of chemotherapy drugs into tumors compared to using chemotherapy drugs alone. Abraxane intravenous injections were given to nude mice bearing orthotopic 4T1 breast tumors. Dosage was 125 mg/kg. Tumors were harvested at 30 mins and 5 hrs after injection, with and without RF exposure (conditions similar to that described in FIG. 11B). The active ingredient in abraxane, Paclitaxol, was analyzed using LC-MS. As can be seen, there is clear evidence of enhanced retention of paclitaxel in the tumors even for 5 hours post-RF exposure. The enhanced retention observed at 5 hours may be due to high-temperature vessel degradation and coagulation, which would lock the drugs inside the tumor preventing them from leaving the tumor micro environment.

FIGS. 13A-13D illustrate an example of an embodiment of the use RF energy to provide synergy between RF exposure and chemotherapeutics such as Gemcitabine. (13A) Mice bearing subcutaneous pancreatic tumors were exposed to either RF, Gemcitabine, or RF+Gemcitabine once per week until the control group tumor size became a burden (n=5 in each group). The RF system used can be seen in (13B) and is a higher power version (0-1200 W) of the portable RF system. RF conditions were similar to that shown in (13B). Gemcitabine was given intravenously at a dosage of 70 mg/Kg. As can be seen in (13A) there is a clear reduction in tumor size in the Gemcitabine+RF group when compared to the other groups indicating the synergistic effects of RF on the efficacy of chemotherapeutics. (13C) The experiment was repeated using a portable RF probe system (smaller power, 200 W), which is shown in (13D). As can be seen a similar synergy effect between Gemcitabine and RF is also evident even when using a lower power system. The mice in the portable RF system experiment were exposed to 200 W of RF power for 15 minutes.

FIGS. 14A-14B shows the effect of hyperthermia on HRR-pathway proteins. Three liver cancer cell lines were subjected to hyperthermia at 42.5° C. for two hours and levels of proteins monitored over time. (14A) Immunoblots. (14B) Quantification by densitometry. Data are mean absorbance units (a.u.) and error bars represent the standard deviation (SD) from three independent experiments with three replicates. *P<0.05 by the Student unpaired two-sided t test in comparison with control group. Also see FIG. 21. C=control, before hyperthermia; HT=hyperthermia.

FIGS. 15A-15D provide inhibition of Mre11 and Rad51 recruitment at gemcitabine-stalled replication forks by hyperthermia. (15A) Hep3B cells were exposed to hyperthermia and/or gemcitabine. (15B) and (15C) Mre11 and Rad51 were recruited at gemcitabine-stalled replication forks. Cells treated with hyperthermia or hyperthermia with gemcitabine demonstrated decreased colocalization of Mre11 and Rad51 with γ-H2AX foci. Scale bar=10 μm. (15D) Quantification of data from colocalization experiments. Data are mean Pearson correlation (a.u.) and error bars represent the SD from three independent experiments with 10 replicates each (*P<0.05, **P<0.01). Gem=Gemcitabine; HT=Hyperthermia; N/A=not applicable, i.e., in hyperthermia treated cells BrdU staining is not detected as distinct foci and therefore correlation could not be quantified; ns=not significant, by the Student unpaired two-sided t test in comparison with gemcitabine alone group. Also, see FIGS. 22-24.

FIGS. 16A-16D demonstrate hyperthermia inhibition of postreplication recombination repair at gemcitabine-stalled replication forks. (16A) Experimental scheme. (16B) Cell cycle progression was monitored using flow cytometry in Hep3B cells after release from gemcitabine-induced G1/S arrest. (16C) Median DNA content of Hep3B cells was quantified using flow cytometry after treating them according to the design in (16A). (Data points: means of median DNA content; error bars represent SD from three independent experiments with 10 000 counted events per experiment. Gem vs. Gem+HT, *P<0.05 by the Student unpaired two-sided t test at the specified time point). (16D) Hep3B cells were treated with gemcitabine+/−hyperthermia and cells positive for γ-H2AX foci in confocal microscopy images were quantified. (Data points: mean percent of γ-H2AX foci positive cells; error bars represent SD from three independent experiments with at least 10 replicates each. Gem vs. Gem+HT, *P<0.05 by the Student unpaired two-sided t test at the specified time point). Gem=Gemcitabine; HT=Hyperthermia; t=Time.

FIGS. 17A-17E demonstrates hyperthermia sensitization of hepatocellular carcinoma cells to gemcitabine in a dose-dependent manner. (17A) Experimental scheme. (17B and 17D) Experimental design i, ii, and iii were used. (17C and 17E) Experimental design iv was used. Data points represent mean surviving fraction, and error bars represent SD from three independent experiments, *P<0.05 by the Student unpaired two-sided t test in comparison with control group. Gem=Gemcitabine; HT=Hyperthermia.

FIGS. 18A-18B shows hyperthermia-induced gemcitabine sensitivity of hepatocellular carcinoma cells through an Mre11-dependent pathway. (18A) Hep3B cells were treated with gemcitabine alone (Gem), hyperthermia+/−gemcitabine (HT), Mirin+/−gemcitabine (Mirin) or Hyperthermia and Mirin+/−gemcitabine (Mirin-HT). In all cases hyperthermia followed gemcitabine or mirin exposure. (18B) Control (shControl) or Mre11 knockdown (shMre11) were treated with or without gemcitabine followed by +/−hyperthermia. Data points represent mean surviving fraction and error bars represent SD from three independent experiments, *P<0.05 by the Student unpaired two-sided t test in comparison with control group. Gem=Gemcitabine; HT=Hyperthermia.

FIGS. 19A-19B shows efficacy of gemcitabine and non-invasive RF combination therapy in mice bearing orthotopic hepatocellular carcinoma xenografts. Tumor weight and percent growth inhibition are shown for Hep3B (19A) and HepG2 (19B) xenografts. Data points represent mean tumor mass (mg) and error bars represent SD, n=7-10, *P<0.05, ***P<0.001 vs gemcitabine by the Student unpaired two-sided t test.RF=Radiofrequency. Also, see FIGS. 25-30.

FIGS. 20A-20C shows an example of radiofrequency generator and fiber optic probe placement. (20A) For fiberoptic thermography, a temperature-sensing probe is placed over a 20G needle. The needle is advanced into the tumor (T) under ultrasound guidance. The probe is then advanced over the needle and the needle is withdrawn. (20B) A 13.56 MHz external RF generator system is shown (black box) that is connected to an end-firing antenna in the transmission head (TX). A spacing of 3.5 inches exists between the Tx head and the receiver head (RX)/ground plate. (20C) A CB17 SCID mouse is placed supine on the RX head.

FIG. 21 shows an effect of moderate hyperthermia on HRR-pathway proteins. Three liver cancer cell lines were subjected to hyperthermia at 42.5° C. for 2 hours and levels of proteins were monitored over time by western immunoblot. C=control, before hyperthermia.

FIG. 22 shows that Gemcitabine-stalled replication forks are marked by γ-H2AX foci. Hep3B cells were exposed to hyperthermia and/ or gemcitabine and DNA immediately downstream of stalled replication forks was labeled with BrdU. Distinct foci marking gemcitabine-stalled replication forks are detected by anti-BrdU antibody. γ-H2AX foci colocalize at sites of stalled replication.

FIGS. 23A-23B demonstrates that hyperthermia-induced γ-H2AX foci are S-phase specific. (23A) S-phase cells were marked using BrdU incorporation over a period of 4.5 hours immediately after hyperthermia. (23B) It was noted that hyperthermia-induced γ-H2AX foci only occurred in cells staining positive for BrdU (S-phase cells).

FIGS. 24A-24B show that gemcitabine and hyperthermia-stalled replication forks recruit RPA. (24A) Hep3B cells were exposed to hyperthermia and/or gemcitabine; 24B. RPA colocalizes with γ-H2AX foci in cells treated with gemcitabine, hyperthermia or combination of the two treatments.

FIGS. 25A-25F illustrate animal model characterization. (25A) A Hep3B xenograft is seen in the left lobe of CB17 SCID mouse liver at the time of necropsy. (25B) The development of luciferase expressing Hep3B or HepG2 xenografts can be tracked using bioluminescence imaging. (25C-25F) Histological analysis demonstrates that these xenografts mimic human hepatocellular carcinoma based on growth pattern, hyper-vascularity, erosion and spontaneous central necrosis.

FIGS. 26A-26D demonstrate thermal dose quantification in Hep3B xenografts under RF field exposure (13.56 MHz, 600 W). (26A) Xenograft and abdominal surface temperatures were measured in real-time using fiber optic thermography and infrared thermography, respectively in tumor-bearing mice. (26B) Similar measurements were performed on the normal livers of non-tumor-bearing mice. (26C) Data in (26A and 26B) are combined for comparative representation. Abdominal surface temperature is the composite average from tumor and non-tumor-bearing mice. (26D) Tumor and surface temperature from (26A) is plotted and was found to correlate in a linear fashion.

FIGS. 27A-27C show efficacy of gemcitabine and Mirin combination therapy in mice bearing Hep3B xenografts. Tumor weight, percent growth inhibition and macroscopic appearance are represented in (27A, 27B and 27C), respectively.

FIG. 28 shows inhibition of Mre11 localization to stalled forks in vivo. Hep3B xenografts were evaluated for colocalization of γ-H2AX and Mre11 foci. As shown, non-invasive RF exposure inhibits Mre11 localization to stalled replication forks.

FIG. 29 demonstrates inhibition of Rad51 localization to stalled forks in vivo. Hep3B xenografts were evaluated for colocalization of γ-H2AX and Rad51 foci. As shown RF exposure inhibits Rad51 localization to stalled replication forks.

FIGS. 30A-30B show that RF exposure inhibits resolution of gemcitabine-induced DNA damage in Hep3B xenografts. (30A) Sites of DNA damage were evaluated using fluorescence immunohistochemistry for γ-H2AX. Also note the increased nuclear fragmentation in the DAPI channel for combination therapy groups. (30B) Tumors treated with combination therapy demonstrate significantly higher γ-H2AX staining. *p<0.05, **p<0.01 vs. gemcitabine.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method or composition described herein can be implemented with respect to any other method or composition described herein.

The term “chemoresistance” or “chemoresistant” or “resistance to cancer drug” and the like is defined herein as the resistance of malignant cells to the inhibition action of one or more chemical compounds used in treatment. The term, as contemplated herein, may refer to partial resistance, wherein the cancer cells may be killed but only with higher or excessive doses of the drug, yet it may also refer to complete resistance, wherein cancer cells are no longer being killed with the drug.

The term “non-invasive” as used herein refers to when there are no needles, wires, electrodes, or other objects that are inserted into an individual or tumors of an individual to be treated.

The term “radiofrequency absorption enhancer” as used herein refers to a composition that increases the tendency of a specific target area to absorb more energy from an radiofrequency signal.

### I. General Embodiments

As used herein, “individual” or “subject” may be used interchangeably and refers to a mammal including humans, primates, farm and laboratory animals, and domesticated pets.

The differential dielectric properties of cancerous and normal tissues cause radiofrequency energy to be selectively absorbed and converted to heat to a greater extent in tumors. This heating is because of larger dielectric losses within tumor tissue compared to normal tissues. Exploitation of such a distinction provides a basis for embodiments of the disclosure. In specific embodiments, radiofrequency energy is employed as part of a bipartite cancer regimen to treat cancer, including, for example, cancer that is resistant to at least one type of therapy. In particular cases, the radiofrequency is effective through production of hyperthermia at a targeted site. In specific embodiments, the cancer treatment system of the disclosure allows for imaging of the dynamic interplay between low power radiofrequency electric-fields and biological tissue in vivo and in real-time at high-resolution. Radiofrequency exposure enhances tumor perfusion of systemically administered blood-circulating macromolecules such as cancer drugs. Not wishing to be bound by particular theory, in distinctive embodiments, it is believed that enhanced perfusion is observed because of increased intra-tumoral blood vessel permeability: the increased permeability is in addition to what already exists in the compromised leaky tumor vasculature, inducing a state of hyperpermeability.

In specific embodiments, the radiofrequency therapy is effective for those drugs that impact, directly or indirectly, one or more processes in the nucleus, and in at least certain embodiments such efficacy is related to the hyperthermia generated by the non-invasive radiofrequency therapy that inhibits recombination repair of replication forks stalled by the drug itself. Thus, in specific embodiments, there is provided a method of preventing or inhibiting recombination repair of a drug-stalled replication fork comprising the step of exposing cells with the replication forks stalled by a drug to radiofrequency that then induces hyperthermia; the hyperthermia inhibits homologous recombination repair pathway(s) of the drug-stalled replication forks, in specific embodiments.

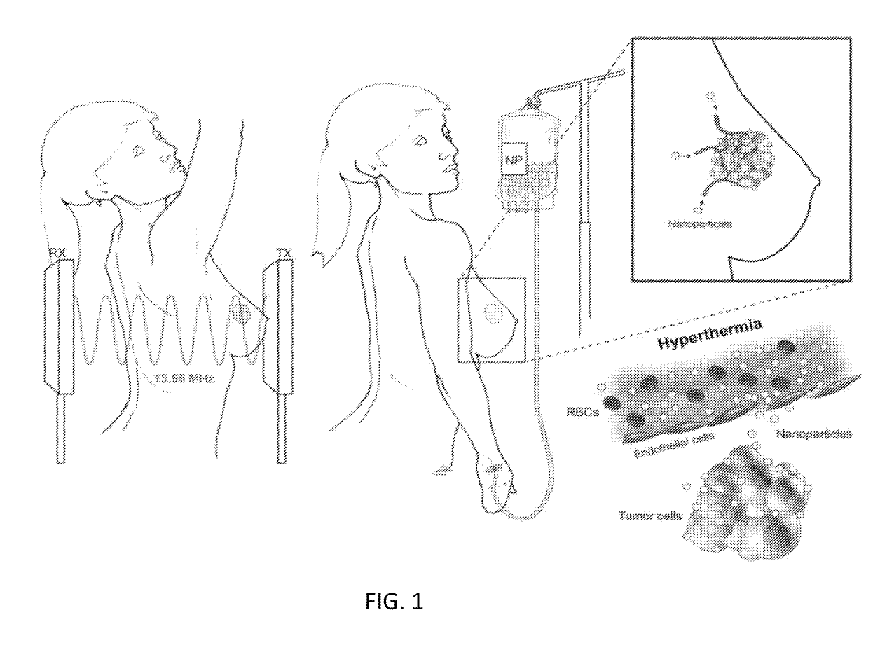

Although applicable to other solid tumors, a schematic of an example of clinical non-invasive RF protocol for patients with breast cancer (merely as an example) is presented in FIG. 1. The patient would be situated between two RF electrodes—one the transmitter (TX) and the other the receiver (RX). The frequency of operation, 13.56 MHz, is a medically approved frequency and is even one of the main operating frequencies of RF coils in magnetic resonance imaging machines. Under operation, the system power is increased incrementally until biological temperature responses are evident. Given the correct RF thermal dose, tissues and organs would slowly heat. However, because of the physical difference in electronic and fluidic properties between cancerous and normal tissues, the tumors heat at a much greater rate: reaching hyperthermia before healthy tissues. This is different from the currently used clinical method of invasive radiofrequency ablation (RFA), which is used invasively through insertion of an electrode into the tissue ablating both primary and metastatic hepatic carcinomas, leaving behind cancer debris, coagulation necrosis, and damaging adjacent normal tissue.

In particular embodiments, non-invasive radiowaves induce hyperthermia in tissues with elevated temperature increases in tumor tissue compared to normal tissue, based on the unique properties of the tumor. An individual is positioned between a transmitting head and a receiving head, and a generator creates high-frequency radiowaves (13.56 MHz) that pass through the individual's body non-invasively. Absorption of the energy generates heat that targets susceptible cancer cells. The heat increases blood flow and accumulation of nanoparticles and macromolecules (for example) in the tumor, and in turn the nanoparticles/drugs pre-sensitize cancer cells to RF therapy. In particular embodiments, methods of the disclosure enhance the therapeutic efficacy of chemotherapeutics and/or nanotherapeutics, enabling the use of less toxic doses of the composition(s) by increasing accumulation and cellular uptake in the tumor and increasing effectiveness.

### II. Radiofrequency Therapy Embodiments

In particular embodiments of the disclosure, non-invasive radiofrequency therapy is employed as part of a multi-part approach to cancer treatment using cancer drugs, to which cancer cells are known to be or become or are suspected of becoming chemoresistant. In other embodiments, however, the non-invasive radiofrequency therapy is used in conjunction with cancer treatment(s) against cancer that are not, or are not susceptible to being, chemoresistant. The methods of the present disclosure may be utilized for cancer that is or is not chemoresistant.

Some methods of employing non-invasive radiofrequency therapy for directed cancer therapy are known in the art, and the parameters for the non-invasive radiofrequency therapy may be optimized by the skilled artisan (for examples, see U.S. Pat. Nos. 7,510,555 and 7,627,381, both of which are incorporated by reference herein in their entirety). In specific embodiments, the methods of the present disclosure specifically do not employ one or more compositions specifically selected to be non-invasive radiofrequency absorption enhancers.

Embodiments of the disclosure include providing to an individual in need thereof radiofrequency under sufficient conditions to generate heat to kill or damage target cancer cells by heat generated by the application of a non-invasive radiofrequency field, such as one generated by a radiofrequency signal between a transmission head and a reception head that is different from the transmission head. One can configure the transmission and reception heads on opposite sides of a desired target of the individual for treatment (such as a tumor site(s) or the whole body) and irradiate the site(s) between the transmission and reception heads with a radiofrequency field to kill or damage the target cells from the interaction of the radiofrequency field with the cancer cells.

In specific embodiments, a non-invasive radiofrequency therapy system comprises a radiofrequency transmitter in communication with a transmission head and a radiofrequency receiver in communication with a reception head. The communication may be direct electrical, optical, and electromagnetic connections and indirect electrical, optical, and electromagnetic connections. That is, two devices are in communication if a signal from one is received by the other, regardless of whether the signal is modified by some other device. In an example of a non-invasive radiofrequency therapy system, the radiofrequency transmitter generates a radiofrequency signal at a frequency for transmission via the transmission head. In some cases, the radiofrequency transmitter has controls for adjusting the frequency and/or power of the generated radiofrequency signal and/or may have a mode in which a radiofrequency signal at a predetermined frequency and power are transmitted via the transmission head. In some cases, the radiofrequency transmitter provides a radiofrequency signal with variable amplitudes, pulsed amplitudes, multiple frequencies, etc.

In particular embodiments, the radiofrequency receiver is in communication with the reception head and is tuned such that at least a portion of the reception head is resonant at the frequency of a radiofrequency signal transmitted via the transmission head. As a result, the reception head receives a radiofrequency signal that is transmitted via the transmission head. In specific cases, the transmission head and reception head are arranged proximate to and on either side of a general target area, such as an area that has the tumor to be treated. The transmission head and reception head may be insulated from direct contact with the general target area, in certain aspects. In specific cases, the transmission head and reception head are insulated by means of an air gap, although in some cases it is an insulating layer or material, such as, for example, Teflon®. One can include an insulation area on the heads, allowing the heads to be put in direct contact with the general target area. The transmission head and the reception head may include one or more plates of electrically conductive material such as gold, silver, or copper.

The target tumor absorbs energy through its inherent dielectric and electrical properties and is warmed as the radiofrequency signal travels through the target tumor area that is desired to be treated by inducing hyperthermia. The more energy that is absorbed by an area, the higher the temperature increase in the area. In specific embodiments, the target area is heated to between 37° C.-45° C., for example. Energy absorption in the target tumor area can be increased by increasing the radiofrequency signal strength, which increases the amount of energy traveling through the area. One method of inducing a higher temperature in a specific target tumor area includes using a reception head that is smaller than the transmission head. The smaller reception head picks up more energy due to the use of a high-Q resonant circuit. In specific embodiments, the temperature is monitored, for example by MRI thermography.

In specific embodiments, the radiofrequency power is determined by the type of system being employed. For example, for a portable system one may utilize 0-200 watts (W). In particular cases wherein the system is not portable, one may employ, e.g., from 700 W-1500 W to maintain a localized electric-field of strength 0-90 kV/m.

In certain embodiments, one or more particular wavelengths are employed. In specific cases, a frequency of 13.56 MHz is employed. Other examples are 1 MHz, 6.78 MHz, 8 MHz, 27.12 MHz, 40.68 MHz, 128 MHz, etc. In specific embodiments, a frequency range of 100 kHz to 1 GHz is employed. Other examples of ranges include 250 kHz-1 GHz, 500 kHz-1 GHz, 1000 kHz-1 GHz, 10,000 kHz-1 GHz, 100,000 kHz-1 GHz, 1 MHz-1 GHz, 10 MHz-1 GHz, 100 MHz-1 GHz, 500 MHz-1 GHz, 10 MHz-50 MHz, 10 MHz-100 MHz, 10 MHz-250 MHz, 10 MHz-500 MHz, and so forth.

The frequency and duration of exposure of the non-invasive radiofrequency therapy to the individual may be optimized for the individual, type of cancer, gender, size of the individual, and so forth. In specific embodiments, the individual may be provided with the non-invasive radiofrequency therapy and optionally one or more cancer drugs once or more than once during a particular period of treatment. In specific embodiments, the radiofrequency therapy and cancer drug are provided to the individual over the course of 1-7, 1-6, 1-5, 1-4, 1-3, 1-2, 2-7, 2-6, 2-5, 2-4, 2-3, 3-7, 3-6, 3-5, 3-4, 4-7, 4-6, 4-5, 5-7, 5-6, or 6-7 days or over the course of 1-4, 1-3, 1-2, 2-4, 2-3, or 3-4 weeks. However, in some cases the radiofrequency therapy and cancer drug are provided to the individual over the course of 1-12, 1-11, 1-10, 1-9, 1-8, 1-7, 1-6, 1-5, 1-4, 1-3, 1-2, 2-12, 2-11, 2-10, 2-9, 2-8, 2-7, 2-6, 2-5, 2-4, 2-3, 3-12, 3-11, 3-10, 3-9, 3-8, 3-7, 3-6, 3-5, 3-4, 4-12, 4-11, 4-10, 4-9, 4-8, 4-7, 4-6, 4-5, 5-12, 5-11, 5-10, 5-9, 5-8, 5-7, 5-6, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 months. An individual may be provided the non-invasive radiofrequency therapy upon recurrence of a cancer from remission or upon having another type of cancer altogether, in which case the combination radiofrequency/cancer drug deliveries may be employed years apart.

The duration of exposure to radiofrequency may be of any suitable time, but in specific embodiments, it is on the order of minutes. In particular cases, the duration of exposure of the radiofrequency therapy for the individual is between 5-10, 5-9, 5-8, 5-7, 5-6, 6-10, 6-10, 6-8, 6-7, 7-10, 7-9, 7-8, 8-10, 8-9, or 9-10 minutes. In cases wherein multiple exposures are provided to an individual, the different exposures may or may not be for the same duration in time.

In specific embodiments, the non-invasive radiofrequency therapy and cancer drug are provided to the individual once a day or more than once a day. The non-invasive radiofrequency therapy and cancer drug may be provided to the individual once a week or more than once a week. The non-invasive radiofrequency therapy and cancer drug may be provided to the individual over the course of weeks or months. In cases wherein there are multiple deliveries of the cancer drug as part of the combination with non-invasive radiofrequency therapy, one or more of the multiple deliveries may be of a cancer drug that is not the cancer drug that was initially employed in the combination therapy.

In particular embodiments, the temperature to substantially damage the targeted tumor cells is sufficient to kill the tumor cells without damaging or substantially damaging surrounding normal cells and without tissue burn, for example. In specific embodiments, the temperature range that the tumor cells are heated is between 37° C. and 45° C., as an example.

In particular embodiments of the disclosure, the methods of the disclosure comprise, consist of, or consist essentially of providing radiofrequency therapy and a cancer drug to the individual. In specific embodiments, the methods do not employ a non-invasive radiofrequency absorption enhancer (such as gold nanoparticles and/or carbon nanotubes), including a non-invasive radiofrequency absorption enhancer that was specifically selected to act as a non-invasive radiofrequency absorption enhancer in the methods for the purpose of acting as a radiofrequency absorption enhancer.

### III. Chemoresistant Cancer Drugs

Embodiments of the disclosure include the use of non-invasive radiofrequency therapy with one or more cancer drugs. The individual to be treated or being treated for cancer may or may not be resistant to the one or more cancer drugs. Certain embodiments of the disclosure provide for methods for treating cancer with one or more cancer drugs or for overcoming resistance to one or more cancer drugs in an individual by using radiofrequency therapy in conjunction with the cancer drug(s). Such a combination results in a synergistic effect for the cancer, in specific embodiments. Not wishing to be bound by a particular theory, it is believed that in specific aspects, the cancer drug(s) acts in the nucleus in synergism with the radiofrequency therapy that induces hyperthermia resulting in modulation of homologous recombination repair systems in the nucleus. The combination of cancer drug(s) and radiotherapy may be provided to the individual for the purpose of modulating one or more homologous recombination repair systems in the nucleus. Thus, the cancer drug acting in the nucleus may be, for example, one of an alkylating agent, antimetabolite, anthracycline, or topoisomerase inhibitor, as examples and combinations thereof. Specific exemplary cancer drugs that may be administered in accordance with the present invention include but are not limited to gemcitabine; abraxane; cetuximab; nitrogen mustards, such as mechlorethamine (nitrogen mustard), chlorambucil, cyclophosphamide (Cytoxan®), ifosfamide; melphalan; nitrosoureas, such as streptozocin, carmustine (BCNU), and lomustine; alkyl sulfonates, such as busulfan; triazines, such as dacarbazine (DTIC) and temozolomide (Temodar®); ethylenimines, such as thiotepa and altretamine (hexamethylmelamine); 5-fluorouracil (5-FU); 6-mercaptopurine (6-MP); Capecitabine (Xeloda®); Cladribine; Clofarabine; Cytarabine (Ara-C®); Floxuridine; Fludarabine; Gemcitabine (Gemzar®); Hydroxyurea; Methotrexate; Pemetrexed (Alimta®); Pentostatin; Thioguanine; Daunorubicin; Doxorubicin (Adriamycin®); Epirubicin; Idarubicin;topotecan; irinotecan; and a combination thereof. In alternative embodiments the cancer drug for the cancer is utilized for a cancer that is chemosensitive. In alternative embodiments, the cancer drug acts in the cytoplasm and not in the nucleus.

The individual may or may not be resistant to the cancer drug. The individual may show resistant to the cancer drug initially or early on in the treatment cycle or may develop resistance to the cancer drug over time. All of these potential outcomes may be treated by methods of the present disclosure.

### IV. Pharmaceutical Preparations of Cancer Drugs

Pharmaceutical compositions of the present disclosure comprise an effective amount of one or more cancer drugs dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to an animal, such as, for example, a human, as appropriate, and do not interfere with the therapeutic methods of the disclosure. The preparation of an pharmaceutical composition that contains at least one cancer drugs or additional active ingredient will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The cancer drug may comprise different types of carriers depending on whether it is to be administered in solid, liquid or aerosol form, and whether it needs to be sterile for such routes of administration, such as injection. The cancer drugs of the present disclosure can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The cancer drug may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in practicing the methods of the present disclosure is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may include the use of a pharmaceutical lipid vehicle compositions that incorporates a cancer drug, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the cancer drug may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to the subject can be determined by physical and physiological factors such as body weight, severity of condition, the type of disease being treated, previous or concurrent therapeutic interventions, idiopathy of the subject and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% (by weight) of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration of the active agent, e.g., a cancer drug according to the present disclosure, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., of the active agent can be administered, based on the numbers described above.

Alimentary Compositions and Formulations

In particular embodiments of the present disclosure, the cancer drug is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10% (by weight), and preferably about 1% to about 2% (by weight).

Parenteral Compositions and Formulations

In further embodiments, the cancer drug may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof A powdered composition is combined with a liquid carrier such as, e.g., water or a saline solution, with or without a stabilizing agent.

Miscellaneous Pharmaceutical Compositions and Formulations

In other preferred embodiments of the disclosure, the active compound cancer drug may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Kits Comprising the Cancer Drug

Any of the cancer drugs described herein may be part of a kit. The kits may comprise a suitably aliquoted cancer drug of the present disclosure, and the component(s) of the kits may be packaged either in aqueous media or in lyophilized form. The container of the kits will generally include at least one vial, test tube, flask, bottle, syringe or other container, into which a component may be placed, and preferably, suitably aliquoted. Where there is more than one component in the kit, the kit also will generally contain a second, third or other additional container into which the additional component(s) may be separately placed. However, various combinations of components may be comprised in a vial. The kits of the present disclosure also will typically include container for holding the cancer drug and any other reagent containers in close confinement for commercial sale.

When the components of the kit are provided in one and/or more liquid solutions, the liquid solution is an aqueous solution, with a sterile aqueous solution being contemplated. The compositions may also be formulated into a syringeable composition. In which case, the container may itself be a syringe, pipette, and/or other such like apparatus, from which the formulation may be applied to a particular area of the body, injected into an individual, and/or even applied to and/or mixed with the other components of the kit. However, the component(s) of the kit may be provided as dried powder(s). When reagents and/or components are provided as a dry powder, the powder can be reconstituted by the addition of a suitable solvent. It is envisioned that the solvent may also be provided in another container.

### V. Therapeutic Embodiments

Embodiments of the disclosure provide for therapeutic treatment of cancer in an individual, including for cancer that is resistant to one or more cancer drugs, although in some cases the cancer is not resistant to the cancer drug. In specific embodiments, an individual is being treated with the drug or will be treated with the drug, and the individual is provided the combination radiofrequency therapy and cancer drug.

An effective combination of the non-invasive radiofrequency therapy and the cancer drug may occur when the two components are provided to the individual in need thereof at the same time or at different times. In either case, multiple rounds of the combination therapy may be provided to the individual. When the radiofrequency therapy and the cancer drug are provided to the individual at different times, the radiofrequency therapy may precede and/or follow the cancer drug. When they are given at separate times, they may do so within the span of seconds, minutes, hours, days, weeks, or months. In specific embodiments, they are provided to the individual within 1, 2, 3, 4, 5, 6, 7, 8, 9, 10, 15, 20, 30, 40, 50, or 60 or more minutes of each other or within 1, 2, 3, 4, 5, 6, 7, 8, 9, 10, 11, 12, 15, 20, or 24 or more hours of each other. In addition, during the course of the combined therapy one or more rounds of either RF or chemotherapy may be skipped as may be deemed appropriate by a medical professional.

The cancer drug may be given by any suitable route, but in specific embodiments, the cancer drug is provided such that it is present in the bloodstream. Thus, in particular aspects the drug is delivered systemically, such as intravenously, for example.

Identification of the area to be treated in the individual may encompass any suitable means for determining location of one or more tumors. In specific embodiments, the area to be treated may be identified by palpatation, x-ray, endoscopy, magnetic resonance imaging, CT scan, radionuclide scan, positron emission tomography scan, and/or ultrasound, for example. In particular embodiments, one can image the area of interest such that the radiofrequency therapy may be focused accurately on the tumor.

In specific cases, the cancer being treated comprises solid tumors, although cancers such as leukemia and lymphoma may be treated as well (such as utilizing whole body exposure of the radiofrequency waves). In specific cases, the solid tumor being treated may be as small as 1 centimeter although any tumor or cancer that is detectable can be treated, even on a single cellular level.

Any type of cancer may be treated with methods of the disclosure, including pancreatic, liver, breast, brain, lung, colon, kidney, stomach, testicular, ovarian, skin, bone, gall bladder, spleen, thyroid, prostate, peritoneal carcinomatosis, sarcomas, and so forth. In specific embodiments, the cancer is chemoresistant, although in alternative embodiments it may be chemosensitive.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A New Imaging Platform for Visualizing Enhanced Macromolecule Perfusion into Tumors Using Non-Invasive Radiofrequency Cancer Therapy

Nude mice (4-6 wk old) were obtained from Charles River Laboratories, Inc. (Wilmington, Mass.). Breast tumors were established using fluorescent 4T1 td-Tomato Bioware® Ultra Red mouse mammary cancer cells purchased from Caliper Life Sciences (Hopkinton, Mass.). Palpable tumors were used once reaching a size ˜5-7 mm in diameter. Pancreatic PANC-1 tumors were generated by an orthotopic injection of 5×10⁵ cells (ATCC, Manassas, Va.) into the pancreas of nude mice (ages 4 to 6 weeks). Palpable tumors were used once reaching a size ˜5-7 mm in diameter (250-350 mm³). Animals were euthanized via CO₂ exposure followed by cervical dislocation. All procedures were performed in accordance with protocols approved by the Institutional Animal Care and Use Committee at Baylor College of Medicine and Houston Methodist Research Institute and according to the NIH Guide for the Care and Use of Laboratory Animals.

Portable RF System

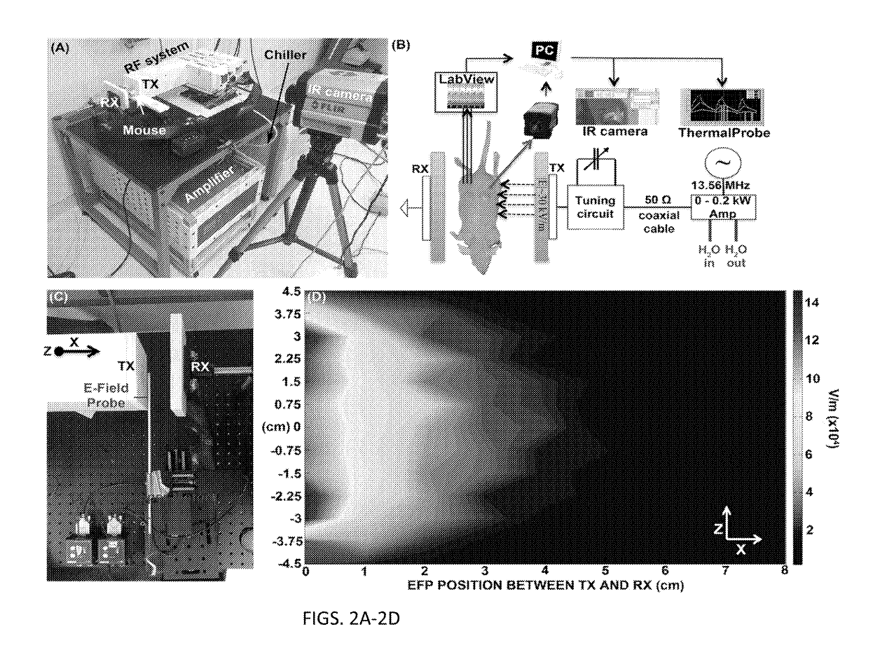

A photograph of the portable-RF system (p-RF) system alongside a schematic representation of the p-RF experimental setup is illustrated in FIG. 2. The device is powered by a 200 W fixed-frequency (13.56 MHz) water-cooled power supply (Seren, RX01/LX01 Series, Industrial Power Systems, Inc), which is connected via a high-current carrying capacity 50 Ω co-axial cable. The sample (i.e. mouse, quartz cuvette, etc.) to be exposed to RF is placed between the transmitting and receiving head (TX and RX, respectively). Increases in temperature are recorded either through the use of 1 mm outer diameter fiber optic Teflon coated thermal probes (Photon Control, Canada) with a temperature accuracy of ±0.5° C. or by an infrared (IR) camera (FLIR SC 6000, FLIR Systems, Inc., Boston, Mass.) with a temperature accuracy of ±2° C. (640×512 resolution InSb detector with a mid-wavelength IR spectral range of 3.0-5.0 μm). Thermal probe data was captured using a custom built LabVIEW Virtual Instrument (National Instruments, Austin, Tex.). The generated RF electric-field was characterized using a Teflon coated electric field probe (TherMed, LLC, Erie, Pa.) attached to an adjustable x,y,z stage (Thorlabs, Inc.) for accurate special positioning. This is shown in FIG. 2C and FIG. 2D. As can be seen in FIG. 2D, the ‘active’ area of RF electric-field exposure is centered ˜6 cm around the mid-point of the TX head and extends ˜1-2 cm across the x-axis. As will be seen through this work, this causes a heating profile that is gradually reduced as the sample is located further away from the TX head. The design delivers a strong alternating (13.56 MHz) electric field across the TX and RX heads using a cascade LC network. However, unlike our previous systems, this system is not capacitively coupled and does not model an ideal parallel-plate capacitor configuration where the electric field would be approximately uniform across the TX, RX heads. Instead, this system transmits an electric-field which gradually diminishes across the TX-RX heads and is hence classified as “end fired transmission configuration”.

Tumor Temperature Modulation

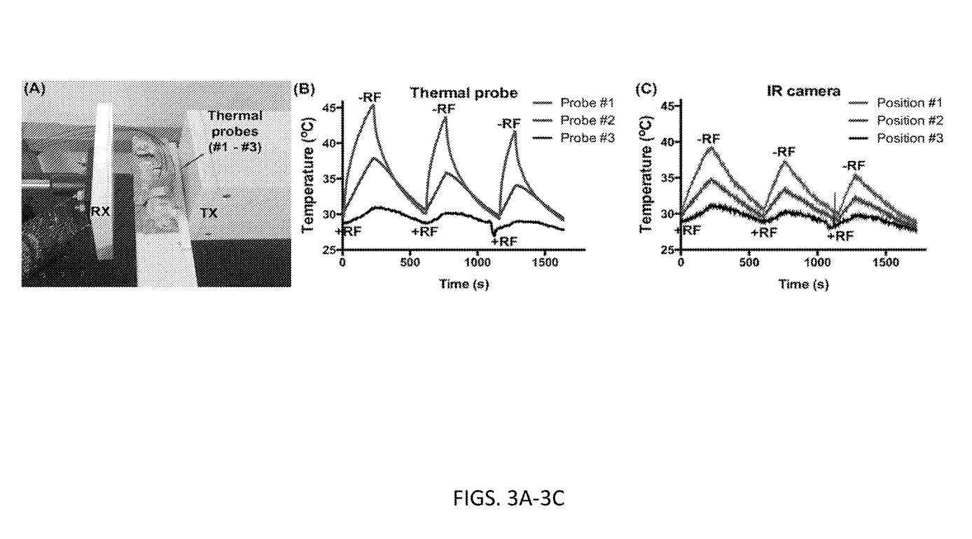

We initially exposed a 4T1 tumor-bearing mouse to the p-RF system to verify RF-induced biological heating. FIG. 3A depicts the experimental setup. The mouse was placed on a specially designed Teflon stage covered with a thin film of copper tape to electrically ground the mice: preventing surface electrical charge accumulation that would cause thermal injury. This stage is then inserted between the active TX-RX heads of the p-RF system. Three fiber optic thermal probes were directly inserted into the mouse at different positions around the tumor and distances from the TX head. Probe #1 (closest to the TX head) was inserted under the skin but above the tumor mass; probe #2 was inserted under the skin in between the area where the tumor is projected from the main body of the mouse; and probe #3 was inserted under the skin above the intraperitoneal cavity. Given that probe #1 would likely heat the most due to its proximity to the TX head, we used this as a reference in turning on and off the RF system at different temperature points: 45° C., 43° C. and 41° C., allowing the mouse to cool down to around ˜30° C. between each RF exposure. The total power needed to generate these heating profiles was only 90 W. An increase in power would result in significant thermal injury to the mouse due to rapid heat production. As can be seen in FIG. 3B the tumor temperature first increased from 30° C. to 45° C. in ˜250 s, taking ˜375 s to cool back down to 30° C. At this point the RF was turned back on and the tumor allowed to heat up to 43° C. before being turned off again. This was further repeated for a final tumor temperature of 41° C. The temperature data from probes #1-#3 clearly demonstrates the reduction in tissue heating due to the fall off in electric-field intensity from the TX head. If the electric field were to be constant across the TX and RX heads, such as that approaching the condition of an ideal parallel plate capacitor model, then any fluctuations and variations in temperature would be the result of the differences in permittivity and conductivity between the tissues, organs, and tumors of the mouse.

IVM-RF System

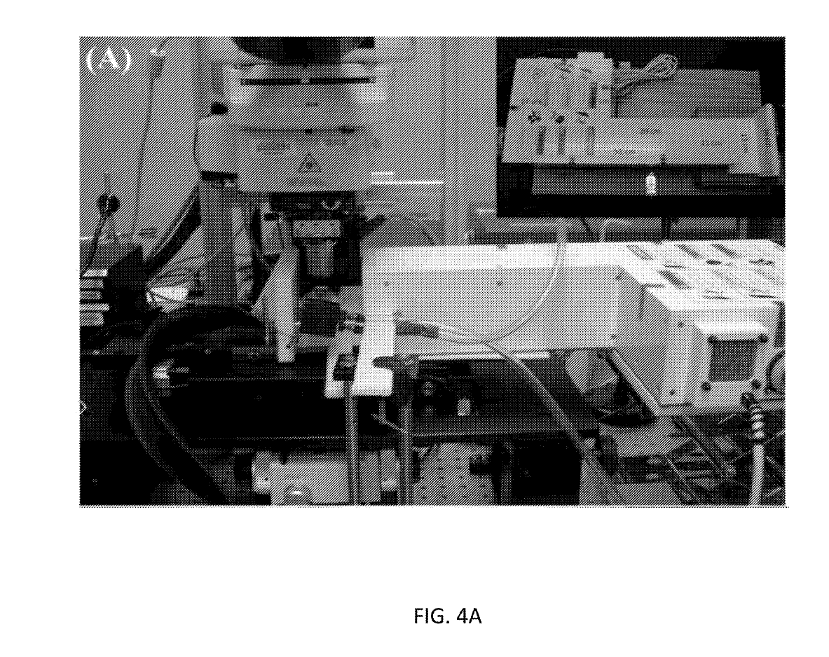

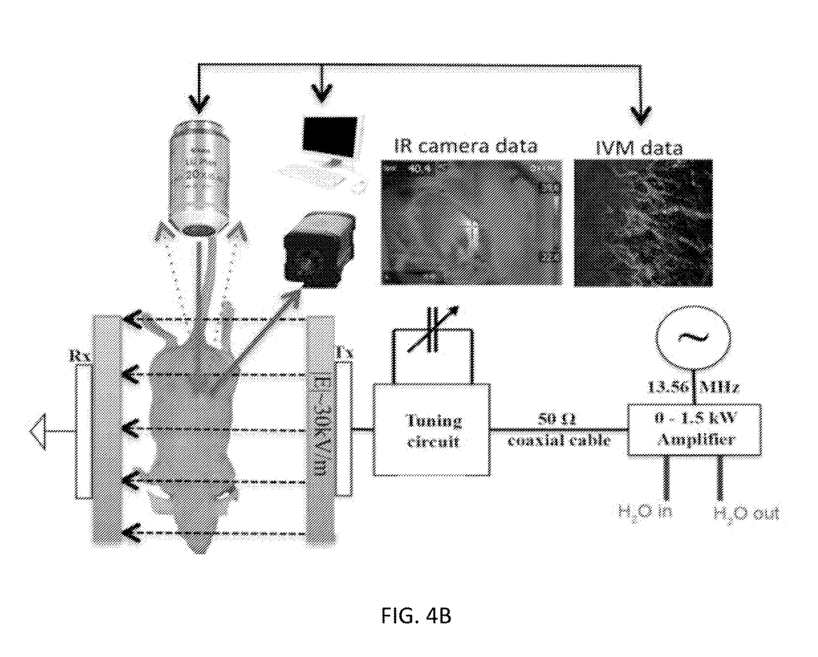

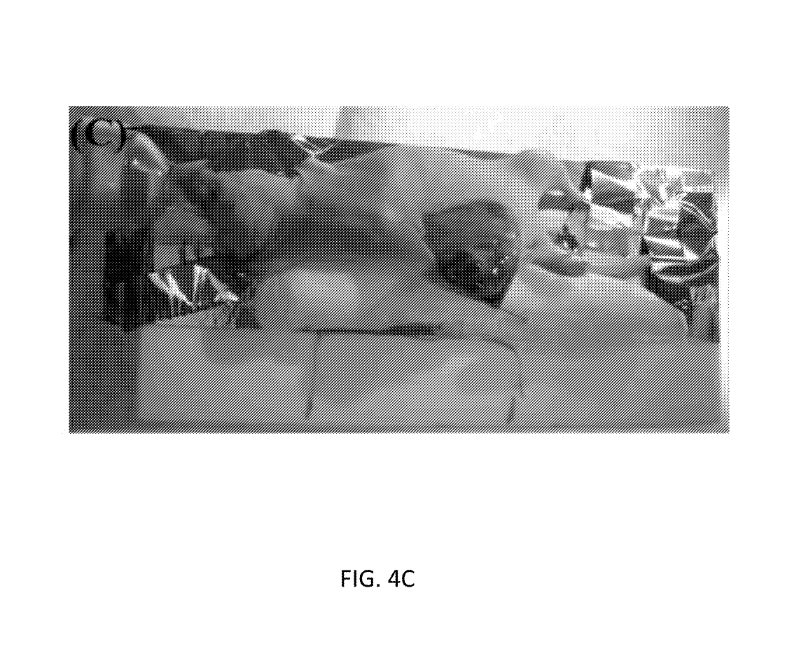

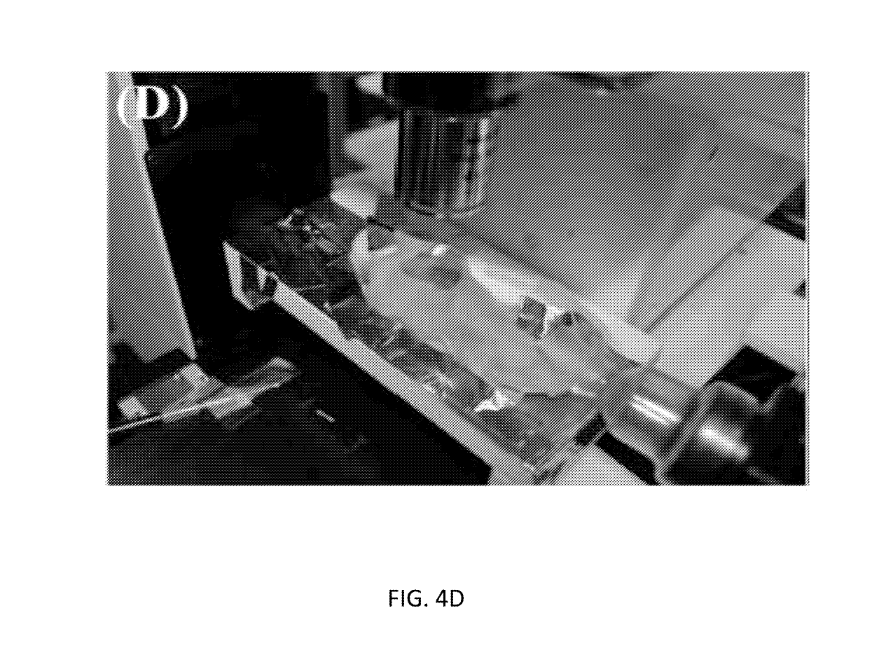

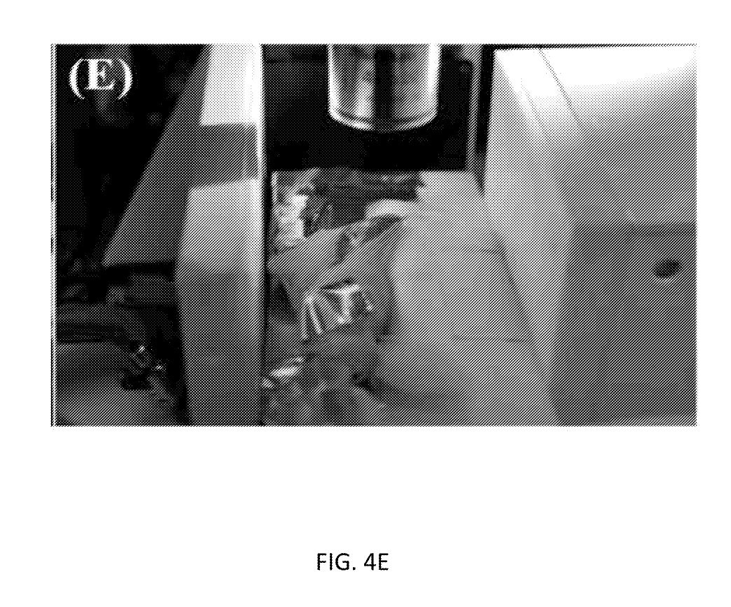

A picture of the p-RF system retrofitted to a Nikon AIR IVM is shown in FIG. (4A). The Nikon AIR laser scanning confocal microscope is equipped with a resonance scanner, CCD color camera (Nikon DS-F1), motorized stage (Prior Scientific ZDeck), Nikon long-working distance air plan-apochromat objective (4×, NA 0.2, WD 2 mm), and image acquisition software (Nikon NIS Elements 4.0). Once fitted to the IVM, our initial tests involved gradually increasing the p-RF power (without a sample) whilst monitoring the voltage induced across the IVM chassis by connecting an oscilloscope probe to the electrode ground pins located behind the objective lens on the IVM system. Even at 200 W RF power the voltage induced on the chassis was <500 mV, which is deemed negligible and would not interfere with hardware. This test procedure was performed to make sure the RF energy was not directly coupling to the IVM microscope, which would most likely cause irreversible electronic and structural damage to the IVM system. The only interference noticed was that several software programs and browser windows would randomly open and text would be randomly written in text fields—we termed this effect ‘ghost writer’ and discovered the origin of this effect to be due to RF fields coupling onto the computer keyboard. Wrapping the keyboard cable around a ferrite core balun to reduce RF interference solved this problem. A schematical representation of the full imaging platform is given in FIG. 4B. In brief, mice were placed on a specially designed Teflon stage. This stage is then inserted between the active electrodes of the RF system (i.e. the TX and RX heads) and positioned so that the exposed tumor can be imaged via intravital micrsocopy (IVM) whilst under RF exposure (FIGS. 4C-4E). An infrared (IR) video camera (FLIR SC 6000, FLIR Systems, Inc., Boston, Mass.) was also setup to allow surface temperatures to be monitored.

RF-IVM animal manipulation. Mice 4T1 tumors were exposed by a small midline incision whereby the fascia between the skin and muscle was disrupted using a cotton swab. An inverted skin flap was elevated using rolled cotton gauze. Images of the mice being manipulated for RF-IVM are shown in FIGS. 4C to 4E. Mice were anesthetized using 3% isoflurane (Aerrane; Baxter Healthcare) administered through an isoflurane vaporizer system (E-Z Systems). Mice were kept on a warming pad prior to experiments to maintain core body temperature. The tumor was moistened with saline and scanned at a video-rate of 30 fps Field of views were imaged using a frame rate of 30 fps at 250×250 μm every 1-5 minutes.

Fluorescent markers. The fluorescent markers used in these experiments were Albumin-Alexa Fluor 647 (MW˜66 kDa) and fluorescein isothiocyanate-dextran (FITC-dextran, MW˜70 kDa). Both were obtained from Life Technologies, Grand Island, N.Y. When used, mice were given 50 μl retro-orbital injections of either Alexa 647 or FITC (or both) at concentrations of 10 mg/Kg (suspended in phosphate buffered saline, PBS). The mice were then subjected to RF exposure with or without simultaneous IVM imaging. Fluorescent tracers were used in this study to stain the tumor blood vessels as and to investigate the enhanced uptake and perfusion of tracers into tumors, which are fluorescent due to their transfection with a tdTomato-fluorescent protein). The 4T1 tdTomato-expressing tumor was excited at 561 nm and data was collected using band-pass filter widths of 30-50 nm centered at 647 nm for albumin and 579 nm for tdTomato. 512×256 bit two-channel images were acquired with a pinhole of 1.0 Airy unit. FITC-dextrans were imaged using an excitation wavelength of 488 nm with band-pass filters widths of 30-50 nm centered at 520 nm. After each imaging experiment, animals were sacrificed and in some cases tumors excised and frozen in cryo-embedding media (OCT) for immunohistochemical analysis.

In the experiments where fluorescein isothiocyanate dextran (FITC-dextran) markers were used for mice bearing orthotopic PANC-1 pancreatic tumors, the tumor red channel was not imaged, as PANC-1 cells expressed no fluorescent proteins. FITC-dextran (Sigma Aldrich, St. Louis, Mo.) of size ˜70 kDa MW was administered by retro-orbital injection of 10 mg/kg in 50 μl PBS and imaged for 30 minutes, under RF, using an excitation wavelength of 488 nm with band-pass filters widths of 30-50 nm centered at 520 nm. Once the RF was turned off, albumin (Alexa Flour 647) was injected and imaged, with the FITC-dextran, for a further 30 minutes.et al.et al.i.e.

Immunofluorescent imaging. The complete macro-perfusion and uptake of fluorescent tracers throughout the tumor in RF and non-RF treated mice was also analyzed ex vivo using immunofluorescent imaging. This allowed for comparison to the analysis of perfusion of the tracers into the tumor at μm depths obtained using the IVM system. Also, by staining with antibodies for CD31, which allows for the imaging of intracellular endothelial cell junctions, we can verify that the albumin or FITC-dextran is perfusing out of these vessels. The frozen sections of the tumor tissue were fixed with 4% paraformaldehyde, blocked with 5% normal horse serum and 1% normal goat serum in PBS, and immunofluorescently stained using antibodies to CD31 (BD Biosciences, San Jose, Calif.). Sections were then incubated with goat anti-rat IgG Alexa Fluor 488 antibody (Jackson ImmunoResearch, West Groove, Pa.). The images were captured using Nikon Al confocal microscope and analyzed using Nikon Elements v3.2. The ratio of pixels in the whole image that has higher fluorescence intensity over the threshold (background) was shown as positive area fraction. The data were shown as the average±standard deviation from representative sections of more than 5 images of tumors.

Algorithms for quantifying fluorescent tracer perfusion. We have created algorithms to quantitatively monitor events such as fluorescent tracer tumor accumulation or extravasation from blood vessels. Using data acquired from live animals treated with albumin or FITC-dextran by IVM we first segment the tumor to generate a binary image from the fluorescent tumor image (i.e. pixel=0 if pixel intensity<threshold, pixel=1 if pixel intensity≧threshold). When we have the binary image we generate a mask, which represents the tumor (to do this we use simple dilate and erode operations to remove small holes and smooth edges). Next, we determine the overlap of the tracer fluorescence with the tumor mask to find pixels, which have tracer fluorescence in the tumor mask. Lastly, we find the tracer fluorescence that has leaked from the vasculature system and determine a mask for the vasculature systems using a binary threshold on either the FITC or albumin channel to calculate the tracer leakage, which is not in the vasculature mask and integrate the intensity over these pixels to get total intensity. The final step gives the amount of albumin or FITC-dextran, which has leaked from the vasculature system into the tumor.

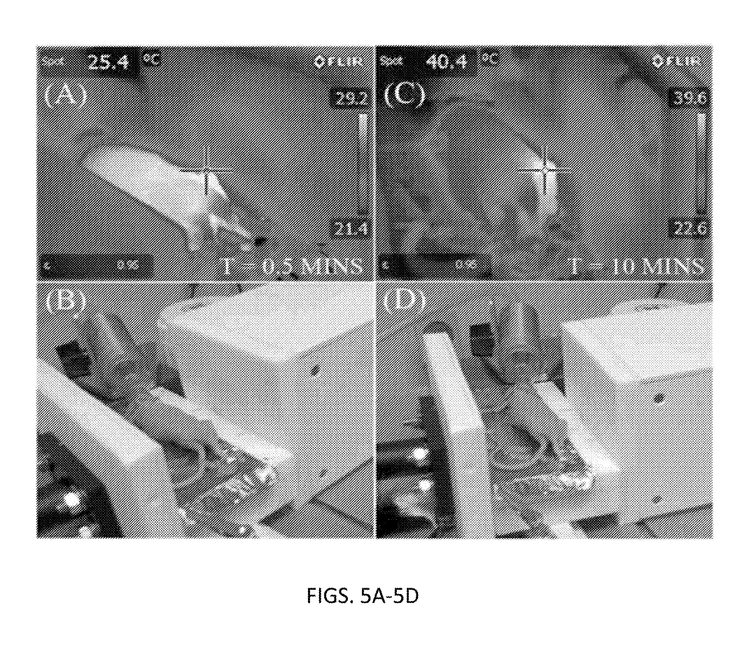

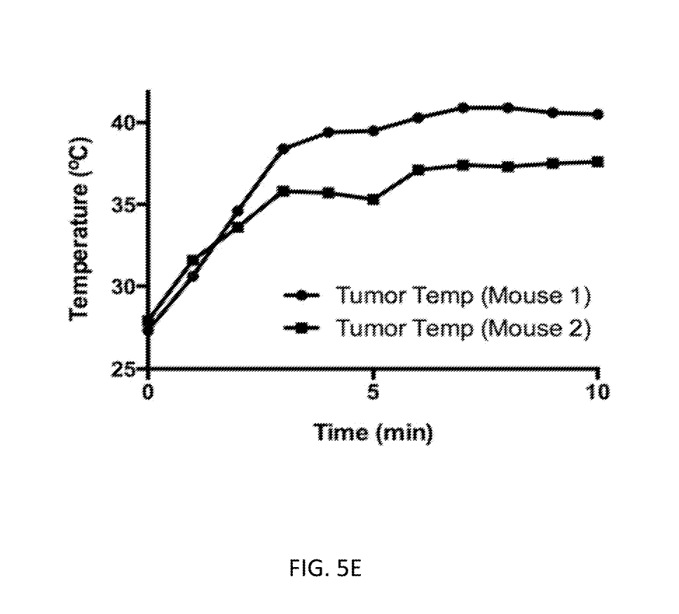

FIGS. 5A and 5C depict elevations in tumor temperature, taken using the IR camera, on mice subjected to RF exposure for a period of 10 minutes (without IVM). Shortly after initiating RF exposure (T=30 seconds), the tumor temperature reached 29.2° C., slightly higher than the average body surface temperature of ˜27° C. Within a couple of minutes of exposure the tumor surface temperature ranged from 37-41° C. and was maintained over the 10-minute exposure. Irreversible cellular injury occurs with focal heating at 50-60° C., which can occur in 4-6 minutes with conventional RFA, and causes coagulation and necrosis (see, e.g., Goldberg, et al., 1996). However mild temperature increases are tolerated by cellular homeostatic mechanisms, with temperatures between 39-45° C. causing reversible cellular injuries. The complete temperature versus time profile two mice, (exposed to the same conditions) is shown in FIG. 5E. As can be seen, these temperature ranges are well within the mild hyperthermia range indicating that irreversible cell damage should not have taken place. A visual inspection of the mouse after RF exposure revealed no surface burns or swelling, which would be indicative of thermal injury.

The electric field intensity around the tumor as well as the tumors' dielectric properties are perhaps the two most important physical parameters governing the heating rates of individual tumors. Dielectric in this case refers to how much electrical energy a material will absorb and convert to heat, and is frequency dependent. A recent publication has showed anti-tumor effects using just non-invasive RF. In their study, Raoof et al. subjected mice bearing orthotopic-implanted human hepatocellular and pancreatic xenografts to weekly RF exposures. Their results indicated that RF-alone was enough to cause an anti-tumor effect in hepatocellular carcinomas and could be explained purely on the principle of the tumors' dielectric properties being larger than normal, healthy tissues. The ability of a material to store and dissipate electrical energy as heat can be described by the real (ε′) and imaginary (ε″) parts of the complex permittivity function (ε*). This relationship is given by equation 1:

ε*(ω)=ε′(ω)−iε″(ω) (1)

where ω is the radial frequency (2πf). The real term of equation 1 gives information as to how much electrical energy can be stored in a material whilst the imaginary term denotes how much of this energy is converted to heat.

In a purely ideal situation, the imaginary values for tumor tissues would be significantly higher than that of normal, healthy tissues, whereby the tumor would heat rapidly up to temperatures that induce either hyperthermia (leading to natural programmed cell death mechanisms) or complete ablation and necrosis. The dielectric properties of both cancerous and normal tissues were measured by Raoof et al. (using a permittivity analyzer), and were shown to be larger for tumors than normal cells.

The relationship between a materials permittivity and its effect in heat production when exposed to a time varying electric-field is given by the following equation:

Heating Rate (HR)=dT/dt=(ε⁰ ε″(ω) |E|²)/(2ρ*Cₚ) (2)

where ε₀ is the vacuum permittivity, ε″ is the imaginary part of the complex permittivity, E is the electric field intensity in the sample, ρ is the density, and cₚ the specific heat capacity. In this fundamental governing equation, all of the relevant physical variables are contained that describe how a sample will respond to exposure to an electric field. This equation, especially the strong dependency on electric field intensity, can help further explain the decrease in heat production as the temperature probes are located further away from the TX head due to the gradual reduction in electric-field strength

Multi-Channel IVM-RF Imaging and High-Temperature Vessel Degradation

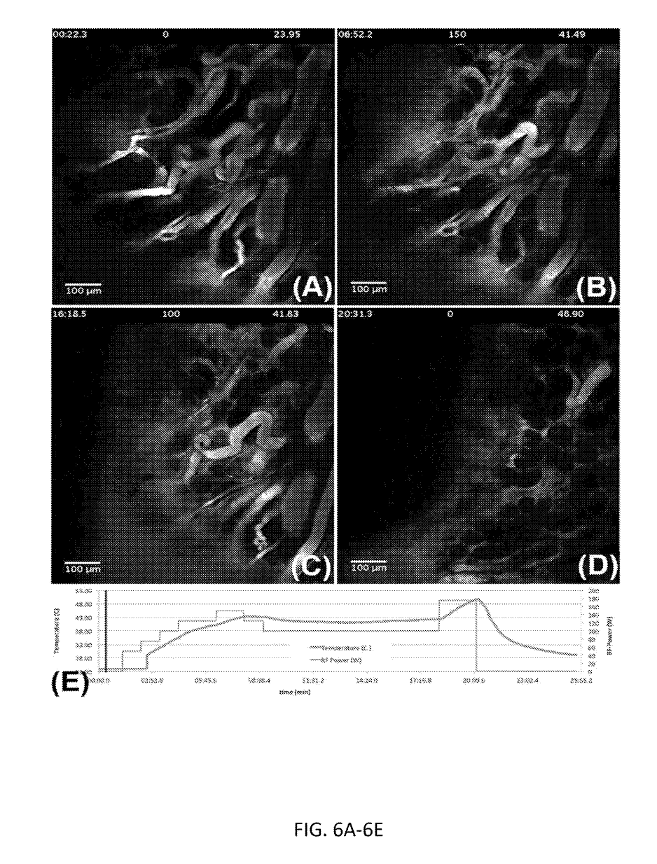

The effect of vessel degradation after prolonged periods and elevated temperatures of non-invasive RF exposure (i.e.>45° C.) can be seen in FIG. 6. By looking at the four different time points (FIGS. 6A-6D) it can be seen that some low level of vessel degradation is evident for temperatures between 41.5-41.8° C. (we were able to hold this temperature range for ˜10 minutes). After this, upon application of more RF power the increase in tumor heat production (up to ˜49° C.) resulted in severe degradation and complete shutdown of the tumor vessels.

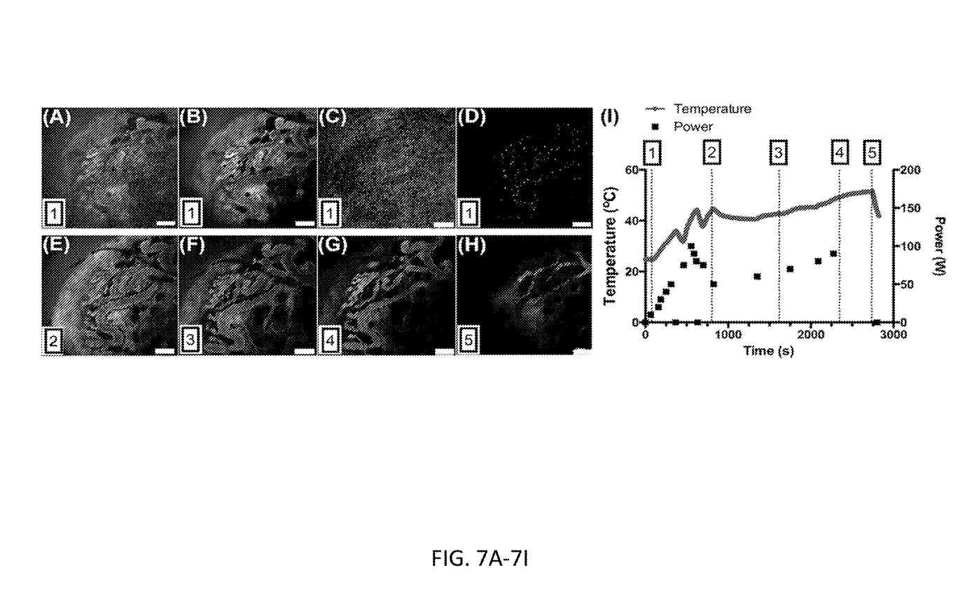

FIG. 7 depicts real-time multi-channel IVM-RF imaging on an exposed 4T1 breast tumor. Three separate channels were imaged: FITC (vessels), Texas Red (4T1 transfected tumor), and Cy5 (red blood cells, RBCs). FIG. 7A shows the individual channels merged into one image, whilst the individual channels are shown in FIGS. 7B-7D. FIGS. 7E-7H shown changes in the vessel architecture for four different time points—illustrated as time points numbers 1 through 5 in FIG. 7I (NB: time point number 1 corresponds to imaging before the addition of RF exposure). Also shown in FIG. 7I is the graph of tumor temperature and RF power versus time. Tumor temperature in this case was monitored using a temperature probe placed in the tumor.

As can be seen from these results, the tumor vessels start to narrow and constrict once the tumor temperature is raised above 41° C. For a final tumor temperature of 44° C., the vessels have completely coagulated and stopped functioning. Once the temperature is elevated above 41° C. the flow of RBCs becomes irregular and there are some vessel compartment where blood flow has ceased completely. Note, with regards to the time points where the RF power was intermittently stopped (i.e, 0 W) this was initiated to stop overheating and buring of the tumor sample from rapid heating. We have found from previous experiments that tumors will burn of they are heated too quickly. The use of a rapid heating profile also allows us to tailor off the power once the designated temperature is reached so that an accurate set temperature can be maintained. This can be seen from time points 2 and 3 where the power is rapidly decreased then gradually increased to allow for a more gentle heating profile.et al.et al.et al.et al.

RF-Induced Fluorescent Tracer Transport and Perfusion into 4T1 Tumors

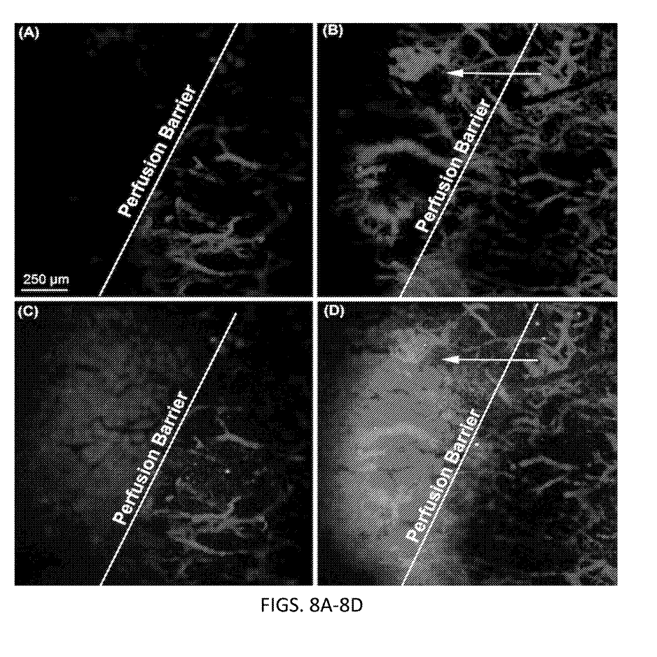

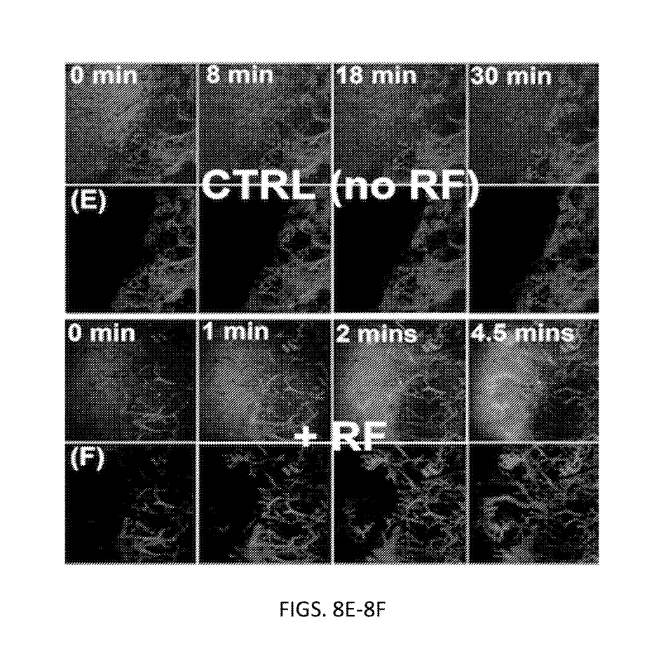

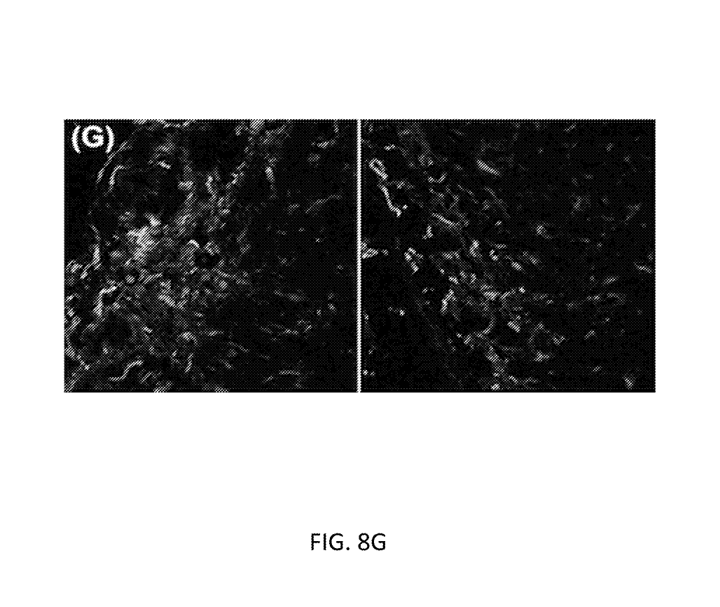

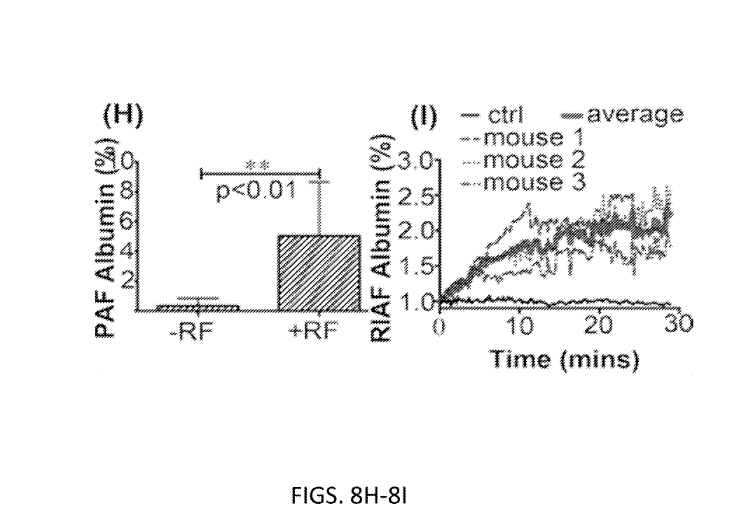

Mice with 4T1 tumors were prepared for RF-IVM as mentioned in the methods section. In our first tests, we injected 50 μl of the albumin-alexa-fluor 647 dye (10 mg/Kg) into mice via retro-orbital injection and imaged with and without RF (as a control). For all experiments the RF was turned off once the tumor temperature reached 41° C. (unless otherwise stated) as indicated by the IR camera. FIGS. 8A-8D depicts perfusion of the albumin tracer out of blood vessels and into the tumor for an RF treatment duration of 4.5 minutes. This is particularly evident when comparing FIGS. 8A and 8B for the start and end-point (0 and 4.5 minutes, respectively) for the albumin only (blue) channel. For a control, the same experiment was performed without RF exposure (FIG. 8E) for 30 minutes. An impaired perfusion barrier is evident as no albumin was able to penetrate and be transported into the tumor. Impaired perfusion reduces oxygen supply, causing a hypoxic microenvironment, and decreases systemic drug delivery. This is often caused by factors such as leaky vasculature walls and increased tumor blood vessel compression. As can be seen in FIG. 8E even after 30 minutes very little albumin perfused from the blood network into the tumor. This was repeated several times on different mice for durations up to 60 mins whereby the same limited perfusion phenomenon was observed.

Post RF Analysis

Immunofluorescent staining was performed on RF and non-RF treated tumors (n=6), as described in the methods section, to visualize the distribution of the albumin tracer from the vessels. This can be seen in FIG. 8G. Note, these tumors were resected from the mouse and cut in half to expose the tumor. The evidence of albumin (seen as red in the figure) in close proximity to the vessel endothelial cells (seen as green in the figure) is only evident for the tumors treated with RF (left-hand image in FIG. 8G). The ratio of pixels in the whole image that has higher albumin fluorescence intensity over the threshold (background) was shown as a positive area fraction (PAF) graph, which can be seen in FIG. 8H and indicates five times enhancement of albumin perfusion into the tumor for RF-treated mice when compared to the controls. The use of an imaging algorithm (methods section) to quantitate the surface perfusion of fluorescent tracers into tumor tissue directly from the real-time IVM imaging data was also utilized (FIG. 8I) to depict on average a doubling of surface perfusion of the fluorescent tracers in the tumors when compared to the controls.

Prolonged RF-Induced Biological Effects

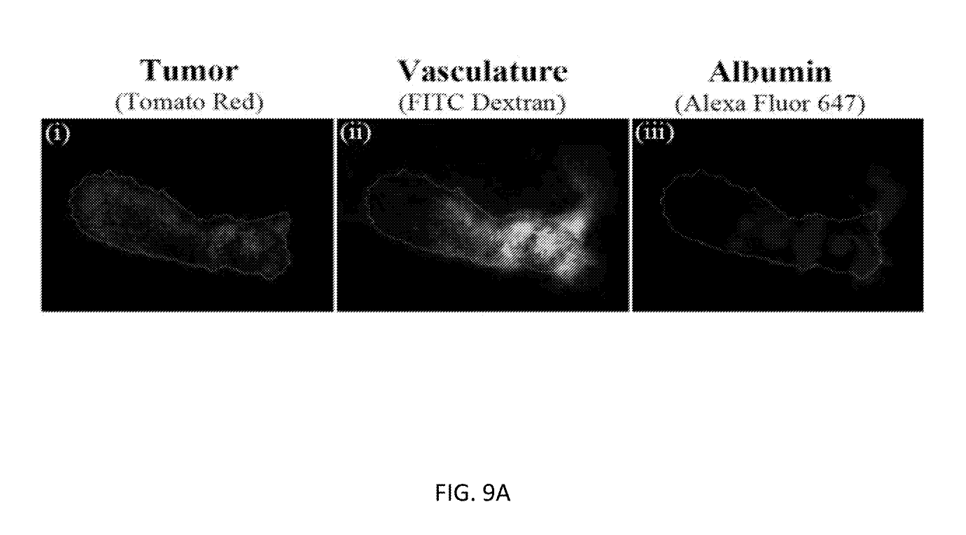

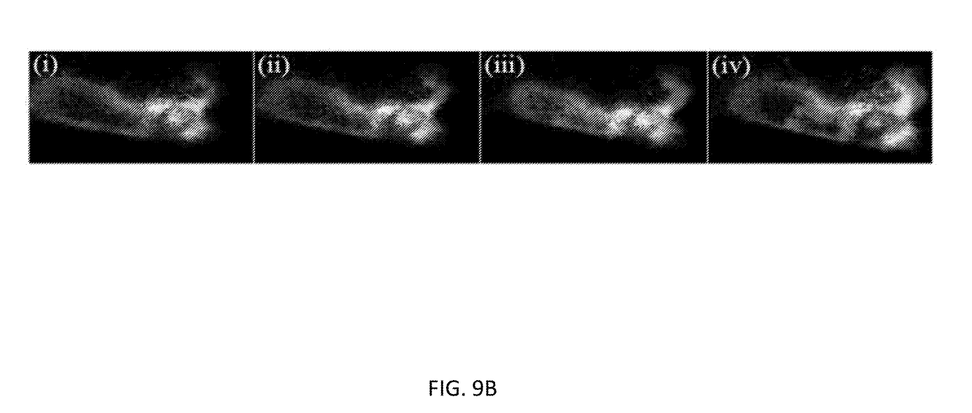

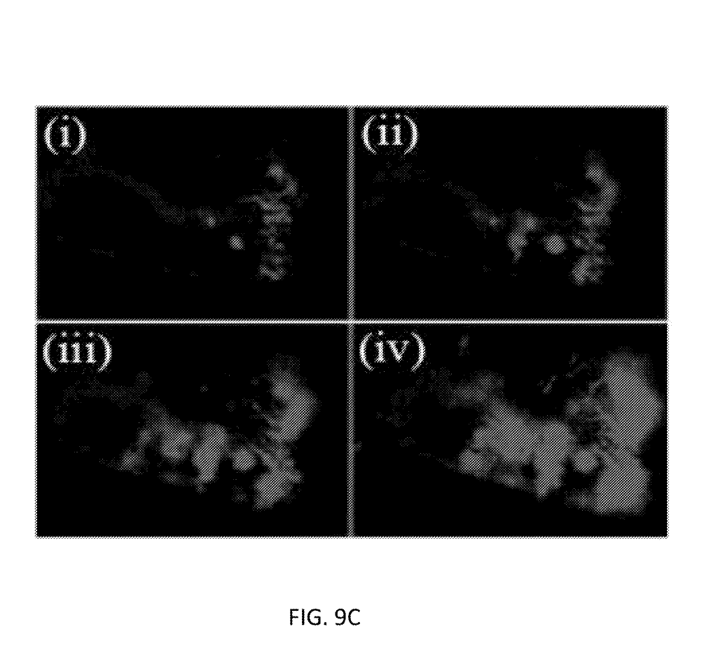

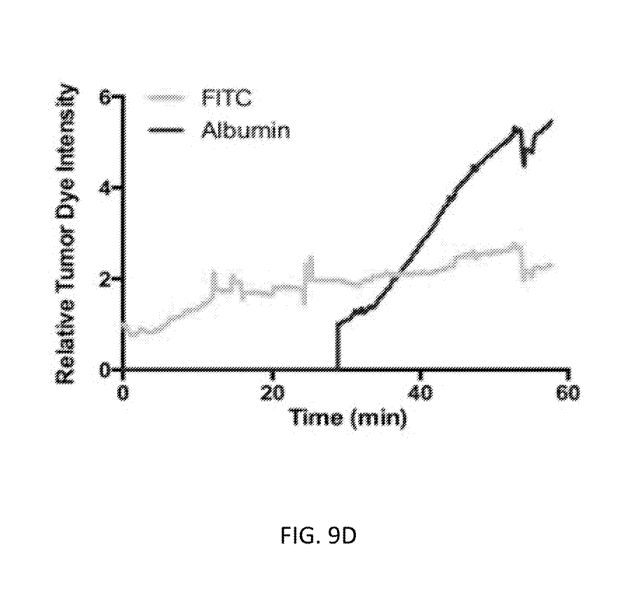

In order to see if the tumor perfusion of fluorescent tracers is evident after RF exposure we performed further experiments whereby fluorescent tracers were administered after RF exposure. Mice bearing 4T1 tumors were administered 50 μl FITC-dextran (10 mg/Kg) via retro-orbital injection, and exposed to RF for 30 minutes. After RF exposure, we then administered 50 μl albumin (10 mg/Kg) and performed IVM imaging for another 25 minutes, without RF exposure. These results (as well as the technique used for analysis) are shown in FIG. 9. FITC-dextran was injected followed by 30 mins RF exposure. In FIG. 9A The tumor area (i) is demarcated using a green line and allows FITC-dextran (ii) and albumin (iii) perfusion to be monitored. In FIG. 9B these masks are applied to the full time-lapsed video for all channels. In FIG. 9C areas where both albumin and FITC-dextran overlap are processed to quantify the relative average intensity of albumin perfusion after 30 mins of RF exposure. FIG. 9D shows the relative tumor dye intensity (RTDI) versus time. The relative tumor dye intensity (RTDI) increase in FITC (as shown in FIG. 9D) is shown to gradually increase over time and continues after RF exposure. This is also true for albumin, which shows a rapid increase in RTDI once administered immediately after RF exposure. This suggests the effects of RF energy on the tumor vasculature system enabling increased perfusion of fluorescent tracers into the tumor are long-lived, at least for up to 25 minutes after RF exposure. Given that both tracers are of similar molecular weight (albumin=66 kDa, FITC-dextran=70 kDa) it is interesting to see an increase in perfusion of albumin over the FITC-dextran.

RF-Enhanced Perfusion in Orthotopic Panc-1 Pancreatic Tumors

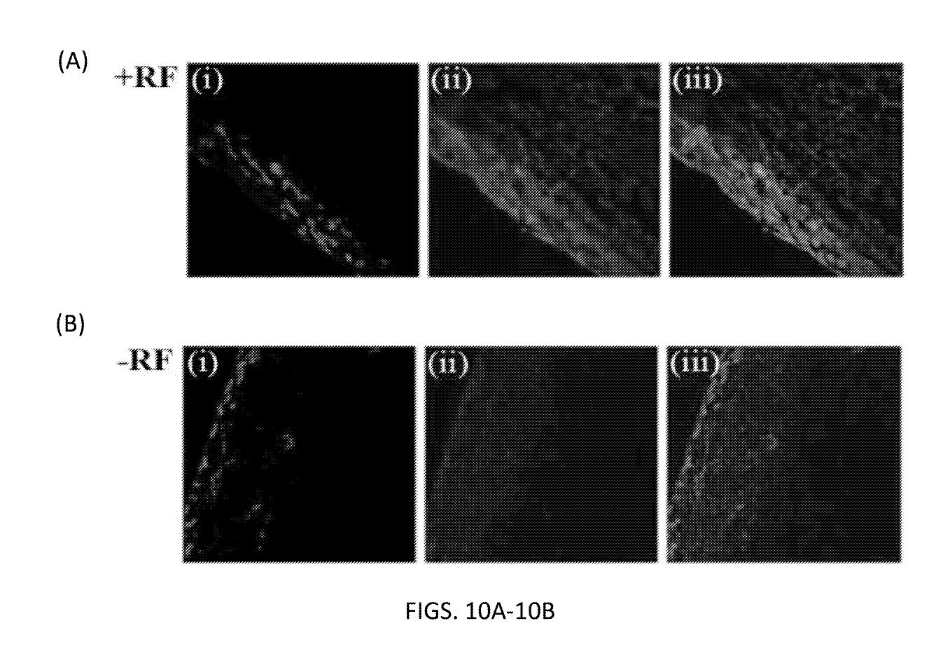

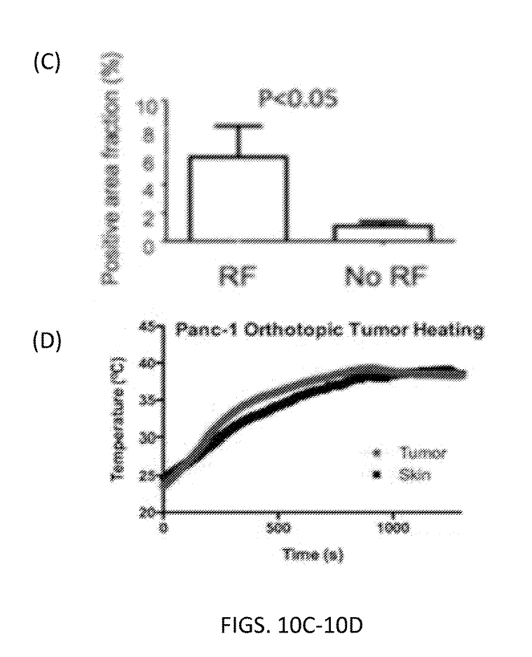

Similar experiments were performed on mice bearing orthotopic PANC-1 pancreatic tumors but with the use of albumin alone. In this instance, fiber-optic probes were inserted directly into the tumor (as well as the skin in the immediate vicinity) to take real-time temperature measurements. As can be seen in FIG. 10, similar to the immunofluorescently stained post-RF samples there is evidence of enhanced albumin perfusion out of the vasculature endothelial vessels into the surrounding tumor tissue. FIG. 10C indicates that there is approximately six times more albumin in the pancreatic tumor with RF when compared to the control.

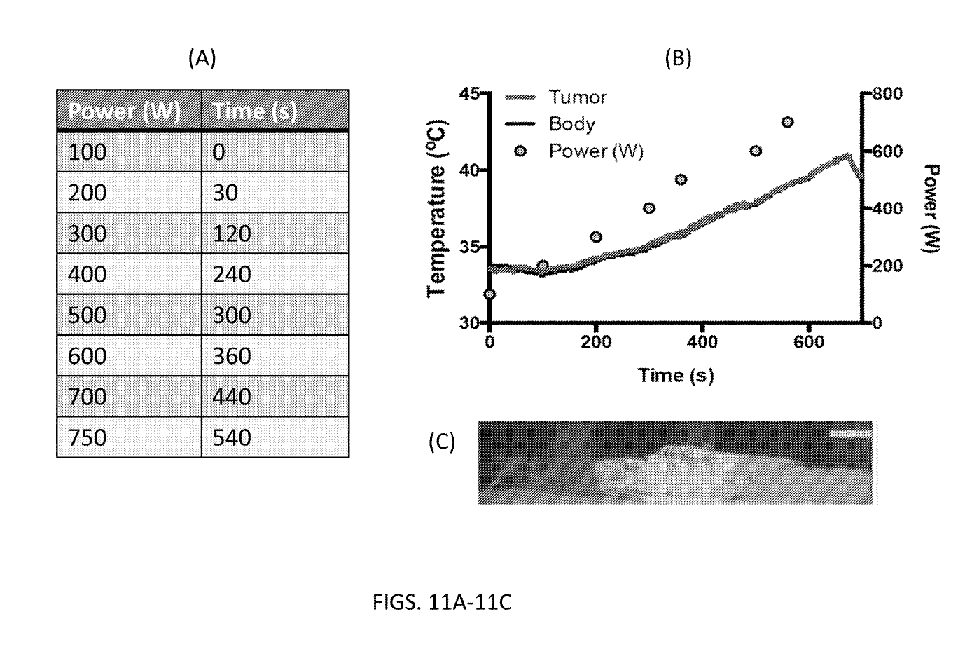

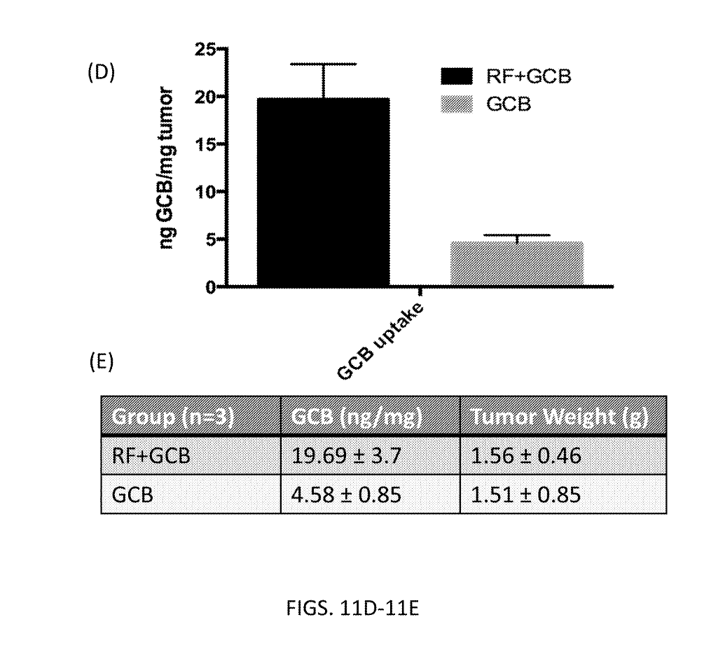

FIGS. 11A-11E illustrate an example of an embodiment of the use RF energy to enhance the uptake of chemotherapy drugs into tumors compared to using chemotherapy drugs alone. (FIG. 11A) Female Nude mice (4-6 weeks old) were given direct injections of KPC pancreatic adenocarcinoma cells (1×10⁶) into the pancreas. After 1-2 weeks (enough time for a orthotopic pancreatic tumor to form) the mice were subjected to RF exposure under a ramped power treatment protocol as shown in the table. (FIG. 11B) Graph of a typical RF power and resulting temperature versus time plot for a single mouse being exposed to RF energy. The surface temperature of the mouse in close proximity to the pancreas was recorded using an IR camera. The RF exposure was turned off once the temperature reached 41° C. (FIG. 11C) IR camera image of the mouse at the end of the treatment. (FIG. 11D) Example of enhanced uptake of Gemcitabine. Six mice with orthotopic pancreatic tumors were given systemic injections of Gemcitabine at a dosage of 70 mg/Kg. Three of the mice were immediately exposed to RF conditions shown in (FIG. 11B) and were then sacrificed 5 hours later and compared to the non-RF treated mice, also sacrificed 5 hours after administration of Gemcitabine. There is approximately 4× more Gemcitabine present in the tumors of the RF-treated mice than the control group indicating non-invasive RF exposure enhances uptake of chemotherapeutics into tumors. Quantification of Gemcitabine was analyzed using Liquid Chromatography Mass Spectroscopy (LC-MS). FIG. 11E shows quantification of the levels of Gemcitabine in the orthotopic pancreatic tumors of mice with and without RF exposure. Total tumor weights are also shown and are approximately equal in size/mass.

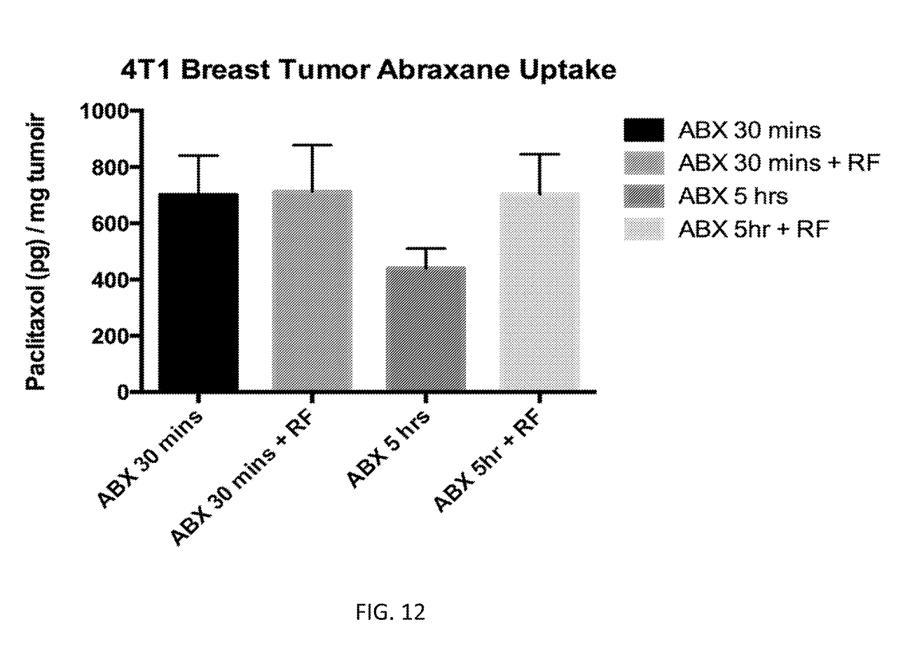

FIG. 12 illustrates an example of an embodiment of the use non-invasive RF energy to enhance the uptake of chemotherapy drugs into tumors compared to using chemotherapy drugs alone. Abraxane intravenous injections were given to nude mice bearing orthotopic 4T1 breast tumors. Dosage was 125 mg/kg. Tumors were harvested at 30 mins and 5 hrs after injection, with and without RF exposure (conditions similar to that described in FIG. 11 B). The active ingredient in abraxane, Paclitaxol, was analyzed using LC-MS. As can be seen, there is clear evidence of enhanced retention of paclitaxel in the tumors even for 5 hours post-RF exposure. The enhanced retention observed at 5 hours may be due to high-temperature vessel degradation and coagulation, which would lock the drugs inside the tumor preventing them from leaving the tumor micro-environment.

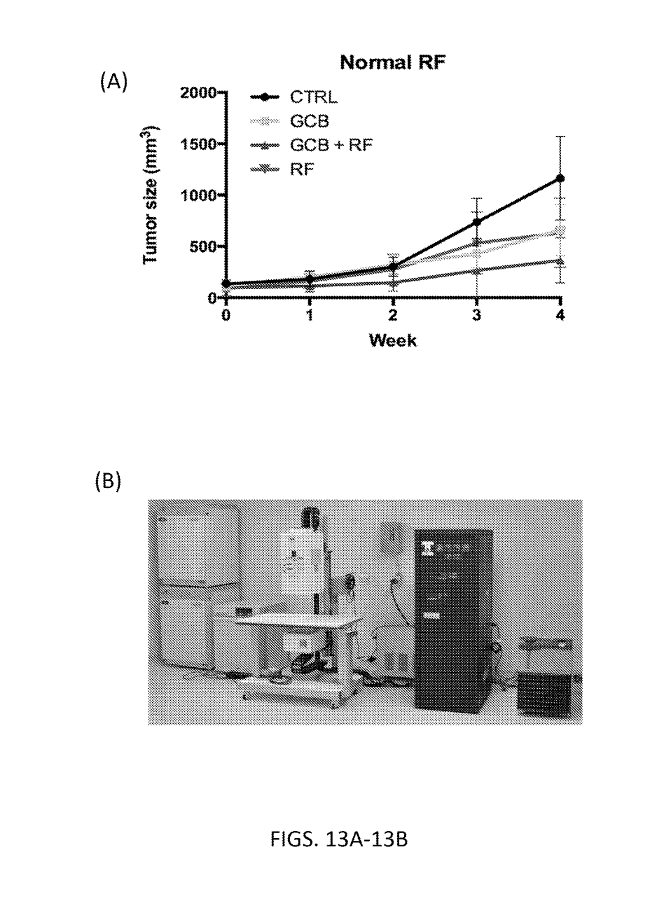

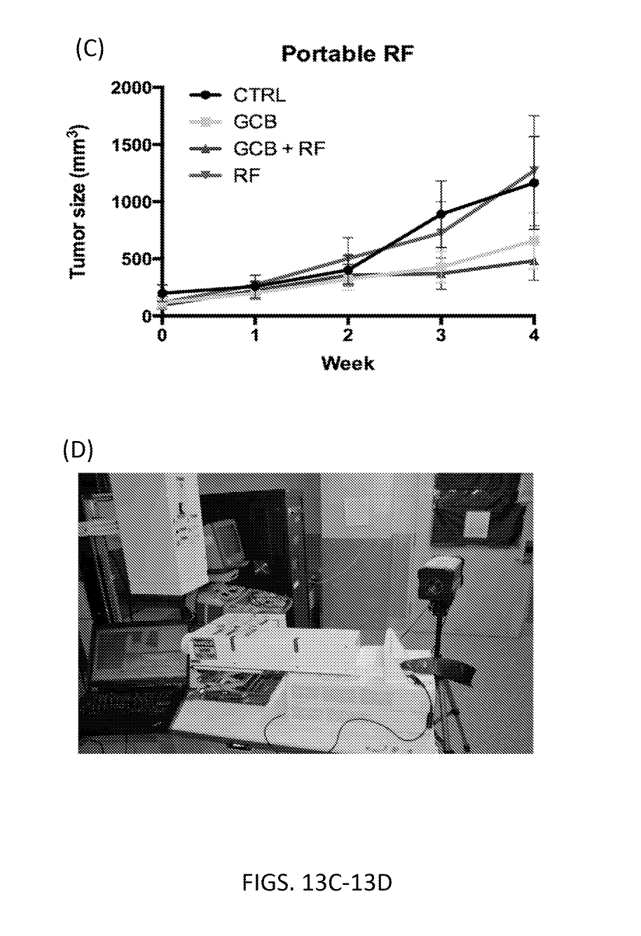

FIGS. 13A-13D illustrate an example of an embodiment of the use RF energy to provide synergy between RF exposure and chemotherapeutics such as Gemcitabine. (13A) Mice bearing subcutaneous pancreatic tumors were exposed to non-invasive RF, Gemcitabine, or non-invasive RF+Gemcitabine once per week until the control group tumor size became a burden (n=5 in each group). The non-invasive RF system used can be seen in (13B) and is a higher power version (0-1200 W) of the portable RF system. non-invasive RF conditions were similar to that shown in FIG. 13B. Gemcitabine was given intravenously at a dosage of 70 mg/Kg. As can be seen in (13A) there is a clear reduction in tumor size in the Gemcitabine+non-invasive RF group when compared to the other groups indicating the synergistic effects of non-invasive RF on the efficacy of chemotherapeutics. (13C) The experiment was repeated using a portable RF probe system (smaller power, 200 W), which is shown in (13D). As can be seen a similar synergy effect between Gemcitabine and non-invasive RF is also evident even when using a lower power system. The mice in the portable non-invasive RF system experiment were exposed to 200 W of RF power for 15 minutes.

Kruskal et al. reported that RFA with lethal cell thermal doses results in sub-lethal hyperthermia in the periablational rim, characterized by increased vascular permeability and endothelial gap junction leakiness. Considering we kept all tumor temperatures within this sub-lethal 39-41° C. temperature range, this could possibly explain our enhanced albumin and FITC-dextran tumor perfusion observations. Monsky et al. also reported that conventional RFA leads to a seven-fold increase in the accumulation and retention of doxorubicin liposomes, but not free doxorubicin in the tumor. They proposed that this resulted, again, from increased vascular permeability attributed to sub lethal hyperthermia. The increased permeability is in addition to what already exists in the compromised leaky tumor vasculature, inducing a state of ‘hyperpermeability’.

Significance of Certain Embodiments

A custom portable RF system is described that can easily be retrofitted to high-resolution imaging modalities such as intravital microscopy (RF-IVM). This system allows for real-time image acquisition of RF-induced changes in vascular permeability; tissue integrity; and enhanced tumor distribution of blood-circulating fluorescent tracers such as albumin and FITC-dextran. In controlled in vivo tests on mice bearing orthotopic breast 4T1 and pancreatic PANC-1 tumors, short durations of RF exposure enhance perfusion and delivery of these macromolecules into and out of the tumor vasculature system. Post-RF image analysis using algorithms (applied to the captured IVM data) and immunofluorescently staining showed, on average, a two to six-fold increase in albumin and/or FITC dextran delivery into the tumor. The results demonstrate the use of the RF-IVM system as a powerful visualization tool in assessing, optimizing, and exploring the basic science behind non-invasive RF cancer therapy. Also shown in some examples are the quantification of the uptake of chemotherapeutic drugs (examples shown are for gemcitabine and abraxane) into tumors through the use of exposure to non-invasive radiofrequency hyperthermia.

### Example 2

### Hyperthermia Inhibits Recombination Repair of Gemcitabine-Stalled Replication Forks

Primary hepatocellular carcinoma (HCC) is an aggressive disease. Globally, about one million new cases of HCC are diagnosed each year with an identical cause-specific mortality rate (Vautthey, et al., 1995). Most patients are not eligible for curative intent local-regional therapies (Vautthey, et al., 1995). Conventional cytotoxic therapy has been shown to be of minimal benefit. This has largely been attributed to over-expression of multidrug resistance-associated efflux protein (Soini, et al., 1996; Huang, et al., 1992; Caruso, et al., 1999). The clinical response of HCC to sorafenib, a multi-kinase inhibitor, is limited and transient (Llovet, et al., 2008). The investigation was focused on gemcitabine, which is not used for the treatment of HCC because of substantial chemoresistance.

Gemcitabine (2′,2′-difluoro 2′-deoxycytidine, dFdC) is a nucleoside analogue and a prodrug that is incorporated into the DNA of replicating cancer cells after activation. Despite resistance of HCC to gemcitabine, the mechanisms of resistance are largely elusive. Study of gemcitabine resistance in other cancers has focused on pathways involving its transport and metabolism, or those of altered apoptosis and survival. It is unclear if mechanisms engaged in repairing gemcitabine-stalled replication forks are important in resistance. Aberrant mismatched nucleotides are removed from the DNA by 3′-5′ exonuclease activity of DNA polymerase ε (Huang, et al., 1991). It was demonstrated that dFdCMP residues are difficult to excise from the DNA, in part because of masked-chain termination in comparison with dCMP residues (Gandhi, et al., 1996). Recent data suggests that restart requires regression of the stalled fork into a chicken-foot structure (Helleday, et al., 2003). This replication fork intermediate is sensed by poly (ADP-ribose) polymerase 1 (PARP1). Poly-ADP ribose residues associate with the chromatin recruit Mre11, a 3′-5′ exonuclease for DNA end processing (Bryant, et al., 2009). The DNA end processing is essential for loading of Rad51 recombinase on the DNA that forms a RAD51 nucleoprotein filament assisted by BRCA-2 (Saintigny, et al., 2001). This complex subsequently catalyzes sister chromatid homology search and strand invasion to complete homologous recombination repair (HRR) (Helleday, et al., 2003). The HRR pathway attempts to restore error-free replication; however, once malignant transformation has occurred, cancer cells may rely on DNA repair pathways such as HRR to propagate the mutated genome. Importantly, it has been clearly demonstrated that cells treated with replication inhibitors exhibit pronounced activation of HRR and that this pathway is essential for survival during recovery from stalled replication forks (Saintigny, et al., 2001). At least one report suggests localization of HRR pathway proteins to sites of gemcitabine-stalled replication forks (Wald, et al., 2008).

Recent studies have demonstrated that hyperthermia has pronounced inhibitory effects on HRR pathways, mainly mediated through inhibition of PARP-1, MRN-complex, or BRCA-2 in the context of DNA double strand break repair (Zaalishvili, et al., 2012; Martin, et al., 2009; Zhu, et al., 2001; Seno, et al., 2004; Gerashchenko, et al., 2010; Dynlacht, et al., 2011; Krawcyzk, et al., 2011). It is not known, however, if HRR of stalled replication forks is an important mechanism that contributes to chemoresistance of gemcitabine. In certain embodiments of the disclosure, hyperthermia can inhibit homologous recombination after gemcitabine-stalled replication forks through its effects on key components of the HRR pathway, hence contributing to chemosensitivity in hepatocellular cancer, a malignancy not usually treated with gemcitabine because of drug resistance.

Cell Lines, Reagents, and Transfection

All cell lines (Hep3B, HepG2, and SNU449) were purchased from the American Type Culture Collection (ATCC, Manassas, Va.) and used according to the supplier's protocol within six months of acquisition. For transfected cell lines, short tandem repeat fingerprint was confirmed by the Cell Line Characterization Core Service (M. D. Anderson Cancer Center, Houston, Tex.). Media, i.e., RPMI-1640 (for SNU449) or MEM (for HepG2 or Hep3B) was supplemented with 10% (v/v) fetal bovine serum. For fluorescence microscopy, the following primary antibodies were used: rabbit anti-PAR (Trevigen, Gaithersburg, Md.), rat anti-RPA32 (4E4, Cell Signaling, Danvers, Mass.), rabbit anti-Mre11 (GenTex, San Antonio, Tex.), rabbit anti-rad51 (H-92, Santa Cruz Biotechnology, Santa Cruz, Calif.), mouse anti-γH2AX (Upstate-Millipore, Billerica, Mass.), and rat anti-BrdU (BU^{1/75}[ICR1], Abcam, Cambridge, Mass.). Primary antibodies were detected using the following secondary antibodies; Alex Fluor 488 conjugated donkey anti-rat, Alex Fluor 546 conjugated donkey anti-mouse, and Alex Fluor 647 conjugated donkey anti-rabbit antibodies (Invitrogen, Grand Island, N.Y.).

For western blot analysis, the following antibodies were used: mouse anti-PARP (Trevigen, Gaithersburg, Md.), rabbit anti-NBS1 (Cell Signaling, Danvers, Mass.), rabbit anti-Rad50 (Cell Signaling, Danvers, Mass.), rabbit anti-mre11 (Gentex, San Antonio, Tex.), rabbit anti-rad51 (H-92, Santa Cruz Biotechnology, Santa Cruz, Calif.), mouse anti-p53 (DO-1, Santa Cruz Biotechnology, Santa Cruz, Calif.), and rabbit anti-BRCA2 (Calbiochem, Billerica, Mass.). Primary antibodies were detected using HRP-linked goat anti-rabbit or goat anti-mouse antibodies (Jackson Immunoresearch, West Grove, Pa.). GFP-supplemented Renilla luciferase containing plasmid pRL-TK (Promega, Madison, Wis.) was introduced in Hep3B and HepG2 cells using lentiviral infection. For generating an Mre11-knockdown cell line, GPIZ lentiviral shRNA (Open Biosystems, Lafayette, Colo.) was used according to the supplied protocol.

Media i.e. RPMI-1640 (for SNU449) or MEM (for HepG2 or Hep3B) was supplemented with 10% (v/v) fetal bovine serum. Additional supplementation for Hep3B culture media was performed with sodium pyruvate and non-essential amino acids. Cells were cultured in T-75 or T-150 tissue culture flasks (Corning Inc., Corning, N.Y.). For each cell line short tandem repeat fingerprint was confirmed by the Cell Line Characterization Core Service (M. D. Anderson Cancer Center, Houston, Tx.) within one year of all experiments. All media and supplements were purchased from Gibco (Life technologies, Grand Island, N.Y.). The cells were passaged approximately every three to five days before reaching confluency. Media was replaced every three days.

Before each experiment cells were counted. For clonogenic viability assays cells were counted using a hemocytometer and trypan blue staining as described in detail later. For all other assays, counting was performed using a cellometer (Nexcelom Bioscience, Lawrence, Mass.). First, cells were trypsinized, washed with and re-suspended in PBS. Approximately 20 μl of cell suspension was diluted 1:1 with trypan blue solution. Of the 40 μl, 20 μl was loaded on a disposable counting chamber (Nexcelom Bioscience, Lawrence, Mass.). The chamber slide was placed in the cellometer and viable cell counts were noted. For all experiments viability was recorded to be greater than 90%.

All reagents were purchased from Sigma Aldrich (Sigma-Aldrich Corp, St. Louis, Mo.) unless otherwise stated. Phosphate buffered saline was acquired from the surgical oncology core media facility (M.D. Anderson Cancer Center, Houston, Tex.). Bromodeoxyuridine (BrdU) was purchased from BD (BD Pharmingen, San Diego, Calif.). All experimental setups required 6-well or 12-well culture plates purchased from Corning Inc. (Corning, N.Y.).

For fluorescence microscopy, the following primary antibodies were used; rabbit anti-PAR (1:500, Trevigen, Gaithersburg, Md.), rat anti-RPA32 (1:200, 4E4, Cell Signaling, Danvers, Mass.), rabbit anti-Mre11 (1:500, GenTex, San Antonio, Tex.), rabbit anti-rad51 (1:100, H-92, Santa Cruz biotechnology, Santa Cruz, Calif.), mouse anti-γH2AX (1:1000, Upstate-Millipore, Billerica, Mass.), rat anti-BrdU (1:250, BU^{1/75}[ICR1], Abcam, Cambridge, Mass.). Primary antibodies were detected using the following secondary antibodies; Alex Fluor 488 conjugated donkey anti-rat, Alex Fluor 546 conjugated donkey anti-mouse and Alex Fluor 647 conjugated donkey anti-rabbit antibodies (both 1:500, Invitrogen, Grand Island, N.Y.).

Two transfections were performed. First, Hep3B and HepG2 cells were transfected to express GFP and luciferase to facilitate in vivo detection of xenografts. Renilla luciferase containing plasmid pRL-TK (Promega, Madison, Wis.) was modified by adding a GFP sequence. This recombinant plasmid was transfected into NIH 293T cells to generate a lentivirus vector containing the plasmid. This lentivirus was then used to infect Hep3B and HepG2 cell lines. GFP/Luc-transduced stable cells lines were obtained by sorting GFP-positive cells using FACScan (BD biosciences, Boston, Mass.)

For generating an Mre11-knockdown cell line, GPIZ lentiviral shRNA (Open biosystems, Lafayette, Colo.) was used according to the supplied protocol. In order to generate lentivirus, Hep3B cells were transfected in separate experiments with three random clones from a,shRNA library against Mre11 or a control shRNA together with a packaging plasmid (Trans-lentiviral packaging system, Open biosystems, Lafayette, Colo.) using lipofectamine 2000 (Invitrogen, Life technologies, Grand Island, N.Y.). Approximately 72 hours later, cells were observed under a microscope to express GFP, which is a marker of expression of shRNA against Mre11. Relative mre11 knockdown was confirmed by western blot analysis.

Immunocytochemistry

Approximately 50,000 cells were seeded in each well of a 12-well plate and grown on circular #1.5 cover slips (Electron Microscopy Sciences, Hatfield, Pa.). After 24 hours, cells were exposed to various treatment conditions as described herein. At the end of treatment, cells were fixed, permeabilized, blocked, labeled with primary antibody, and then with secondary antibody in consecutive steps. For confocal imaging, Fluoview—FV1000 Olympus Confocal Microscope (Center Valley, Pa.) was used.

For immunocytochemistry assay, an indirect immunofluorescence approach was used. Circular #1.5 cover slips (Electron microscopy sciences, Hatfield, Pa.) were placed in 12-well plates and sterilized using a 20-minute UV exposure. Cells from an exponentially growing culture were counted and approximately 50,000 cells were seeded in each well of a 12-well plate. Adherent sub-confluent monolayers were observed growing on the cover slip 24 hours later. At this point, cells were exposed to various treatment conditions as described herein. At the end of treatment, immunolabeling of proteins being studied was performed. Cells were fixed, permeablized, blocked, labeled with primary antibody and then with secondary antibody in consecutive steps. Between each step, cells were washed with PBS three times for 5 minutes each time on a leveled shaker at 50 RPM. Cells were fixed with 1% paraformaldehyde in PBS (w/v) for 30 minutes. Permeablization was performed using 0.3% (v/v) Triton-100 and 0.125% (w/v) CHAPS (3-[(3-Cholamidopropyl) dimethylammonio]-1-propanesulfonate) dissolved in PBS for 15 minutes. Cells were blocked for 1-hour in a 3% (w/v) bovine serum albumin and 1% (v/v) normal goat serum. Primary and secondary antibodies were diluted in the blocking buffer. Incubation with primary antibody was performed over night at 4° C. Secondary antibody incubations were performed for 2 hours at room temperature. At the end of immunolabeling, 4′,6-diamidino-2-phenylindole, DAPI (Molecular probes, Eugene, Oreg.) was used to counter stain DNA at 1:5000 dilution for 15 minutes. Cover slips were washed one more time with PBS and were mounted on frosted glass slides (Fischer Scientific, Waltham, Mass.) using Dako mounting media (Dako, Carpinteria, Calif.).

The slides were sealed with a conventional nail polish hardener and stored at 4° C. until imaging. For confocal imaging Fluoview—FV1000 Olympus Confocal Microscope (Center Valley, Pa.) was used. Images were acquired using a 60× (NA1.6, oil) or 100× (NA1.3, oil) objective at an X-Y resolution of 100 nm and a Z-stack resolution of ˜800 nm. Samples were excited using an incident LD laser at 405 nm (50 mW, 5% power) for DAPI, 559 nm (15 mW, 20%) for Alex Fluor 546, and 635 nm (20 mW, 20% power) for Alex Fluor 647. Capture resolution was set at twice the optical resolution and Kallman averaging was set at 2 for enhanced signal to noise ratio. Images were acquired sequentially to minimize cross contamination from multiple emission spectra. Exposure settings were set to maximize dynamic range initially and then kept constant across multiple samples to allow quantitative comparisons.

Acquired images were processed in Slidebook (version 5.0, Intelligent Imaging Innovations, Inc., Denver, Colo.). Nuclei were identified using the DAPI channel and the areas were converted to regions of interest (ROI). Signals in these ROIs from other channels were used for colocalization analysis. Colocalization thresholds were defined using control images from secondary antibody alone slides such that less than 5% of the pixels exceeded this threshold. Pearson's correlation was used as an index of colocalization of pixels with intensity above the background threshold.

Immunohistochemistry

Tumor tissues harvested at the end of the experiment were fixed in 10% buffered formalin (pH 7.0) for 24 hours and subsequently stored in 70% ethanol (v/v) before embedding them in paraffin. For immunohistochemistry, 5-micron sections were placed on a glass slide and tissue sections were de-paraffinized and rehydrated. Antigen retrieval was performed in citrate buffer at pH 6.0. The slides were placed in Tris-buffer (pH 8.0) before further processing.

For fluorescence immunohistochemistry, a protocol similar to that used for immunocytochemistry (described above) was used with some modifications described here. Tris-buffer was removed and tumor section was encircled using a pap pen (Electron microscopy sciences, Hatfield, Pa.). For blocking, 5% (w/v) BSA and 3% (v/v) NGS was used instead of 3% BSA and 1% NGS, respectively. Imaging was performed as for immunocytochemistry.

For chromogen-based immunohistochemistry, cleaved caspase-3 (CC-3) and LC3B protein were detected using a rabbit monoclonal antibody (CC-3, 1:1000; LC3B 1:400, Cell Signaling, Danvers, Mass.), and Ki67 was detected using a mouse monoclonal antibody (1:150, Dako, Carpinteria, Calif.). Mouse antibody was detected using mouse-on-mouse HRP-Polymer Kit (BioCare Medical, Concord, Calif.). Rabbit antibody was detected using EnVision+/HRP, rabbit kit (Dako, Carpinteria, Calif.). The staining was performed on a Dako automated stainer (Dako, Carpinteria, Calif.). Primary antibody incubation was 30 minutes. The slides were counterstained with hematoxylin and a cover slip was sealed in place. Images were acquired using a multispectral scope (Olympus IX51 featuring a CRi Nuance camera, Hopkinton, Mass.). Staining was quantified using inForm™ (CRI, Capillary Life Sciences, Hopkinton, Mass.), pattern recognition software that subjects multispectral data to machine-learning algorithms for accurate quantification of staining.

Clonogenic Assays

Clonogenic assay estimates single-cell reproductive viability by measuring the ability of a single cell to form a colony of 50 cells or more. Clonogenic assay in this study was performed as described previously (Franken et al., 2006). Briefly, cancer cells from an exponentially growing, sub-confluent culture were trypsinized and harvested. Approximately 200 cells were counted and plated in each well of a 6-well plate. Approximately 12 hours later, the cells were adherent and treatment conditions were introduced as described herein. After varying treatments, media was replaced and cells were allowed 14 days to form colonies. Subsequently, the media was aspirated and cells were washed with PBS once. Colonies were then fixed with glutaraldehyde (6.0% v/v) and stained with crystal violet (0.5% w/v) for 30 minutes on a leveled shaker at 50 RPM. The fixation and staining solution was then aspirated and the 6-well plates were air-dried after gentle rinsing with tap water. The number of colonies in each well was counted using a colony counting grid. Early passage HepG2 cells failed to form colonies using standard conditions or by using pre-conditioned media from the exponentially growing HepG2 culture. Therefore, a clonogenic assay on HepG2 cells could not be performed.

Cell Cycle Analysis

For cell cycle analysis a BrdU-labeling protocol from BrdU Flow Kit (BD Pharmingen, San Diego, Calif.) was used according to the supplied instructions. Data was analyzed using FlowJo 7.63 (Tree Star, Inc., Ashland, Oreg.).

For cell cycle analysis, a BrdU-labeling protocol was used to identify cells in S-phase of the cell cycle accurately. Cells were harvested from an exponentially growing culture as described for other experiments and seeded in 6-well plates, approximately 150,000 cells per well. After 24-hours adherent sub-confluent monolayers were noted and cells were exposed to various experimental conditions. At the end of the treatments, cells were washed and sampled at various time points to study the progression of the cell cycle in time. One hour before each time point, cells were pulsed with BrdU (10 μM) for 1 hour. BrdU gets incorporated in the DNA of cells in S-phase along with other nucleotides and can be detected using a mouse FITC conjugated anti-BrdU antibody. A BrdU Flow Kit (BD Pharmingen, San Diego, Calif.) was used according to the supplied instructions without modifications. For counterstaining of DNA content, cells were incubated with 7-amino-actinomycin D (7-AAD) supplied with the kit, on ice for 20 minutes before analysis on a BD LSR II flowcytometer (BD biosciences San Jose, Calif.).

Single cell populations were identified using forward and side-scatter profiles. A total of 10,000 events were recorded from a gated single cell population. For 7-AAD fluorescence measurements, a SORP YG laser (561 nm) was used to excite the cells and emission was recorded through a 630LP filter followed by a 660/20 filter. For FITC fluorescence measurements, a SORP blue laser (488 nm) was used to excite the cells and emission was recorded through 505LP filter followed by a 525/50 filter. There was no spectral over-lap between the two emission spectra and compensation was not required. Data was analyzed using FlowJo 7.63 (Tree Star, Inc., Ashland, Oreg.).

Mouse Model of Hepatocellular Carcinoma

For in vivo studies, an implanted mouse model of human HCC was generated in CB17SCID mice (Taconic, Hudson, N.Y.). Female mice between 4 and 5 weeks in age were purchased and acclimatized in M.D. Anderson Animal facilities for up to one week. All animals were handled, housed and studied in accordance with the Institutional Animal Care and Use Committee. Tumors were generated after orthotopic implantation of approximately 1.6 million cells into the liver of each mouse (10 mice per group). Bioluminescence imaging indicated tumor development in the majority of mice three weeks after implantation. Euthanasia was performed by carbon dioxide inhalation followed by cervical spine dislocation.

Cultured luciferase and GFP-expression human HCC cell lines (Hep3B and HepG2) were harvested from an exponentially growing culture and washed with PBS. The cells were re-suspended in 3 ml PBS and final centrifugation was performed at 1000 RPM for 5 minutes. Supernatant was discarded and the cell pellet was used for implantation. The concentration of cells achieved using this protocol was 160,000 cells per micro liter. The cells were kept on ice and animals were injected within 3-4 hours after final centrifugation.

Before surgery, hair was removed from the ventral surface of the abdomen using clippers. Mice were anesthetized using 2.5% isoflurane. The surgical field was sterilized with 70% (v/v) ethanol in water. Mice were placed supine on a heating pad and the surgical site was sterilized using povidone iodine swab sticks. After confirming induction of anesthesia, an approximately 1-cm transverse incision was made in the skin of the upper abdomen slightly left of the midline. Deeper layers of muscle and peritoneum were incised and hemostasis was achieved using silver nitrate chemical cautery sticks. By applying gentle pressure on the lower abdomen and lower chest, the left lobe of the liver was eviscerated. A 10 μl volume of cells was aspirated (˜1.6 million cells) using a Hamilton syringe with an angled 30-guage-needle tip (point style 4, 30°, Hamilton Company, Reno, Nev.). The needle was advanced ˜5 mm in to the liver parenchyma of the left lobe and the cells were gently deposited ˜2 mm underneath the liver capsule such that a bleb of fluid was observed. The needle was carefully withdrawn and the needle track was immediately compressed with a sterilized cotton Q-tip for ˜60 seconds. A dab of silver nitrate cautery or super glue was used to ensure hemostasis at the needle site. The liver was returned to the peritoneal cavity. Peritoneum, abdomen and skin were closed in a single layer using stainless steel surgical clips (Harvard Apparatus, Holliston, Mass.). After surgery the mice were allowed to regain consciousness under a thermal lamp and observed for 30-60 minutes before returning them to the housing. Staples were removed 10-14 days after surgery.

Three weeks after implantation of tumor cells in the liver, bioluminescence measurements were performed. D-Luciferin from firefly (Caliper Life Sciences, Hopkinton, Mass.) was administered at a dose of 150 mg/kg in 100 μl intra-peritoneally (i.p.). Animals were anesthetized using 2.5% isoflurane and imaged using Xenogen IVIS-200 (Caliper Life Sciences, Hopkinton, Mass.) 5 minutes after the injection. The imaging was performed over 2 minutes with a 1×1 binning. Mice that had any bioluminescence activity above background (suggesting the development of tumors) were included in the subsequent study. Based on bioluminescence ˜95-99% of mice develop tumors 3 weeks after implantation of cells for both tumor models. While bioluminescence imaging was useful in determining the presence of tumors before the start of the study, the levels of bioluminescence did not correlate with the size of the tumors and hence could not be used to track the growth of tumors during the study.

Mice with confirmed tumors on bioluminescence were then randomized in groups of 10. Randomization was blinded as the tumors were not visible to naked eye and the investigator performing the randomization was separate from the investigator administering treatments. Sample size was calculated using Stata SE 12.1 for Mac (College Station, Tex.). Following parameters were used: Effect size: 15% mass reduction any two groups, Power: 0.90, Standard deviation: 10%, Alpha: 0.05

Radiofrequency Generator Setup

A non-invasive external RF generator (ThermMed, LLC, Erie, Pa.) was used for animal hyperthermia exposures. The use of this generator has been described previously (Glazer et al., 2010). The generator operates at an adjustable output power (0-2 kW) at a fixed frequency, 13.56 MHz. The generator is connected to a high Q coupling system with a TX head (focused end-fired antenna circuit) and reciprocal RX head (as a return for the generator) mounted on a swivel bracket allowing the RF field to be oriented in either a horizontal or vertical direction. The two heads were set at a distance of 3.5 inches apart. The TX head is covered with a Teflon plate whereas the RX head has a conducting copper surface to allow grounding of the animals as described later. The coaxial end-fire circuit in the TX head produces a uniform RF electric field up to 15 cm in diameter. The field generated is predominantly electric with minimal magnetic component. Attempts to measure this electric field previously using a Hewlett Packard Spectrum Analyzer (model 8566B, Agilent, Santa Clara, Calif.), an isotropic field monitor and a probe (models FM2004 and FP2000, Amplifier Research Inc., Souderton, Pa.) were not successful. However, at high power output (>100 W) accurate measurements cannot be performed because of heating of the measurement probe itself

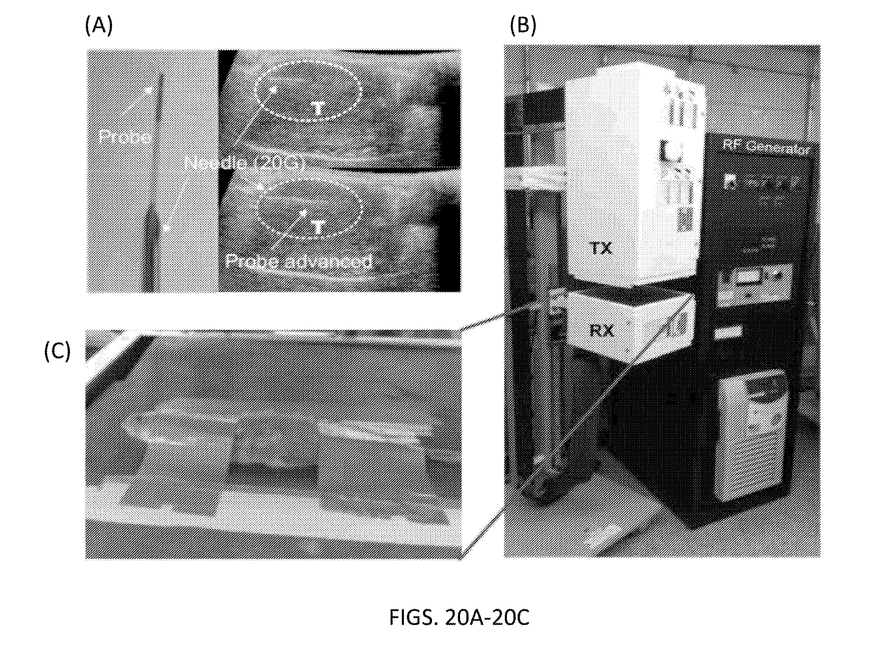

For animal RF field exposures, mice were anesthetized with a cocktail of ketamine (100 mg/kg i.p.) and xyalzine (10 mg/kg i.p.). Hairs were removed from the anterior abdominal wall using clippers. Before administering RF exposures, certain steps were taken to ensure prevention of electrothermal injury. For instance, mice that urinate in the RF field suffer severe burns in the groin region. This is because urine with high ionic content heats significantly faster than the rest of the mouse in an RF-field. By gently pressing on the lower abdomen of the mouse, urine was removed from the bladder before RF exposures. Parts of a mouse's body with pointed geometry (paws, limbs, ears, whiskers and tail) accumulate a very high charge at the tips because of an impedance mismatch. As a result, mice can suffer intense electrothermal burns at these sites. The experiments were, therefore, performed after grounding all parts of the mouse's body using a copper tape. A window was created within the copper tape grounding-shield to allow RF exposure to the abdomen (FIG. 20). All experiments were performed for a 10-minute duration at 600 W power output to be consistent with prior reports in the literature (Glazer et al., 2010).

Statistical Analyses

The data were plotted and analyzed in GraphPad Prism (version 5, La Jolla, Calif.). For data with Gaussian distribution and when comparing two groups, the Student unpaired two-sided t-test was used. Multiple group data were analyzed using one-way analysis of variance (ANOVA). Where necessary, ad-hoc post-tests were performed, and the type of test used is reported with the results. For all inferential statistics a P value<0.05 was considered significant. All statistical tests were unpaired two sided unless noted otherwise.

Protein Electrophoresis and Immunoblotting

Approximately 200,000 cells were plated in 60 mm cell culture plates. Cells formed sub-confluent monolayers. The cells were exposed to moderate hyperthermia in an incubator at 42.5° C. for 2 hours and cell lysates were prepared for western blotting before and at 0, 1, 4 and 24-hours after hyperthermia exposure to evaluate the relative levels of various proteins. For preparation of whole cell lysates, media was removed and cells were washed with PBS. Cell lysis buffer (150 mM NaCl, 1% NP-40, 0.5% sodium deoxycholate, 0.1% SDS, 50 mM Tris,pH 8.0, one mini protease inhibitor/10 ml tablet Rosche #11 836 153001) was added on ice and lysed cells were collected after gentle scraping and mixed on ice for 30 min. The lysates were spun at 15000 RPM for 10 min at 4° C. The supernatant was collected and stored at −80° C. before electrophoresis.

Protein concentrations were measured using a Bradford assay (Fischer scientific, Pittsburgh, Pa.) with bovine serum albumin as the standard curve, according to the manufacturer's instructions. The samples were loaded at 20-40 μg/well in a gel using a denaturing sodium dodecyl sulphate-polyacrylamide gel electrophoresis (SDS-PAGE) system. Electrophoresed proteins were transferred onto a polyvinylidene fluoride (PVDF) membrane overnight on ice. The transferred proteins were probed using a specific antibody against each protein of interest. The primary antibody was detected using a horseradish peroxidase (HRP)-conjugated secondary antibody. HRP-conjugated secondary antibody was detected using an Amersham ECL detection system (GE Health Care Biosciences, Pittsburgh, Pa.).

For BRCA 2 detection, cell lysates were prepared as for other proteins. However, a NuPAGE large protein blotting kit was used that utilizes a 3-8% tris-acetate gradient gel allowing better resolution of larger proteins.

Thermal Imaging and Fiber Optic Thermography

During RF-field exposure, temperature from the abdominal surface of mice was recorded using an infrared thermal camera (FLIR SC 6000, FLIR Systems, Inc., Boston, Mass.). This non-invasive measurement was performed for all mice to ensure that the surface temperature did not exceed 43° C.

For liver and orthotopic xenograft measurements fiber optic thermography was employed. Fluotemp, a fiber-optic probe (PhotonControl, Burnaby, BC, Canada), 400 microns in diameter with a scientific accuracy of 0.1±° C. was advanced over a 20G, 1-inch needle. The needle was placed in the liver or liver tumor under ultrasound guidance and the probe was advanced into the target organ. Subsequently, the needle was retracted over the fiber-optic probe. This probe was pre-tested for lack of heating in the RF field.

Animal Model of Human HCC and RF-Hyperthermia

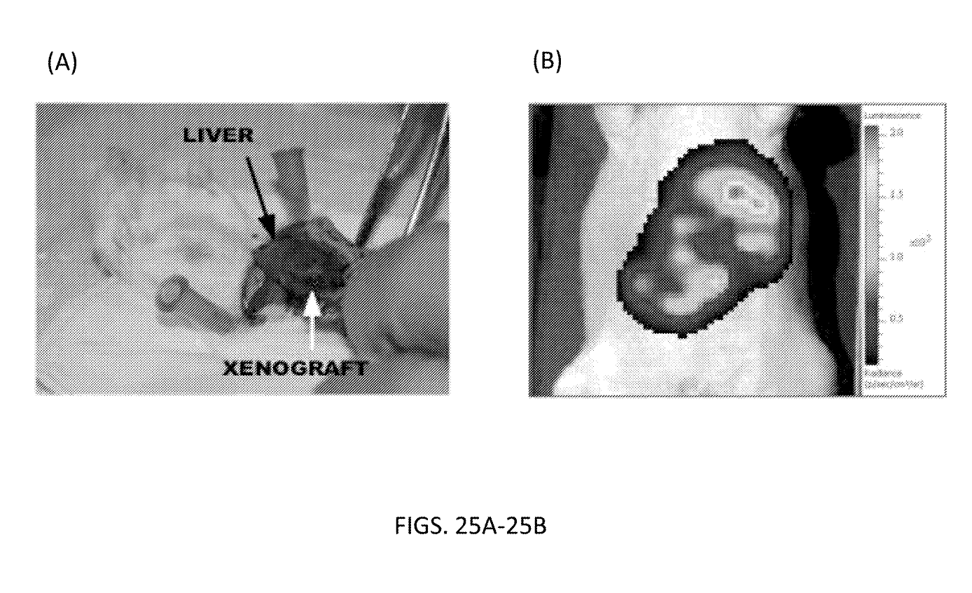

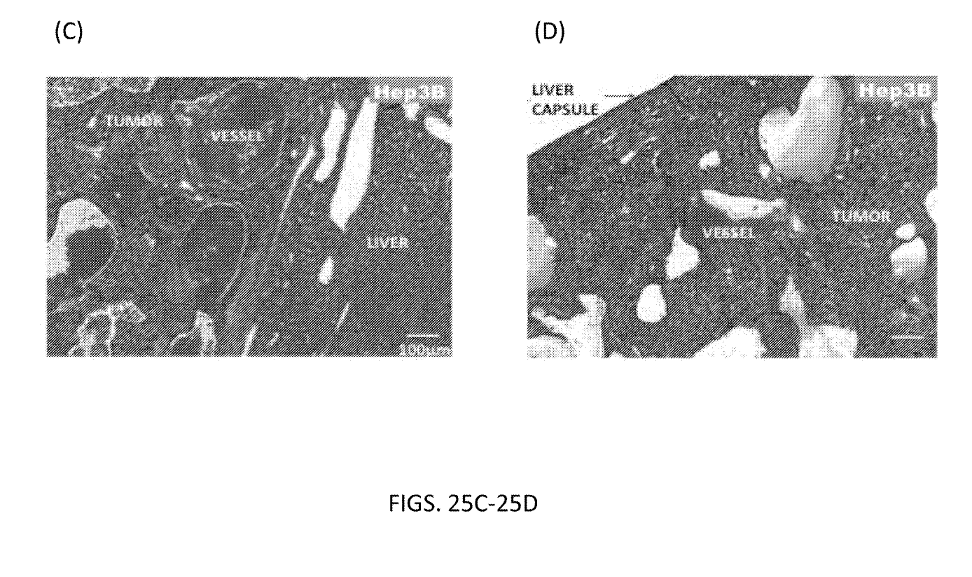

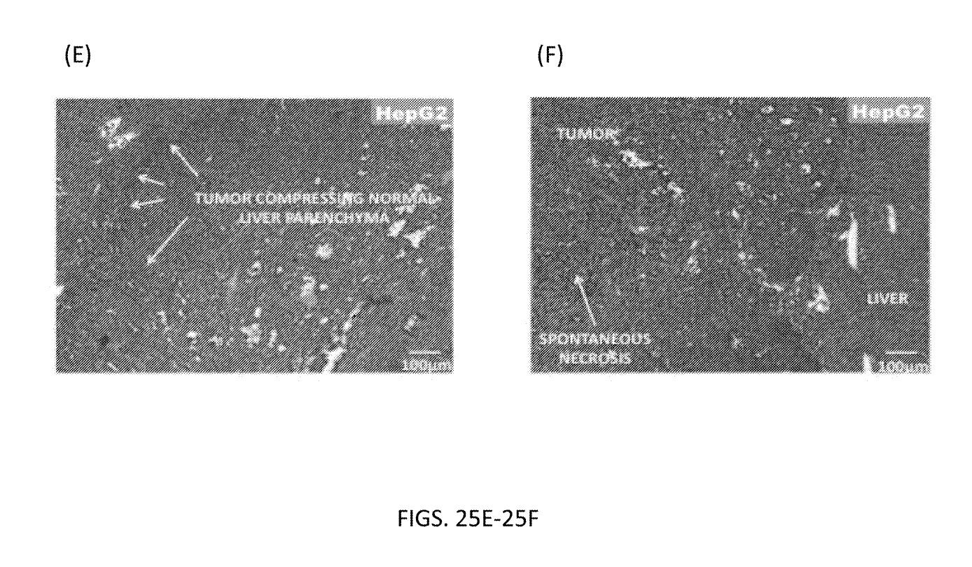

Two animal models of human primary hepatocellular carcinoma were developed using a wt-p53 HepG2 cell line or null-p53 Hep3B cell line in immune-deficient CB17 SCID mice. The cells were implanted in the liver to generate an orthotopic tumor model as detailed in the methods. Both xenografts were locally aggressive, eroded (as opposed to invaded) the normal adjacent mouse liver and had no distant or intra-hepatic metastasis or extension in non-liver viscera. However, Hep3B xenografts were fast growing unlike the HepG2 xenografts. On histological analysis, both tumors appeared hyper-vascular with areas of spontaneous necrosis (FIG. 25). There were larger aberrant vessels with Hep3B xenografts, which were not seen with HepG2 xenografts. The xenograft models closely mimicked non-metastatic human primary hepatocellular carcinoma based on growth pattern, macroscopic and microscopic appearance.

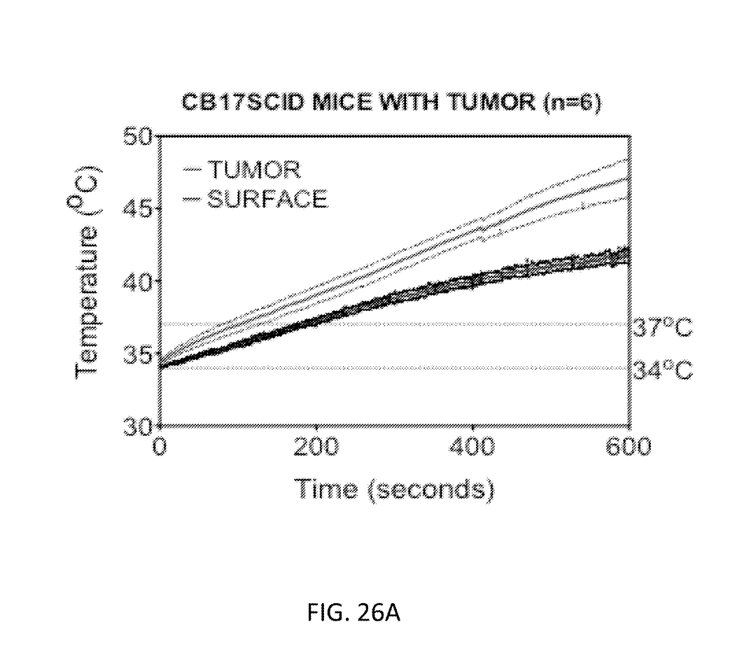

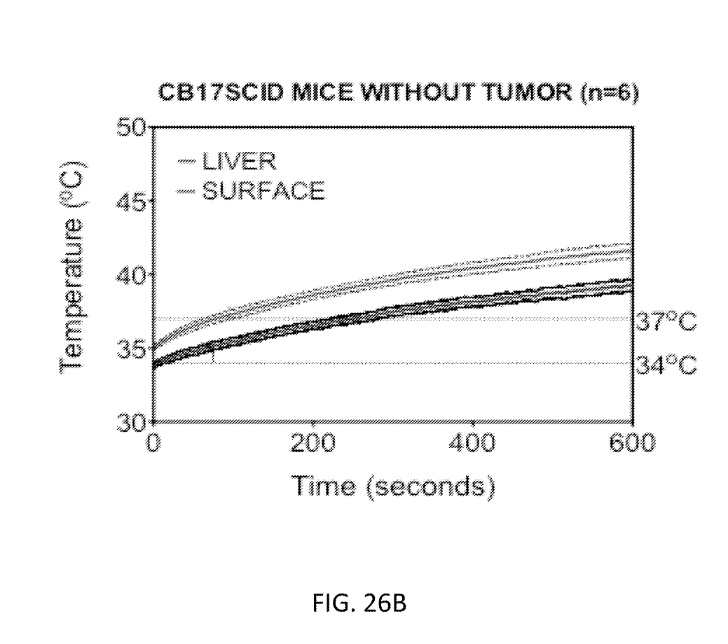

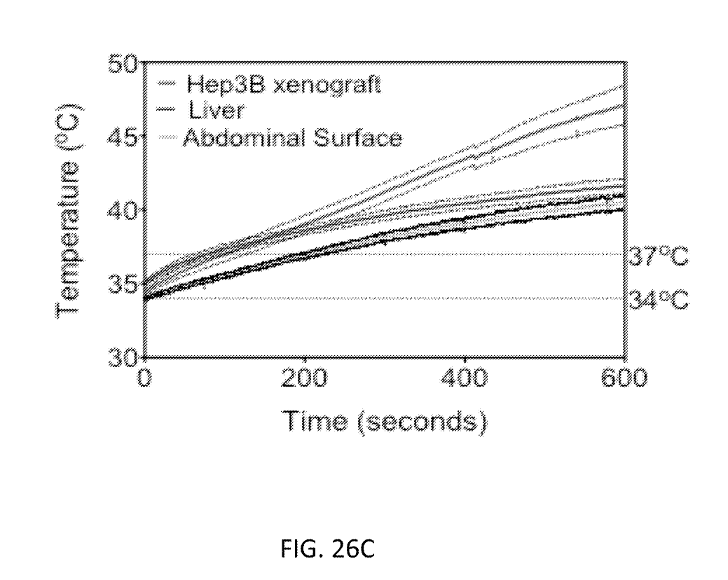

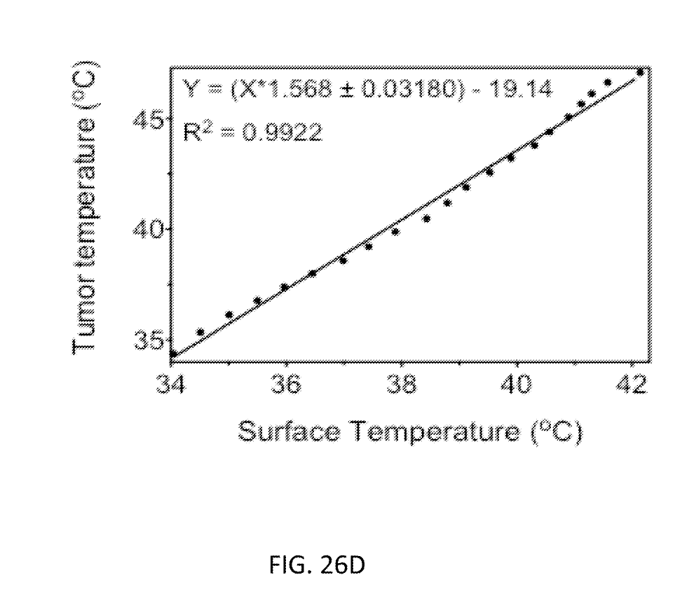

After establishing the tumor models, it was evaluated if hyperthermia could be successfully delivered to the tumor tissue using the 13.56 MHz non-invasive Kanzius RF generator. For that purpose, fiber-optic thermography was employed to measure the temperature of liver tumor and normal liver in anesthetized mice subjected to RF field exposure for 10 min at 600 W. Since fiberoptic thermography requires placement of a fiber-optic (400 micron diameter) probe under ultrasound guidance and is challenging for smaller tumors, we wanted to investigate if abdominal surface temperature as measured by infrared thermal imaging correlated with tumor temperature. This would allow one to monitor thermal dose in real-time non-invasively for future experiments. The data are shown in FIG. 26.

After anesthesia and during placement of mice on the RX head of the RF generator, a drop in core body and surface temperature was noted. For consistency, we allowed the abdominal surface temperature to drop to 34° C. before starting RF exposures. During a 10-minute RF exposure there was a duration-dependent near-linear rise in Hep3B xenograft and normal liver temperature. Interestingly, normal livers heated significantly less than Hep3B xenografts, suggesting, some tumor selective heating effect of RF-field exposure. Abdominal surface temperatures recorded during these experiments demonstrated a strong linear correlation with tumor temperature (R2=0.99). Prior reports have demonstrated that a 10-minute RF exposure using the same parameters is safe with no detectable harm to normal tissues (Glazer et al. 2010). Here it is shown that the same RF exposure can be used to deliver tumor selective hyperthermia to orthotopic liver tumors. However, the underlying reason for tumor selective hyperthermia is not evident from these experiments. Thermal dose calculations as defined by cumulative equivalent minutes at 43° C. (CEM43) were performed using the average time-temperature plots obtained from liver tumor and normal liver during a 10-minute RF exposure. CEM43 for liver tumor was ˜80 fold higher than that of normal liver (CEM43: 1401.6 vs. 17.5).

Effect of Hyperthermia on HRR-Pathway Proteins

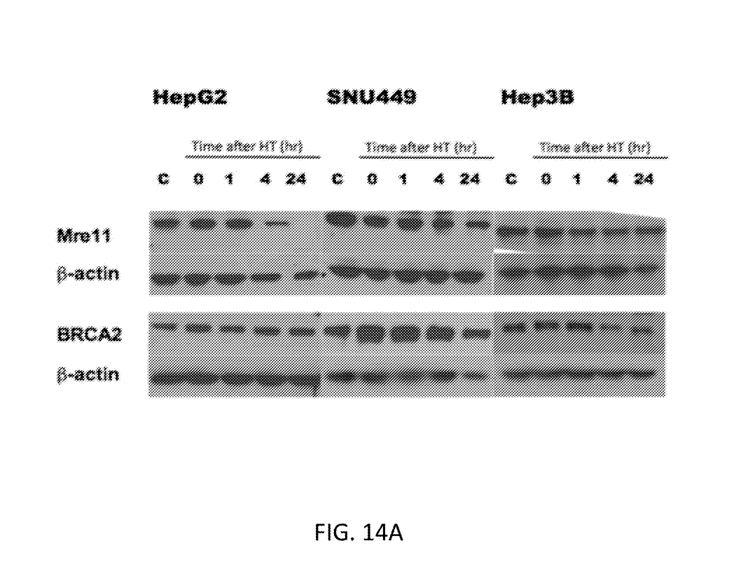

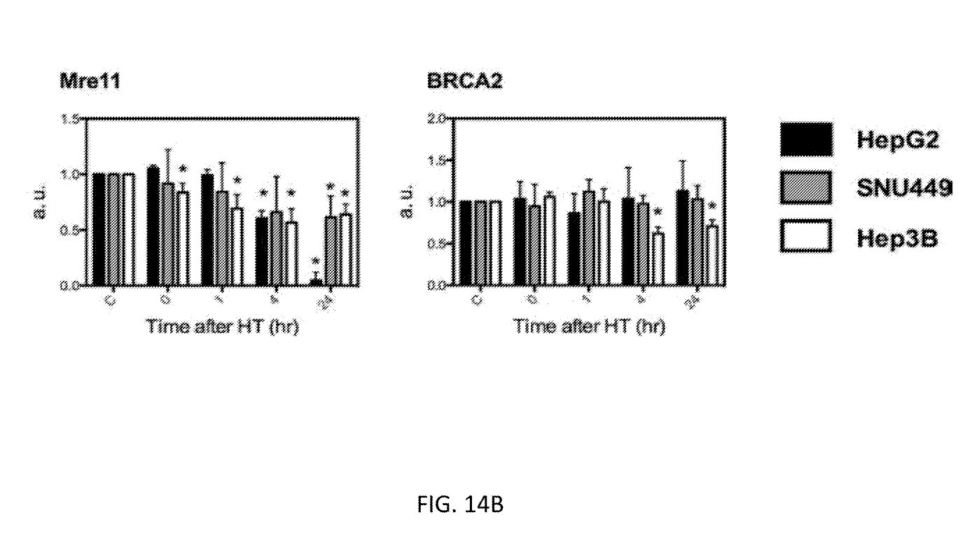

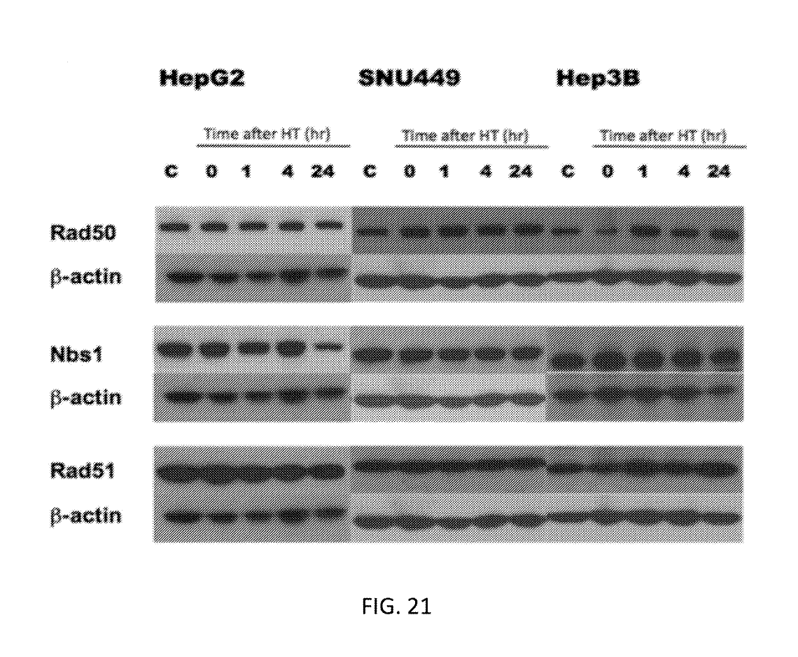

To investigate the effects of hyperthermia on HRR-pathway proteins, three different human HCC cells lines, i.e., Hep3B, HepG2, and SNU449, were evaluated. Cells were subjected to hyperthermia at 42.5° C. for two hours in an incubator. Protein expression levels were monitored at varying time points. The data are shown in FIGS. 14A and 14B, and FIG. 21.

With hyperthermia exposure, levels of Nbs1, Rad50 and Rad51 showed minimal change. The levels of Mre11 gradually declined after heat shock in all cell lines to less than half of the control levels at 24 hours, i.e., normalized mean absorbance units ±SD at 24 hours for Hep3B cell line were 0.64±0.09, P=0.0024; for HepG2 cell line was 0.45±0.07, P<0.0001; and for SNU449 cell line was 0.61±0.19, P=0.024, compared with respective control groups. It has been previously reported that BRCA2 is an important target of heat radiosensitization (Krawczyk, et al., 2011). The effect of hyperthermia on BRCA2 levels was cell-line-dependent. The HepG2 cells and SNU449 cells demonstrated negligible changes in BRCA2 levels after thermal exposure (statistically not significant); however, Hep3B cells demonstrated a slight decrease in BRCA2 levels, i.e., normalized mean absorbance units ±SD at 24 hours for Hep3B cell line were 0.70±0.13, P=0.02, compared with the control group.

Localization of HRR-Pathway Proteins to Gemcitabine-Stalled Replication Forks

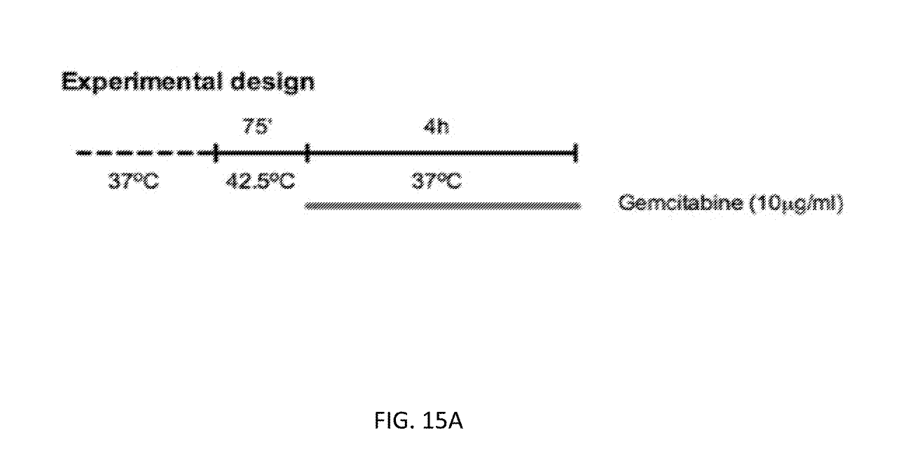

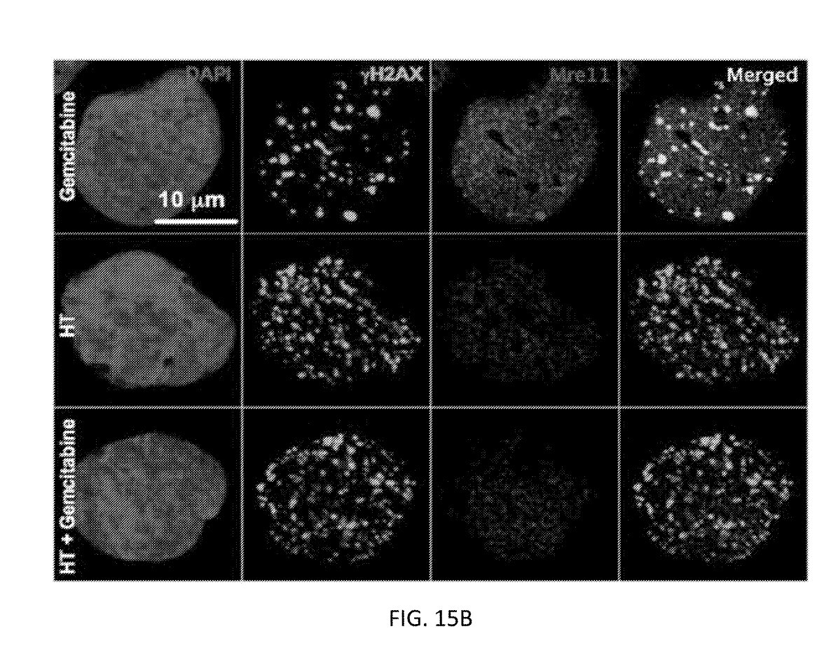

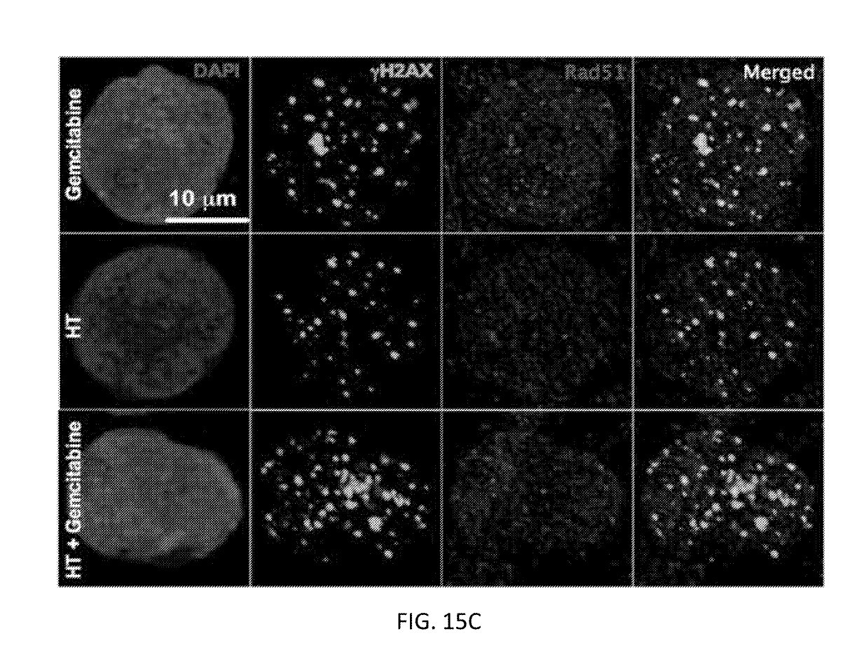

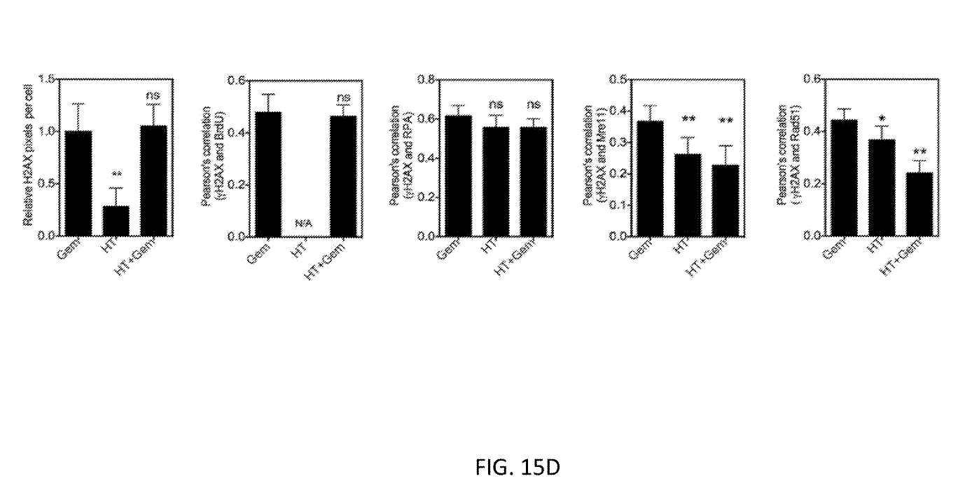

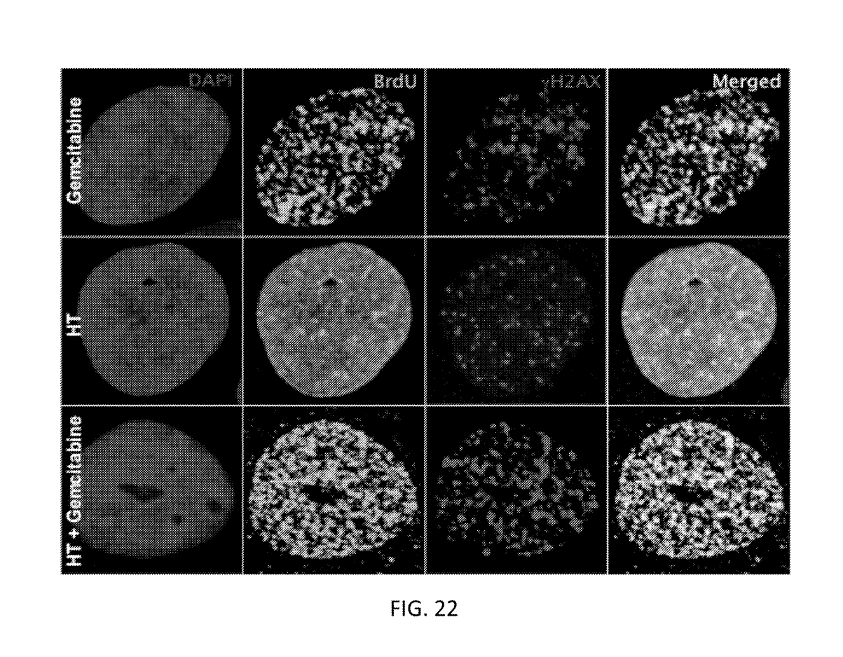

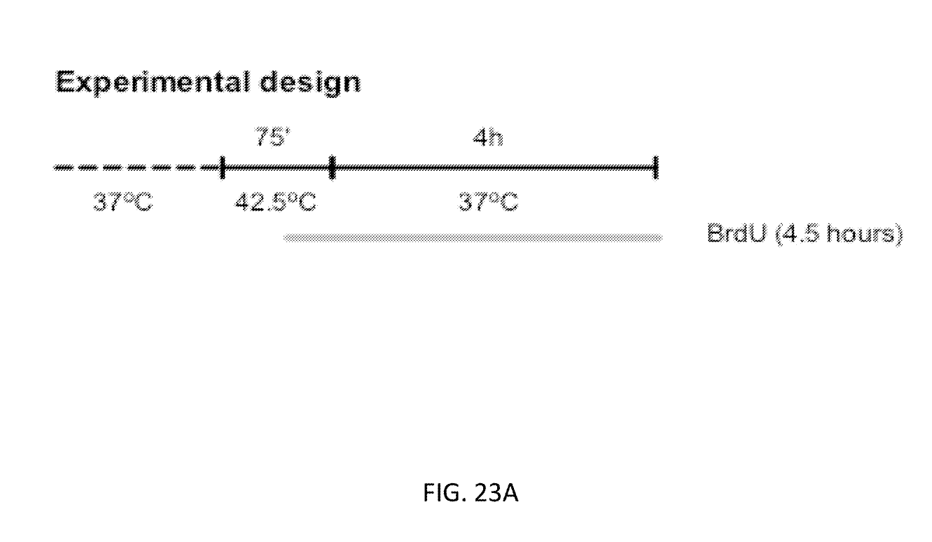

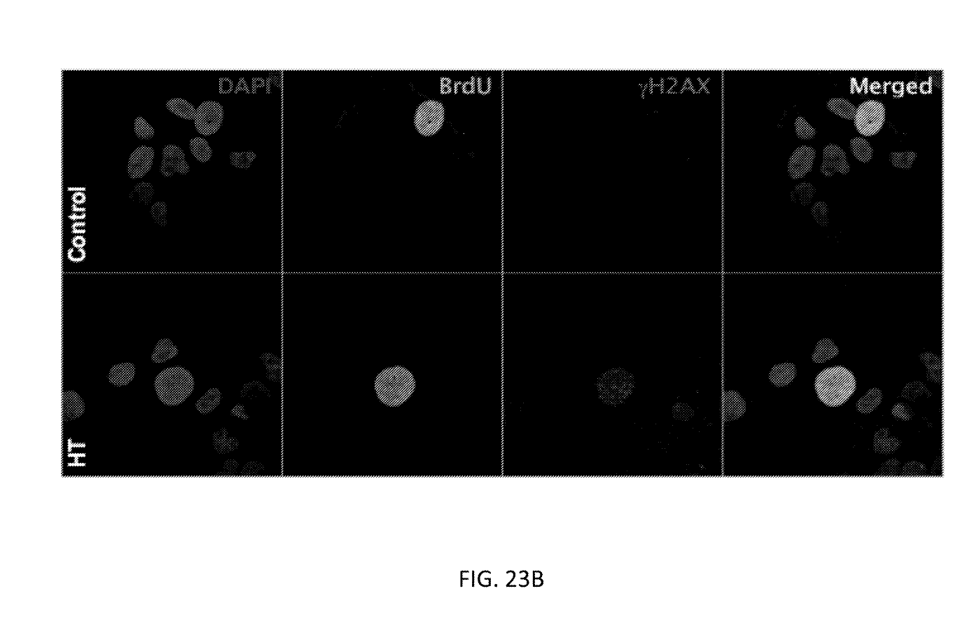

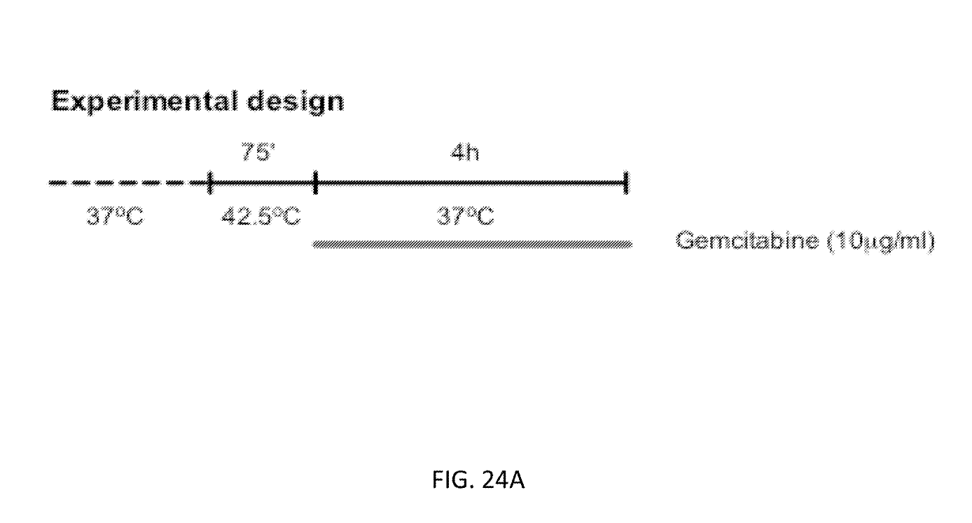

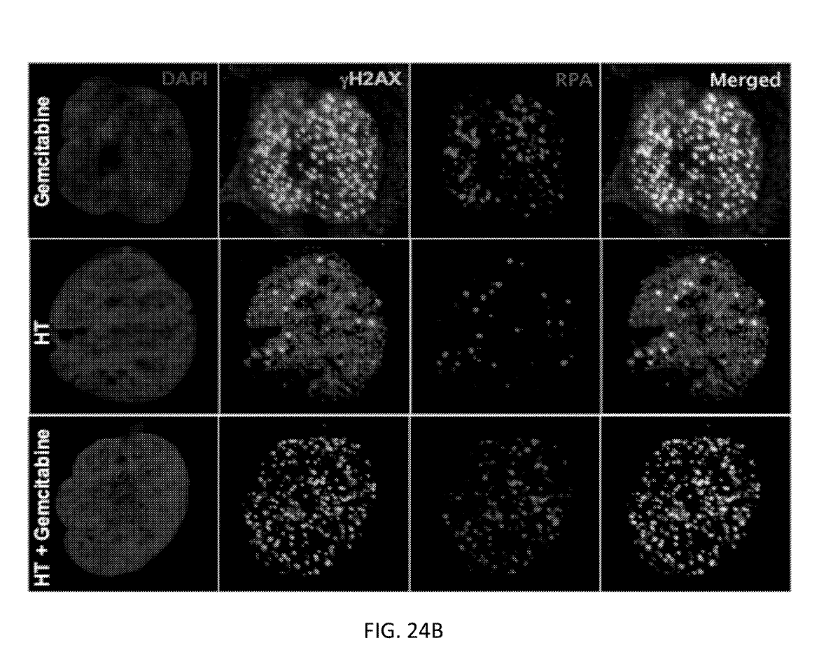

γH2AX-p as a marker of stalled replication forks was investigated. Hep3B cells were pulsed with BrdU for 30 minutes prior to addition of gemcitabine. This allowed one to label the DNA just downstream to the stalled replication fork site. When these cells were labeled with γ-H2AX antibody, almost all γ-H2AX foci localize with BrdU foci, confirming their presence at stalled replication forks (FIG. 22). Cells that were treated with hyperthermia alone also demonstrated γ-H2AX foci. However, the relative mean (±SD) numbers of γ-H2AX pixels per cell were statistically significantly fewer than those in cells treated with gemcitabine alone (0.28±0.17 vs 1.0±0.26, P=0.017, Student's t-test, FIG. 15D). The γ-H2AX foci were only found in cells positive for BrdU, suggesting specificity for S-phase (FIG. 23). Of note, in control experiments (i.e., cells where BrdU label was added but no hyperthermia or gemcitabine treatment was given), γ-H2AX foci could not be found for further quantification (FIG. 23). Hyperthermia immediately followed by gemcitabine treatment did not increase mean (±SD) γ-H2AX pixels per cell compared with those in cells treated with gemcitabine alone (1.0±0.20 vs 1.0±0.26, P=0.8, Student's t-test, FIG. 15D). This suggests a common etiology to the origin of γ-H2AX foci with hyperthermia, i.e., stalled replication forks. Transient stalling of replication forks is known to occur with hyperthermia, based on prior reports (Warters, et al., 1983). If this is correct, hyperthermia induced-γ-H2AX foci should colocalize with single-stranded DNA resulting from stalled replication forks. Using replication protein A (RPA) as a marker of single-stranded DNA, γ-H2AX foci after hyperthermia exclusively colocalize with RPA foci. This confirms that γ-H2AX phosphorylation after hyperthermia corresponds to sites of single-stranded DNA (FIG. 24).

After establishing γ-H2AX foci as a marker of stalled replication forks, recruitment of downstream pathway proteins to these sites was evaluated. Cells were exposed to mild hyperthermia at 42.5° C. for 75 minutes followed immediately by a high concentration of gemcitabine (10 μg/ml). This concentration was chosen to induce maximal stalling of replication forks and hence to produce the maximum number of γ-H2AX foci in each cell. Stalling leads to stretches of single-stranded DNA. Replication protein A (RPA) has a high affinity for single stranded DNA and is important for recruitment of downstream HRR-pathway proteins to stalled replication forks. Pretreatment with hyperthermia does not inhibit localization of RPA to stalled replication forks (FIG. 24). Downstream, RPA recruits proteins of the Mrell-Rad50-Nbs1 (MRN) complex. Within this complex, Mre11 is the key effector with known 3′ to 5′ exonuclease as well as 5′ to 3′ endonuclease activity. In particular, Mre11 nucleolytic activity allows loading of Rad51 recombinase by processing DNA ends at stalled replication forks. Pretreatment with mild hyperthermia decreases overall levels of Mre11 as well as impairs localization of Mre11 to sites with γ-H2AX foci; mean Pearson correlation for the gemcitabine and hyperthermia group (0.23, 95% CI=0.16 to 0.29) was statistically significantly lower than that of the gemcitabine alone group (0.37, 95% CI=0.32 to 0.42, P=0.0008, FIGS. 15B and 15D). This pretreatment further leads to inhibition of Rad51 loading at stalled replication sites; mean Pearson correlation for the gemcitabine and hyperthermia group, (0.44, 95% CI=0.40 to 0.48), was statistically significantly lower than that of the gemcitabine alone group (0.24, 95% CI=0.19 to 0.29, P<0.0001, FIGS. 15C and 15D).

These data demonstrate that hyperthermia alters localization of the HRR-pathway proteins at sites of stalled replication forks. Mrell is a key thermolabile target of hyperthermia.

Cell Cycle Alterations

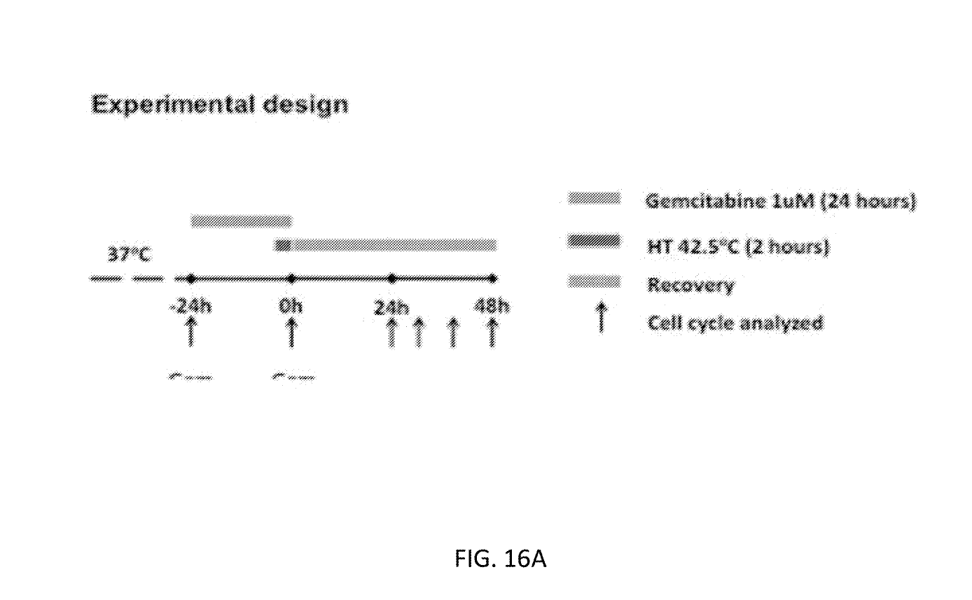

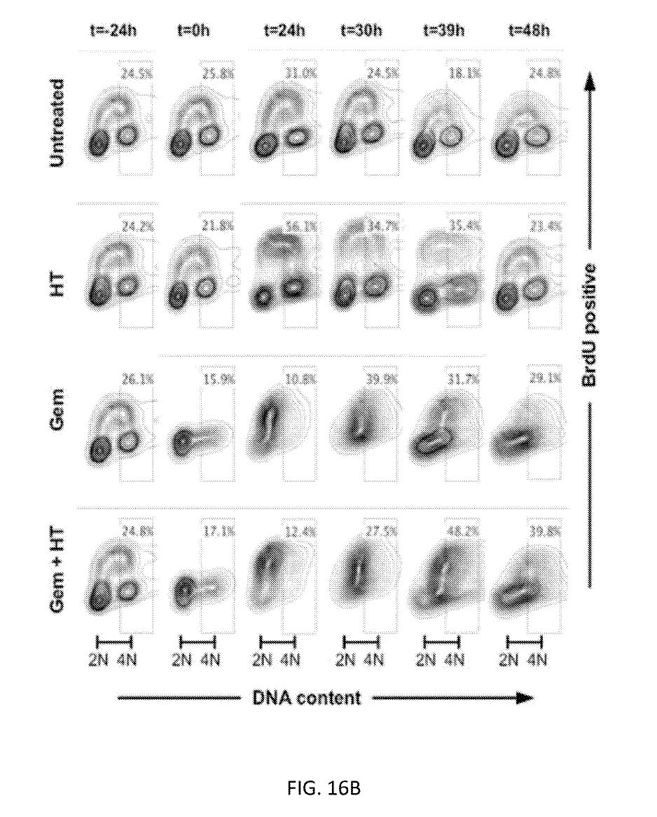

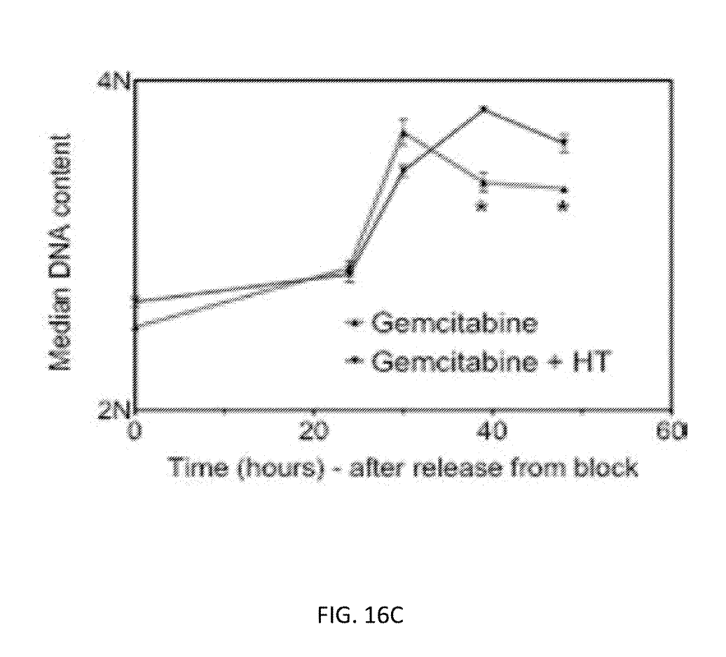

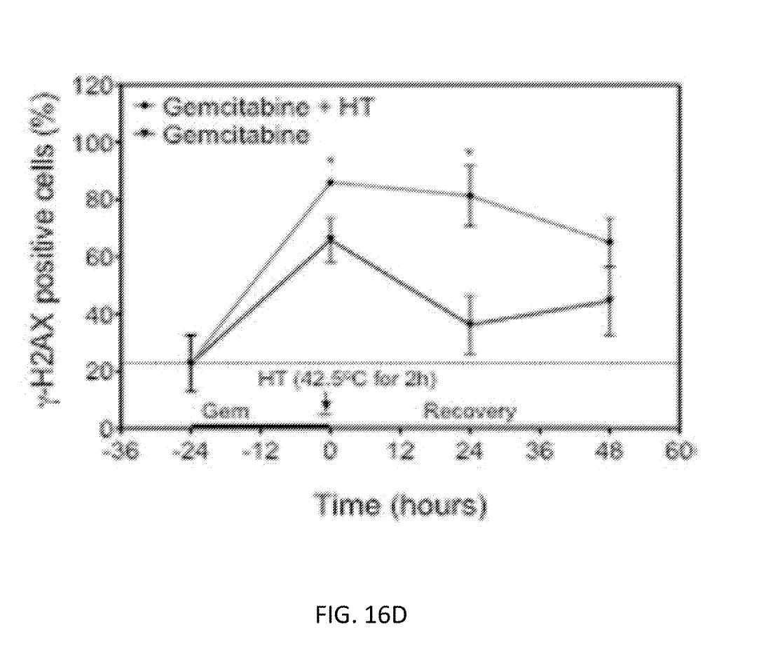

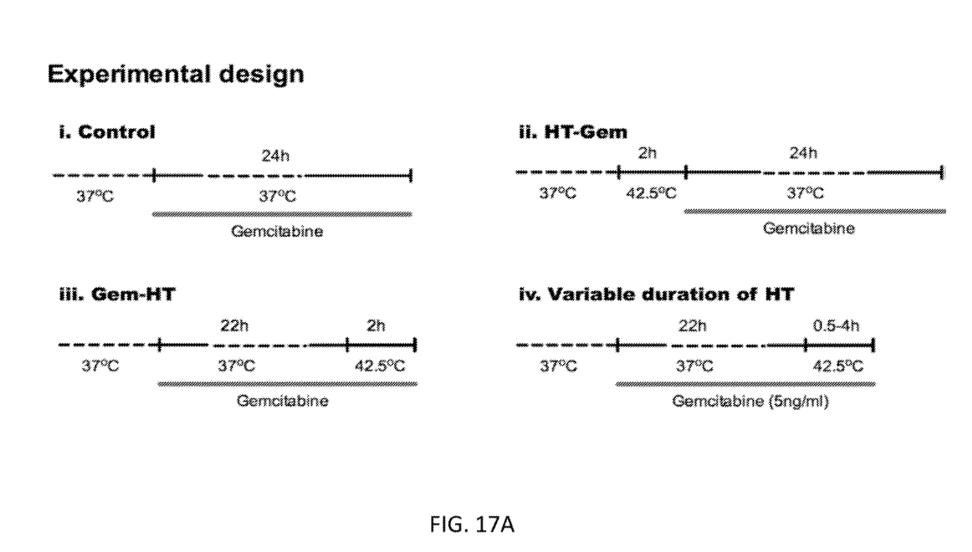

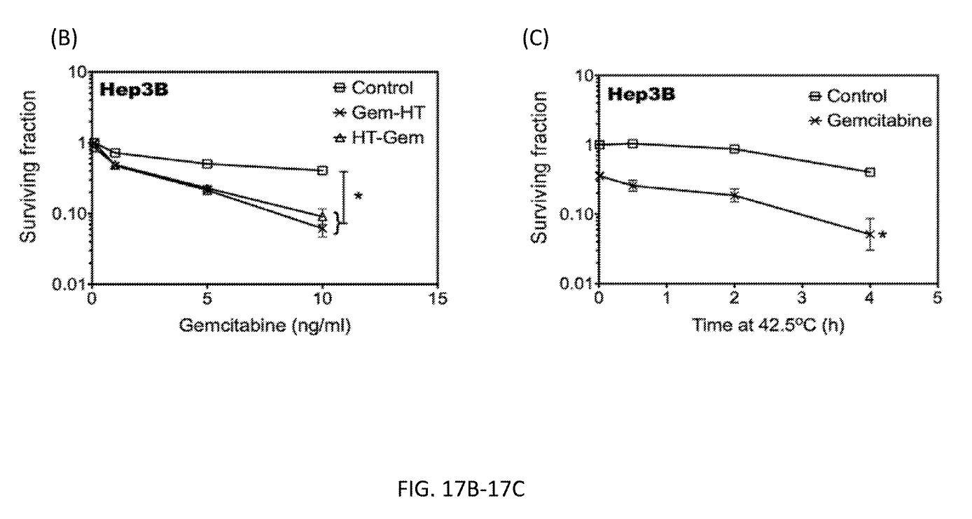

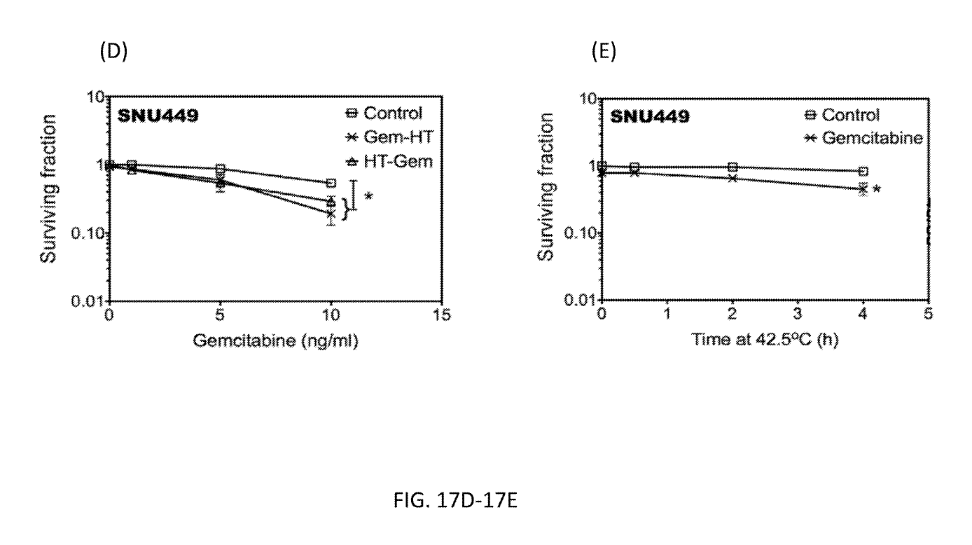

To further investigate that hyperthermia would inhibit the repair of gemcitabine-stalled replication forks and thus delay progression through the cell cycle, Hep3B cells were exposed to gemcitabine for 24 hours (approximate doubling time of Hep3B cells) at a concentration of 1 μM. This concentration is comparable to the peak intracellular concentration achieved with clinically used fixed-dose rate regimens (Mane, et al., 2010). Because incorporation of gemcitabine only occurs during S-phase, exposing Hep3B cells for 24 hours ensures incorporation of gemcitabine in all cells. For the last two hours of incubation, cells were or were not exposed to hyperthermia at 42.5° C. Cell cycle progression was analyzed over time (FIGS. 16 and 17).

A 24-hour incubation with 1 μM gemcitabine completely halts cell cycle progression by activating a Gl/S checkpoint (FIG. 16). Once gemcitabine is removed, cells resume synchronized DNA synthesis in 24 hours, suggesting that gemcitabine-induced cell cycle arrest is reversible at clinically relevant concentrations. The delay caused by hyperthermia in progression through early and mid S-phase was negligible. Gemcitabine followed by hyperthermia causes a much slower progression through late-S and G2/M phases compared with treatment with gemcitabine alone. The effect of hyperthermia on repair of stalled replication forks is transient (lasting a few hours), as most cells progress to the G0/G1 phase eventually.

Cells that fail to resolve stalled replication forks should demonstrate persistent staining for γ-H2AX phosphorylation sites. Hep3B cells were treated with gemcitabine (100 nM) for 24 hours. For the last two hours, cells were treated with or without hyperthermia at 42.5° C. The medium was then replaced and cells were allowed to recover. When cells were treated with a combination of gemcitabine and hyperthermia, γ-H2AX positive cells persisted for a longer duration compared with treatment with hyperthermia or gemcitabine alone (FIG. 16). The resolution of γ-H2AX positivity coincided with the progression of cell cycle observed previously.

Clonogenic Survival and Viability

Clonogenic assays were performed on Hep3B cells and SNU449 cells after one of two combinations of hyperthermia and gemcitabine compared with hyperthermia or gemcitabine alone. FIG. 17 shows a dose-dependent enhancement of gemcitabine toxicity by hyperthermia in both SNU449 and Hep3B cells irrespective of the dose schedule used. Next, clonogenic viability was evaluated at a gemcitabine concentration of 5 ng/ml and varied the duration of hyperthermia (30 minutes to 4 hours). The data demonstrate a thermal dose-dependent enhancement in toxicity for gemcitabine and hyperthermia.

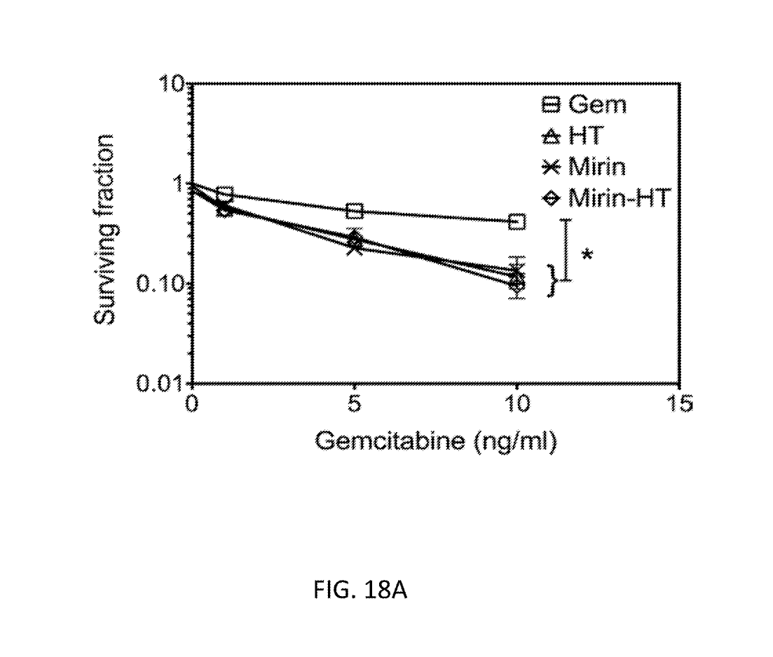

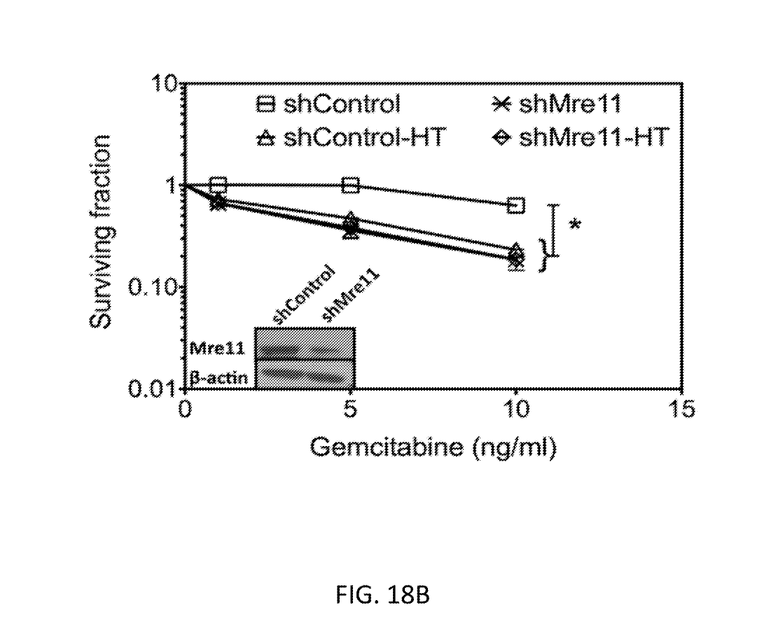

Because Mre11 is a thermolabile target of hyperthermia, it was considered whether inhibition of Mre11 exonuclease activity by a specific inhibitor, mirin, would result in similar enhancement of gemcitabine toxicity. Experiments were repeated with and without a sub-cytotoxic dose of mirin (25 μM) Inhibition of Mre11 exonuclease activity by mirin substantially enhanced gemcitabine-induced clonogenic cell death (FIG. 18). Addition of hyperthermia did not enhance this toxicity, suggesting that thermal enhancement of gemcitabine toxicity is mediated through an Mre11-dependent pathway.

To rule out the possibility of off-target effects of mirin, a partial Mre11 knockdown (shMre11) Hep3B cell line was developed. Western blot densitometry confirmed depletion of Mre11 by 72±11% (Mean, SD) in the shMre11 cell line compared with the shControl cell line. The shMre11 cell line was more sensitive to gemcitabine in comparison with the shControl cell line. Thermal enhancement of gemcitabine toxicity was noted for shControl but not shMre11 cells.

Animal Model Studies

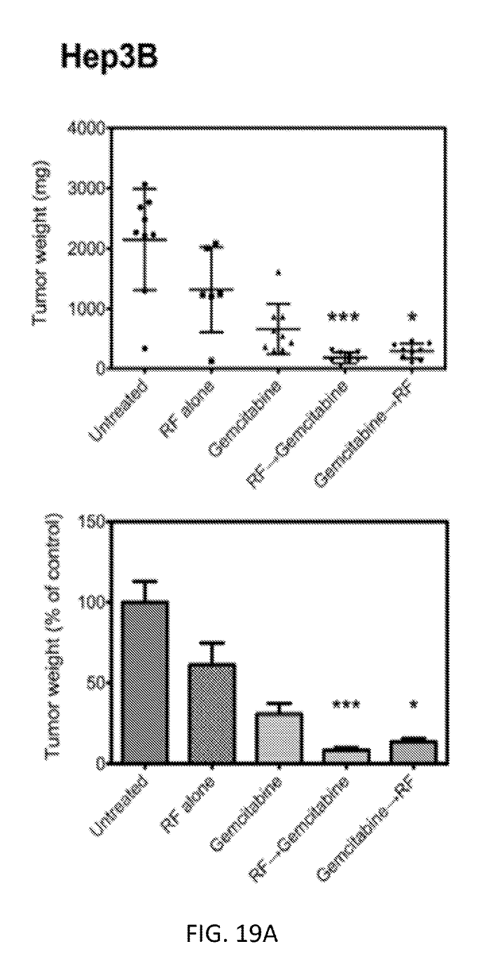

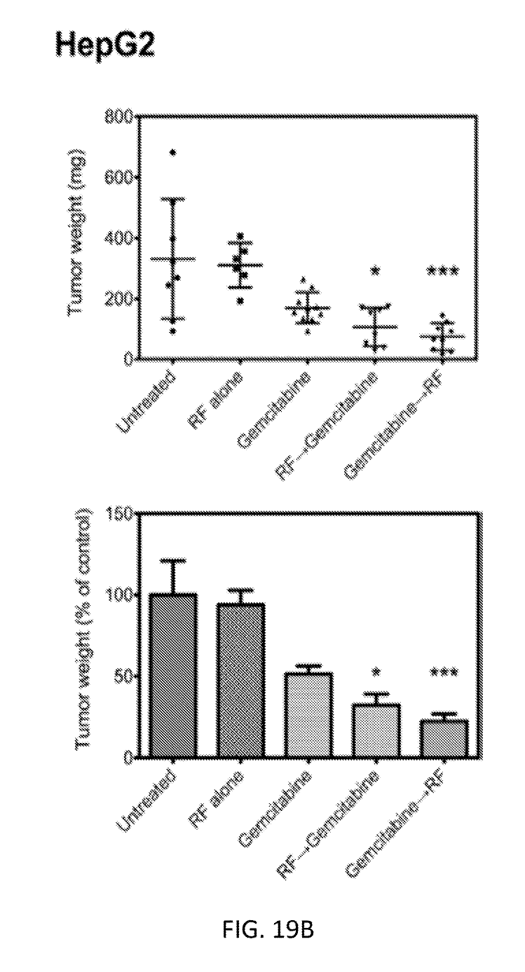

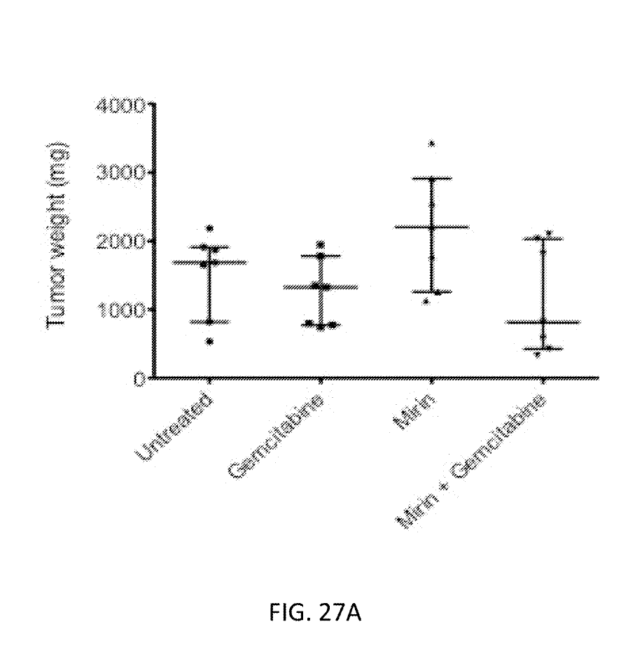

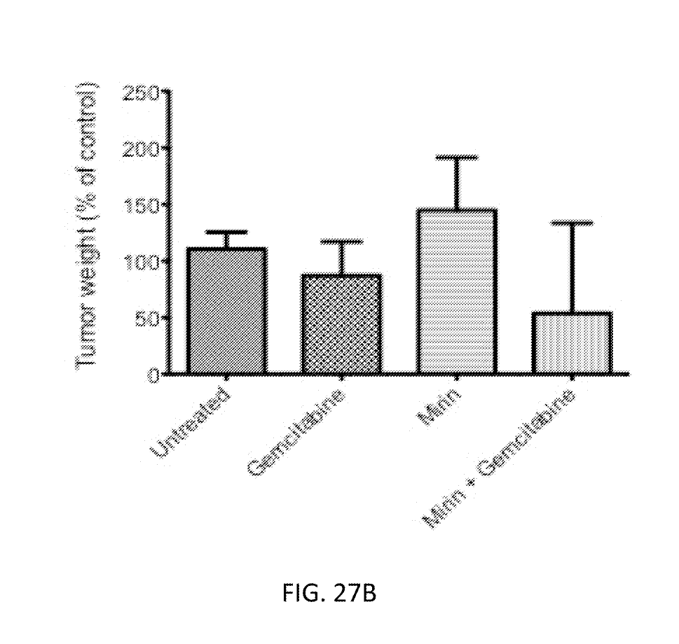

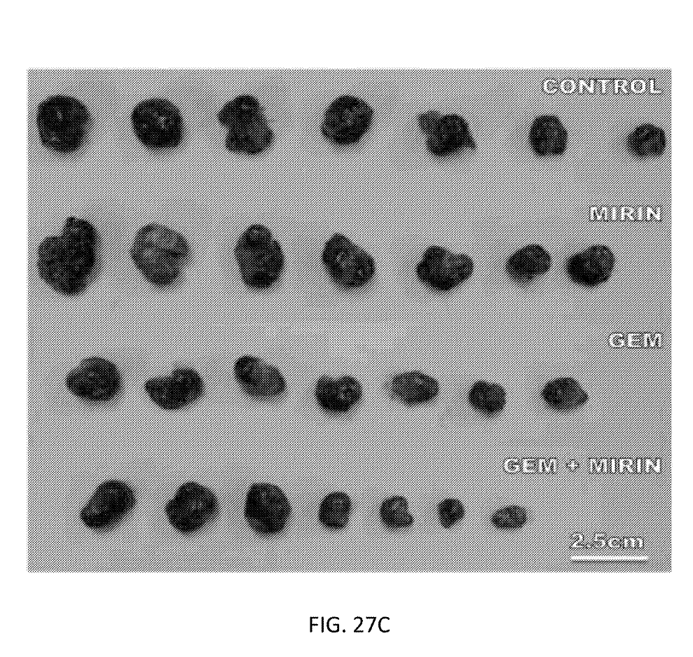

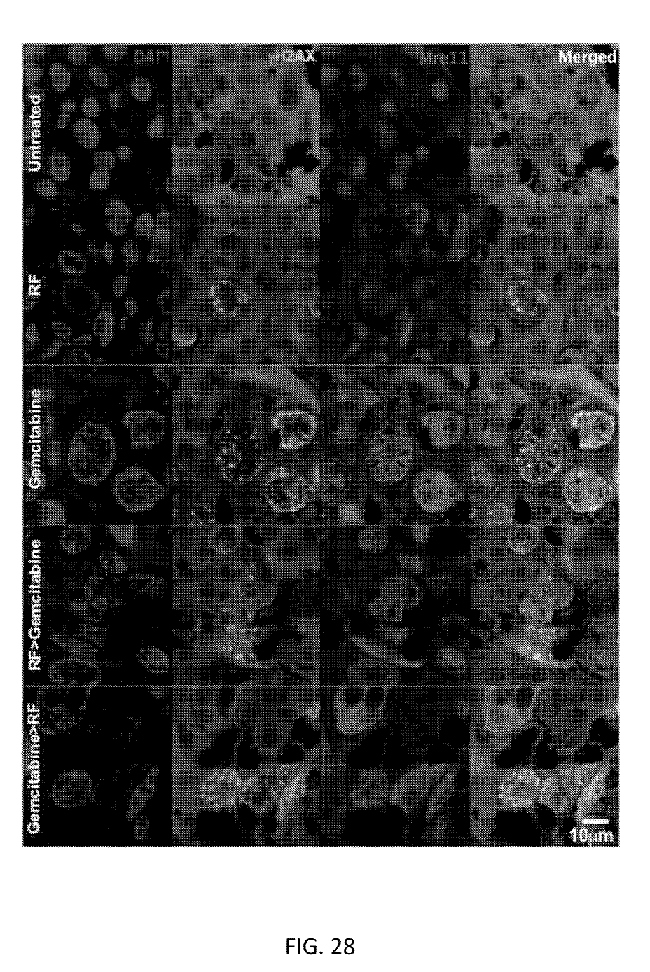

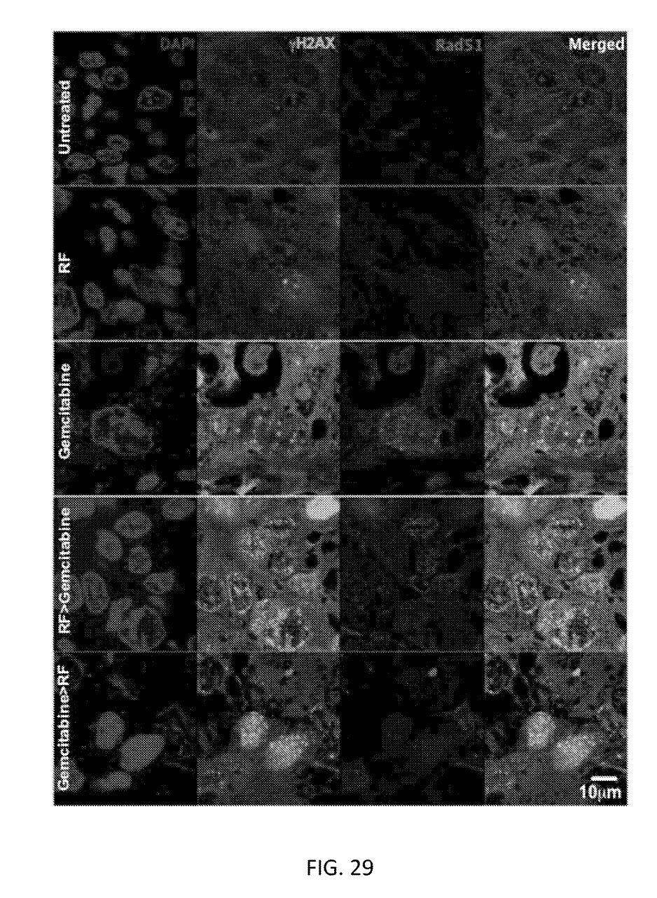

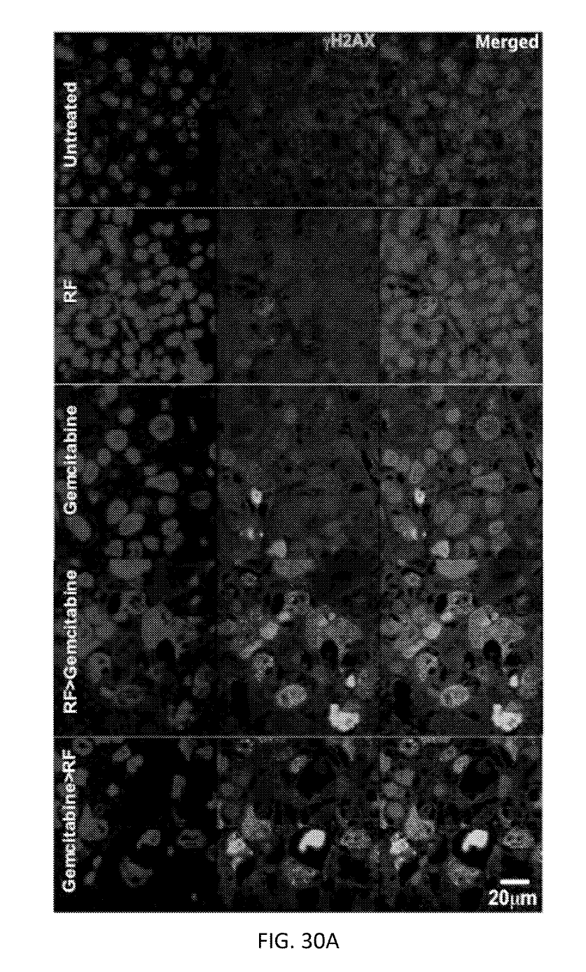

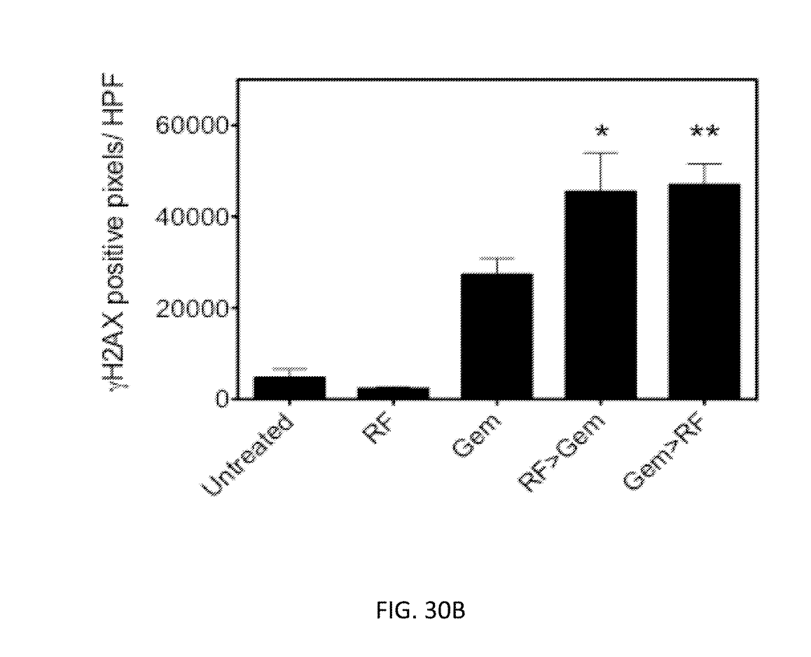

Development of mouse models and details of RF exposure and thermography are discussed in FIGS. 25 and 26. Mice bearing Hep3B orthotopic tumors were randomized to one of five groups: untreated, RF exposure alone, gemcitabine alone, gemcitabine followed 24 hours later by RF exposure, or RF exposure immediately followed by gemcitabine. The treatments were administered twice a week for three weeks for a total of six treatments. The gemcitabine dose administered was 70 mg/kg/dose or 150 mg/kg/week. This is approximately half the dose used in humans (1000 mg/m2/week dose in a 1.7 m human=˜300 mg/kg/week dose in a mouse). Twenty-four hours after the last treatment, mice were killed and tumors were harvested, weighed, and fixed in formalin for later analysis (FIG. 19A). Tumors in all treatment groups were statistically significantly smaller and had a lower tumor mass than untreated controls (P<0.05). Combination therapy was more effective than gemcitabine alone based on tumor mass, irrespective of the sequence of combination therapy (Mean tumor mass (±SD) for gemcitabine vs. RF then gemcitabine, 661±419 mg vs. 180±91 mg, P=0.0063; gemcitabine vs. gemcitabine then RF, 661±419 mg vs. 291±126 mg, P=0.022). This experiment was repeated in the slow-growing HepG2 xenograft model with weekly treatments for three weeks with similar results (FIG. 19B) (Mean tumor mass [±SD] for gemcitabine vs. RF then gemcitabine, 170±51 mg vs 107±64 mg, P=0.032; gemcitabine vs gemcitabine then RF, 170±51 mg vs 74±45 mg, P=0.0005). Preliminary in vivo experiments to assess the thermomimetic effect of mirin demonstrated that the mice treated with mirin and gemcitabine had the lowest median tumor mass compared with other groups (FIG. 27). Consistent with in vitro data, there was decreased localization of Mre11 and Rad51 to sites of stalled replication forks following RF-induced hyperthermia. There was a relatively higher proportion of γ-H2AX-positive cells in tumors treated with combination therapy in comparison with other groups (Mean γ-H2AX positive pixels/HPF [±SD] for gemcitabine vs. RF then gemcitabine, 27258±8022 vs 45430±18980, P=0.042; gemcitabine vs gemcitabine then RF, 27258±8022 vs 46968±10284 P=0.0097, FIGS. 28-30).

### Significance of Certain Embodiments

Several pathways have been implicated in chemoresistance of solid tumors to gemcitabine. Most involve reduced conversion of the prodrug (gemcitabine) to active drug (gemcitabine triphosphate), leading to decreased incorporation into DNA. The data demonstrate that a clinically achievable intracellular concentration (1 μM) was sufficient to arrest the cell cycle in hepatocellular carcinoma cells, suggesting adequate incorporation of gemcitabine into the DNA. Pathways to repair gemcitabine-stalled replication forks exist and may contribute to drug resistance.

Proteins of the HRR-pathway believed to be important in the restart of hydroxyurea-stalled replication forks were evaluated. RPA, Mre11 and Rad51 readily accumulate at sites of stalled replication forks. These findings are consistent with prior reports (Bryant, et al., 2009; Seno, et al., 2004; Ying, et al., 2012). When evaluating HRR protein levels after hyperthermia, partial degradation of Mre11 was consistently observed in all cell lines. Thermolability of Mre11 has been recently reported in a study where only 10 minutes at 42.5° C. reduced Mre11 exonuclease function to 10% of untreated control (Mane, et al., 2010). In addition, Mre11 and, as a result, downstream Rad51 failed to localize to gemcitabine-stalled replication forks in cells pretreated with mild hyperthermia. There was a prolonged passage of cancer cells treated with gemcitabine and hyperthermia through late S and G2 phase, which is characteristic for cells deficient in post-replication recombination repair (Su, et al., 2008). When evaluating synergy of gemcitabine and hyperthermia, Vertees et al. noted a similar enhancement of cells arrested in G2/M phase with combination therapy (Vertrees, et al., 2005). As a consequence, inhibition of the Mre11-dependent HRR pathway after exposure to gemcitabine by hyperthermia, mirin, or Mre11 knockdown is responsible for decreased clonogenic survival and cell death. A recent study demonstrated that nonhomologous end joining (NHEJ) represents a salvage pathway for cancer cells in which HRR of double-strand breaks is inhibited by hyperthermia (Bergs, et al., 2013). While this is a possibility, the role of NHEJ as an alternative fork restart pathway remains inconclusive (Allen, et al., 2011). In addition, the effect of hyperthermia on Mre-11 levels was not evaluated in that study. In some embodiments, inhibition of Mre-11 by hyperthermia also inhibits the salvage NHEJ pathway

It is important to discuss thermal degradation of BRCA2 and its effects on HRR of stalled replication forks because BRCA2 plays two essential roles at the stalled replication forks. BRCA2 prevents excessive nucleolytic degradation of stalled forks by Mre11, an effect associated with genomic instability (Zhuang, et al., 2009). Therefore, degradation of BRCA2 is expected to increase excision of gemcitabine by Mre11 and contribute to chemoresistance. Conversely, BRCA2 participates in Rad51 loading in the HRR pathway (Jensen, et al., 2010). In this case, degradation of BRCA2 is expected to inhibit the repair of gemcitabine-stalled replication forks and have the opposite effect. Because of this paradoxical effect at stalled replication forks, it is unlikely that BRCA2 is responsible for thermal enhancement of gemcitabine toxicity as observed here. In specific embodiments, synergistic interaction between hyperthermia and gemcitabine will ultimately depend on the relative effect of heat on BRCA2 compared with that on Mre11. For instance, thermal degradation of BRCA2 without Mre11 inhibition may not only contribute to gemcitabine resistance but also genomic instability as detailed in a report by Schlacher et al. (Schlacher, et al., 2011). Conversely, tumors already deficient in BRCA2 may be more susceptible to thermal sensitization of gemcitabine therapy. This speculation is supported by findings of Ying et al., who showed BRCA2 deficient cells to be more susceptible to Mre11 inhibition (Ying, et al., 2012).

Thus, the HRR pathway is important in the repair of gemcitabine-stalled replication forks. Inhibition of the HRR pathway protein Mre11 enhances the toxicity of gemcitabine in cancer cells in vitro and in vivo. Replication inhibitors similar to gemcitabine that result in stalling of the replication forks and activate HRR pathway are exploited in a similar manner, in particular embodiments. Examples include Cytosine arabinoside (AraC), Hydroxyurea (HU), Cepacitabine and other nucleoside inhibitors of replication. Thermal enhancement of the anti-tumor effect of gemcitabine is mediated through inhibition of Mre11-dependent HRR pathways by denaturation and degradation of Mre11. Inhibiting other HRR proteins, such as BRCA2, Rad51, PARP1, NBS, Rad50, could also potentially achieve thermal enhancement of anti-tumor effect of replication inhibitors. Noninvasive RF field-induced hyperthermia in combination with gemcitabine is superior to either modality alone in orthotopic mouse models of hepatocellular carcinoma.

### REFERENCES

All patents and publications cited herein are hereby incorporated by reference in their entirety herein. Citations for the references cited herein are provided in the following list.
Corr, et al., J Vis Exp. 2013, (78), e50480.
Corr, et al., The Journal of Physical Chemistry C 2012, 116, (45), 24380-24389.
Diop-Frimpong, et al., PNAS USA. 2011, 108, (7), 2909-2914.
Dongxiao, et al., Biomedical Engineering, IEEE Transactions on 2012, 59, (12), 3468-3474.
Franken, et al., Nat. Protoc. 2006, 1,2315-2319.
Gannon, et al., Cancer 2007, 110, (12), 2654-2665.
Gannon, et al., Journal of Nanobiotechnology 2008, 6, (1), 2.
Glazer, et al. Cancer 2010, 116, (13), 3285-3293.
Glazer, et al., Surgery 2010, 148, (2), 319-324.
Glazer, et al., Clin Cancer Res 2010, 16, 5712-5721.
Goldberg, et al., American journal of roentgenology 2002, 179, (1), 93-101.
Goldberg, et al., Academic radiology 1996, 3, (3), 212-8.
Hanson & Patch, Journal of Applied Physics 2009, 106, (5), 054309-054309-10.
Hanson, et al., Journal of Applied Physics 2011, 109, (12), -.
Kim, et al., Science 2013, 340, (6131), 441-442.
Koshkina, et al., Cancer 2014, 120, (4), 480-491.
Kruse, et al., Biomedical Engineering, IEEE Transactions on 2011, 58, (7), 2002-2012.
Kruskal, et al., JVIR 2001, 12, (10), 1193-201.
Li, et al., Journal of Colloid and Interface Science 2011, 358, (1), 47-53.
Raoof, et al., International Journal of Hyperthermia 2012, 28, (3), 202-209.
Raoof, et al., Nanomedicine: Nanotechnology, Biology and Medicine 2012, 8, (7), 1096-1105.
Raoof, et al., Biomaterials 2013, 34, (7), 1862-1869.
Raoof, et al., Nanomedicine: Nanotechnology, Biology, and Medicine 2014.
Raoof, et al., PLoS ONE 2013, 8, (7), e68506.
Ren, et al., Science Translational Medicine 2012, 4, (147), 147ra112.
Stephan, et al., Clin Cancer Res 2004, 10, (20), 6993-7000.
Yokoi, et al., Cancer Letters 2014, 345, (1), 48-55.
Vauthey, et al., Am J Surg. 1995; 169(1):28-34; discussion 34-25.
Soini, et al., J Clin Pathol. 1996; 49(6):470-473.
Huang, et al., J Natl Cancer Inst. 1992; 84(4):262-264.
Caruso, et al., Anticancer Res. 1999; 19(5B):3853-3856.
Llovet, et al., N Engl J Med. 2008; 359(4):378-390.
Huang, et al., Cancer Res. 1991; 51(22):6110-6117.
Gandhi, et al., Cancer Res. 1996; 56(19):4453-4459.
Helleday, Mutat Res. 2003; 532(1-2):103-115.
Bryant, et al., Embo J. 2009; 28(17):2601-2615.
Saintigny, et al., Embo J. 2001; 20(14):3861-3870.
Ewald, et al., Cancer Res. 2008; 68(19):7947-7955.
Zaalishvili, et al., Biochemistry (Mosc). 2012; 77(1):105-110.
Martin, et al., EMBO J. 2009; 28(22):3534-3548.
Zhu, et al., Radiat Res. 2001; 156(1):95-102.
Seno, et al., J Cell Physiol. 2004; 199(2):157-170.
Gerashchenko, et al., Cytometry A. 2010; 77(10):940-952.
Dynlacht, et al., Radiat Res. 2011; 176(3):323-332.
Krawczyk, et al., Proc Natl Acad Sci USA. 2011; 108(24):9851-9856.
Franken, et al., Nat Protoc. 2006; 1(5):2315-2319.
Glazer, et al., Clin Cancer Res. 2010; 16(23):5712-5721.
Warters, et al., Radiat Res. 1983; 93(1):71-84.
Mane, et al., Tumori. 2010; 96(3):405-410.
Ying, et al., Cancer Res. 2012; 72(11):2814.
Su, et al., Nat Struct Mol Biol. 2008; 15(10):1049-1058.
Vertrees, et al., Cancer Biol Ther. 2005; 4(10):1144-1153.
Bergs, et al., DNA Repair (Amst). 2013; 12(1):38-45.
Allen, et al., J Mol Cell Biol. 2011; 3(1):4-12.
Zhuang, et al., J Biol Chem. 2009; 284(44):30565-73.
Schlacher, et al., Cell 2011; 145(6):993
Jensen, et al., Nature. 2010; 467:678-683.

1. A method of overcoming or preventing resistance to one or more cancer drugs in an individual, comprising the step of providing to the individual an effective amount of radiofrequency therapy under sufficient conditions to the individual before, during, and/or after delivery of an effective amount of the one or more cancer drugs to the individual.

2. The method of claim 1, wherein the amount of the cancer drug delivered to the individual is less than the amount given to the individual in the absence of providing the radiofrequency therapy to the individual.

3. The method of claim 1, wherein the cancer drug is an alkylating agent, antimetabolite, anthracycline, topoisomerase inhibitor, mitotic inhibitor, proteasome inhibitor, or a combination thereof.

4. The method of claim 1, wherein the radiofrequency therapy and cancer drug are provided to the individual once a day.

5. The method of claim 1, wherein the radiofrequency therapy and cancer drug are provided to the individual more than once a day.

6. The method of claim 1, wherein the radiofrequency therapy and cancer drug are provided to the individual once a week.

7. The method of claim 1, wherein the radiofrequency therapy and cancer drug are provided to the individual more than once a week.

8. The method of claim 1, wherein the radiofrequency therapy and cancer drug are provided to the individual over the course of weeks or months.

9. The method of claim 1, wherein the radiofrequency therapy and cancer drug are provided to the individual over the course of 1-4, 1-3, 1-2, 2-4, 2-3, or 3-4 weeks.

10. The method of claim 1, wherein the radiofrequency therapy and cancer drug are provided to the individual over the course of 1-12, 1-11, 1-10, 1-9, 1-8, 1-7, 1-6, 1-5, 1-4, 1-3, 1-2, 2-12, 2-11, 2-10, 2-9, 2-8, 2-7, 2-6, 2-5, 2-4, 2-3, 3-12, 3-11, 3-10, 3-9, 3-8, 3-7, 3-6, 3-5, 3-4, 4-12, 4-11, 4-10, 4-9, 4-8, 4-7, 4-6, 4-5, 5-12, 5-11, 5-10, 5-9, 5-8, 5-7, 5-6, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 months.

11. The method of claim 1, wherein the radiofrequency therapy comprises a power between 200-1500 watts.

12. The method of claim 1, wherein the radiofrequency therapy is generated by a portable system.

13. The method of claim 1, wherein the duration of exposure of the radiofrequency therapy for the individual is between 5-10, 5-9, 5-8, 5-7, 5-6, 6-10, 6-10, 6-8, 6-7, 7-10, 7-9, 7-8, 8-10, 8-9, or 9-10 minutes.

14. The method of claim 1, wherein the temperature that is generated at a desired location to which the radiofrequency is directed is between 37° C. and 45° C.

15. The method of claim 1, wherein the individual is provided an additional cancer therapy.

16. The method of claim 15, wherein the additional cancer therapy comprises radiation, surgery, and/or a cancer composition that is not the cancer drug,.

17. The method of claim 1, wherein the cancer drug is gemcitabine, abraxane, cetuximab, nitrogen mustards, such as mechlorethamine (nitrogen mustard), chlorambucil, cyclophosphamide (Cytoxan®), ifosfamide; melphalan; nitrosoureas, such as streptozocin, carmustine (BCNU), and lomustine; alkyl sulfonates, such as busulfan; triazines, such as dacarbazine (DTIC) and temozolomide (Temodar®); ethylenimines, such as thiotepa and altretamine (hexamethylmelamine); 5-fluorouracil (5-FU); 6-mercaptopurine (6-MP); Capecitabine (Xeloda®); Cladribine; Clofarabine; Cytarabine (Ara-C®); Floxuridine; Fludarabine; Gemcitabine (Gemzar®); Hydroxyurea; Methotrexate; Pemetrexed (Alimta®); Pentostatin; Thioguanine; Daunorubicin; Doxorubicin (Adriamycin®); Epirubicin; Idarubicin; topotecan; irinotecan; or a combination thereof.

18. The method of claim 1, wherein the radiofrequency therapy is from a system that has in vivo imaging.

19. The method of claim 1, wherein the in vivo imaging is real-time in vivo imaging.

20. The method of claim 1, further comprising the step of imaging of a labeled compound used for therapy and/or imaging one or more biological changes that occur as a result of use of the therapy.

21. The method of claim 18, wherein the imaging comprises confocal microscopy, intravital microscopy, and/or multiphoton microscopy.

22. The method of claim 20, wherein the one or more biological changes comprises quantitation of vascular permeability, tissue integrity, nanoparticle accumulation, tissue penetration, tissue necrosis, tissue apoptosis, tissue DNA replication phase, blood flow dynamics, cellular migration events, one or more changes in tumor environment, one or more changes in tumor microenvironment, or a combination thereof.

23. The method of claim 1, further comprising the step of determining that the individual has cancer.

24. The method of claim 1, wherein the method occurs in the absence of providing one or more radiofrequency absorption enhancers to the individual.

25. The method of claim 1, wherein the radiofrequency occurs at
a) high-frequency radiowaves;
b) a medically-approved research frequency;
c) 13.56 MHz radiowaves amplitude-modulated at lower frequencies across the range of 20 Hz-13.55 MHz; or
d) pulsed radiowaves of pulse duration 1 ns-1 s.

26. The method of claim 25, wherein the high-frequency radiowave is about 13.56 MHz.

27. The method of claim 25, wherein the medically-approved research frequency is in the range of 20 HZ-1 GHz.

28. A method of treating cancer in an individual, comprising the step of providing to the individual a therapy that comprises, consists of, or consists essentially of an effective amount of radiofrequency therapy under sufficient conditions to the individual before, during, and/or after delivery of an effective amount of the cancer drug to the individual.

29. A method of increasing perfusion in tissues and/or cells of an individual of one or more cancer drugs that are delivered to the individual systemically or non-systemically, comprising the step of providing to the individual an effective amount of radiofrequency therapy to the individual before, during, and/or after delivery of an effective amount of the one or more cancer drugs to the individual.

30. The method of claim 29, wherein the cancer drug and radiofrequency therapy are provided to the individual at the same time.

31. The method of claim 29, wherein the cancer drug and radiofrequency therapy are provided to the individual at different times.

32. The method of claim 31, wherein the cancer drug is provided to the individual before the radiofrequency therapy.

33. The method of claim 31, wherein the cancer drug is provided to the individual after the radiofrequency therapy.

34. A method of increasing intra-tumoral blood vessel permeability for uptake of one or more cancer drugs in an individual, comprising the step of providing to the individual an effective amount of radiofrequency therapy to the individual before, during, and/or after delivery of an effective amount of the one or more cancer drugs to the individual.

35. The method of claim 34, wherein the increase is measured by improvement of at least one symptom of the cancer, by reduction in the size of a tumor, by reduction in metastasis of the cancer, by reduction in the tumor load, by reduction in the amount of cancer drug(s) that is effective for cancer treatment, and a combination thereof.

36. A method of enhancing tumor blood flow and delivery of one or more compositions to a tumor in an individual, comprising the step of providing to the individual an effective amount of radiofrequency therapy to the individual before, during, and/or after delivery of an effective amount of the one or more compositions to the individual.

37. The method of claim 36, wherein the composition is a drug, biologic agent, and/or nanomaterial.

38. A method of monitoring a radiofrequency therapy with thermography in an individual, comprising the steps of:
providing to the individual an effective amount of radiofrequency therapy to the individual before, during, and/or after delivery of an effective amount of the one or more cancer drugs to the individual; and
monitoring with thermography at least part of the individual.

39. The method of claim 38, wherein the thermography is magnetic resonance imaging thermography.

40. A system, comprising
a) a radiofrequency device; and
b) one or more cancer therapy compositions.

41. The system of claim 40, further comprising:
c) nanoparticles; and/or
d) a thermography device.

42. The system of claim 40, wherein the system lacks one or more radiofrequency absorption enhancers.
